(19) United States
(12) Patent Application Publication
(10) Pub. No.: US 2005/0251872 A1
(43) Pub. Date: Nov. 10, 2005

(54) Lentiviral vectors, related reagents, and methods of use thereof

(76) Inventors: James E. Bear, Durham, NC (US); Christopher P. Dillon, Cambridge, MA (US); Douglas A. Rubinson, Cambridge, MA (US); Luk Van Parijs, Falmouth, MA (US)

Correspondence Address: CHOATE, HALL & STEWART LLP, TWO INTERNATIONAL PLACE, BOSTON, MA, 02110, US

(21) Appl. No.: 10/655,872

(22) Filed: Sep. 5, 2003

Related U.S. Application Data
(60) Provisional application No. 60/408,558, filed on Sep. 6, 2002.
(60) Provisional application No. 60/414,195, filed on Sep. 27, 2002.
(60) Provisional application No. 60/428,039, filed on Nov. 21, 2002.

(51) Int. Cl. A01K 67/00; C12N 7/00; C12N 15/867

(52) U.S. Cl. 800/8; 435/456; 435/235.1

(57) ABSTRACT

The present invention provides new lentiviral vectors, including lentiviral transfer plasmids and infectious lentiviral particles. Lentiviral vectors of the invention were designed to offer a number of desirable features including reduced size, convenient cloning sites (including multiple cloning sites and sites for particularly useful restriction enzymes), loxP sites, self-inactivating LTRs, etc. Certain of the vectors are optimized for expression of reporter genes and/or for expression of siRNAs or shRNAs within eukaryotic cells. The invention also provides three and four plasmid lentiviral expression systems. In addition, the invention provides a variety of methods for using the vectors including gene silencing in cells and transgenic animals, and methods of treating disease.

### CROSS-REFERENCE TO RELATED APPLICATIONS

This application claims priority to U.S. Provisional Patent Applications Ser. No. 60/408,558, filed Sep. 6, 2002, Ser. No. 60/414,195, filed Sep. 27, 2002, and Ser. No. 60/428,039, filed Nov. 21, 2002. The contents of each of these applications is incorporated herein by reference.

### BACKGROUND OF THE INVENTION

Viral vectors are efficient gene delivery tools in eukaryotic cells. Useful viral vectors have been created from different virus families, including retroviruses. Retroviruses have proven to be versatile and effective gene transfer vectors for a variety of applications since they are easy to manipulate, typically do not induce a strong anti-viral immune response, and are able to integrate into the genome of a host cell, leading to stable gene expression. If provided with an appropriate envelope, retroviruses can infect almost any type of cell. Due to these advantages a large number of retroviral vectors have been developed for in vitro gene transfer. In addition, use of retroviruses for purposes such as the creation of transgenic or knockout animals, or for gene therapy, has been explored.

However, vectors based on simple retroviruses (e.g., oncoretroviruses) have a number of disadvantages that limit their efficacy for such in vivo applications. For example, vectors based on simple retroviruses are generally unable to integrate into the genome of nondividing (postmitotic) cells. Furthermore, transgenes expressed from simple retroviruses are subject to silencing during development (22). To overcome these drawbacks, attention has recently focused on lentiviruses, a group of complex retroviruses that includes the human immunodeficiency virus (HIV). In addition to the major retroviral genes gag, pol, and env, lentiviruses typically include additional genes that play regulatory or structural roles. Unlike simple retroviruses, lentiviruses are able to integrate into the genome of non-dividing cells. Accordingly a variety of lentiviral vectors have been developed. However, existing lentiviral vectors remain less than optimal from a number of perspectives. For example, existing lentiviral vectors are typically large in size, poorly characterized, and lack various features that facilitate cloning and uses of the vectors. Thus there remains a need in the art for improved lentiviral vectors. The present invention addresses this need.

Rapid progress in technologies for sequencing genes and characterizing their expression profiles has resulted in a growing list of coding regions within mammalian genomes that are predicted to contribute to normal tissue function and to the development of disease. Traditionally, establishing gene function has been accomplished by gene targeting in mouse embryonic stem cells. While this technology has been responsible for many key breakthroughs in our understanding of the normal function as well as diseases of organs and tissues, it remains time-consuming and expensive to perform. Furthermore, current gene targeting approaches cannot be used to alter gene function in human tissues for the purposes of scientific investigation or gene therapy. For these reasons, alternative approaches to inhibit gene activity in primary cells and tissues have been explored.

Among the most promising of these new approaches is RNA interference (RNAi), which has recently emerged as a rapid and efficient means to silence gene function in eukaryotic (including mammalian) cells. As initiallyy described in the nematode C. elegans, RNAi involves introduction of double-stranded RNA (dsRNA) into a cell thereby inhibiting gene expression in a sequence dependent fashion. More recently it has been shown that shorter dsRNA species known as short interfering RNAs (siRNA) can silence mammalian gene expression in a specific manner, suggesting that RNAi can be used to study and manipulate gene function in higher organisms as well. However, the use of RNAi in mammalian cells and organisms is currently restricted by the limited delivery methods available. Accordingly, there is a need in the art for improved reagents and methods that would facilitate the use of RNAi in mammalian cells and organisms. The present invention addresses this need, among others.

### SUMMARY OF THE INVENTION

The present invention provides novel lentiviral vectors that offer a number of features and advantages. In one aspect, the invention provides a lentiviral vector comprising the following elements: a nucleic acid whose sequence includes (i) a functional packaging signal; (ii) a multiple cloning site (MCS); and (iii) at least one additional element selected from the group consisting of: a second MCS, a second MCS into which a heterologous nucleic acid is inserted, a human immunodeficiency (HIV) FLAP element, an expression-enhancing posttranscriptional regulatory element, a target site for a site-specific recombinase, and a self-inactivating (SIN) long terminal repeat (LTR). The lentiviral vector may be a lentiviral transfer plasmid or an infectious lentiviral particle. In various embodiments of the invention the expression-enhancing posttranscriptional regulatory element is a woodchuck hepatitis virus regulatory element (WRE), and/or the target site is a loxP site. The invention further provides collections of lentiviral plasmids possessing the features described above.

In other aspects, the invention provides cells, including mammalian cells, and transgenic animals that contain any of the inventive lentiviral vectors or proviruses derived therefrom. The invention further provides methods for making transgenic animals the cells of which comprise an inventive lentiviral vector or a provirus derived therefrom.

The invention further provides a variety of lentiviral expression systems comprising inventive lentiviral transfer plasmids. For example, the invention provides a three-plasmid lentiviral expression system comprising: (a) a first plasmid whose sequence comprises a nucleic acid sequence of at least part of a lentiviral genome, wherein the plasmid (i) contains at least one defect in at least one gene encoding a lentiviral structural protein, and (ii) lacks a functional packaging signal; (b) a second plasmid whose sequence comprises a nucleic acid sequence of a virus, wherein the plasmid (i) expresses a viral envelope protein, and (ii) lacks a functional packaging signal; and (c) a third plasmid whose nucleic acid sequence includes (i) a functional packaging signal; (ii) a multiple cloning site (MCS); and (iii) at least one additional element selected from the group consisting of: a second MCS, a second MCS into which a heterologous nucleic acid is inserted, an HIV FLAP element, an expression-enhancing posttranscriptional regulatory element, a target site for a site-specific recombinase, and a self-inactivating (SIN) LTR.

The invention further provides a four plasmid lentiviral expression system, in which three of the plasmids are as described immediately above and the fourth plasmid encodes the Rev protein.

The invention provides methods of creating infectious lentiviral particles and of creating producer cell lines that produce infectious lentiviral particles. The lentiviral particles may, but need not be, derived from the lentiviral transfer plasmids as described herein.

The invention further provides a method for introducing and expressing a heterologous nucleic acid in a target cell comprising introducing a lentiviral vector of the invention into the target cell and expressing the heterologous nucleic acid therein. In various embodiments of the invention the heterologous nucleic acid is operably linked to a constitutive, an inducible, or a cell type or tissue specific promoter, allowing conditional expression of the nucleic acid.

In another aspect, the invention provides a method for achieving controlled expression of a heterologous nucleic acid in a cell comprising steps of: (i) inserting the heterologous nucleic acid into a lentiviral vector between sites for a recombinase, thereby producing a modified lentiviral vector; (ii) introducing the modified lentiviral vector or a portion thereof including at least the sites for the recombinase and the region between the sites into the cell and; (iii) subsequently inducing expression of the recombinase within the cell, thereby preventing expression of the heterologous nucleic acid within the cell.

The invention also provides a method for expressing a transcript in a mammal in a cell type or tissue-specific manner comprising: (i) delivering a lentiviral vector to cells of the mammal, wherein the lentiviral vector comprises a heterologous nucleic acid, and wherein the heterologous nucleic acid is located between sites for a site-specific recombinase; and (ii) inducing expression of the site-specific recombinase in a subset of the cells of the mammal, thereby preventing synthesis of the transcript within those cells.

In another aspect, the invention provides a lentiviral vector whose presence within a cell results in transcription of one or more ribonucleic acids (RNAs) that self-hybridize or hybridize to each other to form a short hairpin RNA (shRNA) or short interfering RNA (siRNA) that inhibits expression of at least one target transcript in the cell. In certain embodiments of the invention the lentiviral vector comprises a nucleic acid segment operably linked to a promoter, so that transcription from the promoter (i.e., transcription directed by the promoter) results in synthesis of an RNA comprising complementary regions that hybridize to form an shRNA targeted to the target transcript. (When an RNA comprises complementary regions that hybridize with each other, the RNA will be said to self-hybridize.) According to certain embodiments of the invention the shRNA comprises a base-paired region approximately 19 nucleotides long. According to certain embodiments of the invention the RNA may comprise more than 2 complementary regions, so that self-hybridization results in multiple base-paired regions, separated by loops or single-stranded regions. The base-paired regions may have identical or different sequences and thus may be targeted to the same or different regions of a single transcript or to different transcripts.

In certain embodiments of the invention the lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. According to certain embodiments of the invention the siRNA comprises a base-paired region approximately 19 nucleotides long. In certain embodiments of the invention the lentiviral vector comprises at least two promoters and at least two nucleic acid segments, wherein each promoter is operably linked to a nucleic acid segment, so that transcription from the promoters results in synthesis of two complementary RNAs that hybridize with each other to form an siRNA targeted to the target transcript. The nucleic acid segment(s) present within the lentiviral vectors may be part of a larger nucleic acid, e.g., a heterologous nucleic acid that is inserted into the vector as described herein.

The lentiviral vectors of the invention may be lentiviral transfer plasmids or infectious lentiviral particles (e.g., a lentivirus or pseudotyped lentivirus). As discussed further below, lentiviruses have an RNA genome. Therefore, where the lentiviral vector is a lentiviral particle, the viral genome must undergo reverse transcription and second strand synthesis to produce DNA capable of directing RNA transcription. In addition, where reference is made herein to elements such as cloning sites, promoters, regulatory elements, etc., it is to be understood that the sequences of these elements are present in RNA form in the lentiviral particles of the invention and are present in DNA form in the lentiviral transfer plasmids of the invention. Furthermore, where a template for synthesis of an RNA is “provided by” RNA present in a lentiviral particle, it is understood that the RNA must undergo reverse transcription and second strand synthesis to produce DNA that can serve as a template for synthesis of RNA (transcription).

The invention further provides pharmaceutical compositions comprising any of the inventive lentiviral vectors and a pharmaceutically acceptable carrier.

The invention further provides a three plasmid lentiviral expression system comprising (i) a lentiviral transfer plasmid, wherein the lentiviral transfer plasmid directs transcription of at least one ribonucleic acid (RNA) that, when present within a cell, hybridizes to form an shRNA or siRNA that inhibits expression of at least one gene expressed in the cell. (ii) a packaging plasmid; and (iii) an Env-coding plasmid. In certain embodiments of the invention the lentiviral transfer plasmid comprises a nucleic acid segment operably linked to a promoter, so that transcription from the promoter results in synthesis of an RNA that hybridizes to form an shRNA targeted to a target transcript. In certain embodiments of the invention the lentiviral transfer plasmid comprises a nucleic acid segment flanked by two oppositely directed promoters, wherein the promoters are operably linked to the nucleic acid segment, so that transcription from the promoters results in synthesis of two complementary RNAs that hybridize with each other to form an siRNA targeted to a target transcript. In certain embodiments of the invention the lentiviral transfer plasmid comprises two promoters and two nucleic acid segments, wherein each promoter is operably linked to a nucleic acid segment, so that transcription from the promoters results in synthesis of two complementary RNAs that hybridize with each other to form an siRNA targeted to a target transcript. The lentiviral transfer plasmid may, but need not be, any of the inventive lentiviral transfer plasmids described herein.

The invention further provides a four plasmid lentiviral expression system comprising a three plasmid lentiviral expression system as described above and a fourth plasmid that encodes the Rev protein.

The invention additionally provides a method of inhibiting or reducing the expression of a target transcript in a cell comprising delivering a lentiviral vector to the cell, wherein presence of the lentiviral vector within the cell results in synthesis of one or more RNAs that self-hybridize or hybridize with each other to form an shRNA or siRNA that inhibits expression of the target transcript. Note that where presence of the lentiviral vectors, particles, or plasmids of the invention results in production of an shRNA, the shRNA may require further processing within the cell to form an inhibitory structure. shRNAs that are so processed are considered to inhibit expression of the target transcript.

The invention further provides a method for reversibly inhibiting or reducing expression of a target transcript in a cell comprising: (i) delivering a lentiviral vector to the cell, wherein presence of the lentiviral vector within the cell results in synthesis of one or more RNAs that self-hybridize or hybridize with each other to form an shRNA or siRNA that inhibits expression of the target transcript, wherein the lentiviral vector comprises a nucleic acid segment located between sites for a site-specific recombinase, which nucleic acid segment provides a template for transcription of the one or more RNAs; and (ii) inducing expression of the site-specific recombinase within the cell, thereby preventing synthesis of at least one of the RNAs. The vector can be a lentiviral transfer plasmid or lentiviral particle.

The invention also provides a method for reversibly inhibiting or reducing expression of a transcript in a mammal in a cell type or tissue-specific manner comprising: (i) delivering to the mammal a lentiviral vector whose presence within a cell results in synthesis of one or more RNAs that self-hybridize or hybridize with each other to form an shRNA or siRNA that inhibits expression of the target transcript, wherein the lentiviral vector comprises a nucleic acid segment located between sites for a site-specific recombinase, which nucleic acid segment provides a template for transcription of the RNA; and (ii) inducing expression of the site-specific recombinase in a subset of the cells of the mammal, thereby preventing synthesis of at least one of the RNAs within the subset of cells. In any of the above methods, the cell may be a mammalian cell, the site-specific recombinase may be Cre, and the sites may be loxP sites.

The invention includes a variety of therapeutic applications for the inventive lentiviral vectors. In particular, the lentiviral vectors are useful for gene therapy. The invention provides a method of treating or preventing infection by an infectious agent, the method comprising the step of administering to a subject prior to, simultaneously with, or after exposure of the subject to the infectious agent, a composition comprising an effective amount of a lentiviral vector, wherein the lentiviral vector directs transcription of at least one RNA that hybridizes to form an shRNA or siRNA that is targeted to a transcript produced during infection by the infectious agent, which transcript is characterized in that reduction in levels of the transcript delays, prevents, or inhibits one or more aspects of infection by or replication of the infectious agent.

In addition, the invention provides a method of treating or preventing a disease or clinical condition, the method comprising: (i) removing a population of cells from a subject at risk of or suffering from disease or clinical condition; (ii) engineering or manipulating the cells to contain an effective amount of an siRNA or shRNA targeted to a transcript by infecting or transfecting the cells with a lentiviral vector, wherein the transcript is characterized in that its degradation delays, prevents, or inhibits one or more aspects of the disease or clinical condition; (iii) and returning at least a portion of the cells to the subject. Suitable lentiviral vectors are described herein. Without intending to suggest any limitation, the therapeutic approaches may find particular use in diseases such as cancer, in which a mutation in a cellular gene is responsible for or contributes to the pathogenesis of the disease, and in which specific inhibition of the target transcript bearing the mutation may be achieved by expressing an siRNA or shRNA targeted to the target transcript within the cells, without interfering with expression of the normal allele. According to certain embodiments of the invention, rather than removing cells from the body of a subject, infecting or transfecting them in tissue culture and then returning them to the subject, inventive lentiviral vectors or lentiviruses are delivered directly to the subject.

This application refers to various patents, journal articles, and other publications, all of which are incorporated herein by reference. In addition, the following publications are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001.

### BRIEF DESCRIPTION OF THE DRAWING

FIG. 1 shows a map of pBFGW.

FIG. 2 shows a map of pLL3.0.

FIG. 3 shows a map of pLL3.1.

FIG. 4 shows a map of pLL3.2.

FIG. 5 shows a map of pLL3.3.

FIG. 6 shows a map of pLL3.4.

FIG. 7 shows a map of pLL3.5.

FIG. 8 shows a map of pLL3.6.

FIG. 9 shows a map of pLL3.7.

FIG. 10A shows schematic diagrams of the HIV provirus (upper panel) and relevant portions of representative packaging and Env-coding plasmids (middle and lower panels, respectively) for a three plasmid system.

FIG. 10B shows schematic diagrams of the HIV provirus (upper panel) and relevant portions of representative packaging, Rev-coding and Env-coding plasmids (second, third, and lower panels, respectively) for a four plasmid system.

FIG. 11 shows the siRNA structure found to be active in the Drosophila system.

FIG. 12 presents a schematic representation of the steps involved in RNA interference in Drosophila.

FIG. 13 shows a schematic diagram of a variety of exemplary shRNA structures useful in accordance with the present invention.

FIG. 14 presents a representation of an alternative inhibitory pathway, in which the DICER enzyme cleaves a substrate having a base mismatch in the stem to generate an inhibitory product that binds to the 3′ UTR of a target transcript and inhibits translation.

FIG. 15 presents a schematic diagram of a nucleic acid that serves as a template for transcription of an RNA that hybridizes to form an shRNA and also shows the RNA before and after hybridization.

FIG. 16 presents a schematic diagram of one example of a construct that may be used to direct transcription of sense and antisense strands of an siRNA.

FIG. 17A presents a schematic representation of a portion of the lentivirus vector pLL3.7. Key: SIN-LTR: self-inactivating long terminal repeat; Ψ: HIV packaging signal; cPPT: central polypurine track; U6: U6 (RNA polymerase III) promoter; MCS: multiple cloning site; CMV: cytomegalovirus (RNA polymerase II) promoter; EGFP: enhanced green fluorescent protein; WRE: woodchuck hepatitis virus response element.

FIG. 17B presents the sequence of the CD8 stem loop used to generate pLL3.7 CD8 (See Examples). A sequence known to silence CD8 as an siRNA (11) was adapted with a loop sequence from Paddison et al. (39) to create the final sequence. The presumed transcription initiation site is indicated by a +1. Nucleotides which form the loop structure are indicated in green font (Loop). The pol III terminator stretch (a stretch of Us in the RNA) is indicated in red font.

FIG. 17C shows the predicted structure of the CD8 stem-loop RNA produced from pLL3.7 CD8.

FIG. 18A shows density plots demonstrating specific silencing of CD8 expression by pLL3.7 CD8. CD8⁺CD4⁺E10 cells were either mock infected (No Virus), infected with a pLL3.7 (Control Virus), or pLL3.7 CD8 (CD8 RNAi virus). Density plots indicate the expression levels of CD4 and CD8 48 hours post-infection.

FIG. 18B presents histograms showing staining for the T cell surface markers, CD3, TCRβ, and CD28. The histograms show that other surface markers are unaffected by silencing of CD8. E10 cells infected with pLL3.7 (green histograms) or pLL3.7 CD8 (pink histograms) were stained for CD3, TCRβ, and CD28. Solid histograms represent the level of these surface markers on uninfected cells.

FIG. 19A shows stable silencing of CD8 by pLL3.7 CD8. Sorted populations of infected E10 cells were maintained in long-term culture. E10 cells pLL3.7 CD8 (CD8 RNAi virus) were sorted four days after infection for GFP expression and low CD8 expression, while cells infected with control virus were sorted for GFP expression only. Each population was cultured for 1 month and analyzed for CD8 expression via flow cytometry at weekly intervals. The CD8 and GFP levels expressed by infected cells 4 days following infection and after one month of culture are shown.

FIG. 19B shows a Northern blot showing specific degradation of CD8 mRNA induced by pLL3.7 CD8. CD8 and CD4 mRNA levels in uninfected E10 cells, or E10 cells infected with either pLL3.7 (Control Virus) or pLL3.7 CD8 (CD8 RNAi Virus) and sorted on the basis of GFP and CD8 expression, were assayed. The bands representing CD8 and CD4 mRNA species are identified by lines (top panel).

FIG. 19C shows generation of processed shRNAs in cells infected with pLL3.7 CD8. The cells analysed for CD8 and CD4 mRNA levels described in the legend to FIG. 18B were also assayed for the presence of shRNAs by Northern blot. The location of 21, 22, and 23 nucleotide RNAs are identified by arrows.

FIG. 20A presents flow cytometric analysis showing specific silencing of genes in primary T cells by pLL3.7 CD8 and pLL3.7 CD25. CD8⁺ TCR transgenic T cells were activated for 3 days with cognate peptide and then infected with pLL3.7, pLL3.7 CD8, or pLL3.7 CD25. The efficiency of infection was determined by assaying GFP expression by flow cytometry. The expression of CD8 and CD25 on infected T cells was assayed by staining with specific antibodies that bind these surface markers.

FIG. 20B is a bar graph showing functional silencing of genes in primary T cells with pLL3.7 CD25. CD8⁺ TCR transgenic T cells were infected and activated as in A. and then cultured for 48 hours in the presence of increasing concentrations of IL-2. Proliferation was assessed by ³H-thymidine incorporation.

FIG. 21A shows flow cytometric analysis of expression of GFP from pLL3.7 CD8 infection in the AK7 ES cell line. AK7 ES cells were infected with pLL3.7 CD8 and sorted for GFP expression (green line) and compared with uninfected control (purple peak).

FIG. 21B shows fluorescent imaging of paws of ES cell-derived mice. The paws of control and pLL3.7 CD8 ES chimeric mice were imaged with standard epifluorescence for expression of EGFP.

FIG. 21C shows flow cytometric identification of ES cell-derived thymocytes in chimeric mice. Thymocytes from noninfected (purple peak) and pLL3.7 CD8 (green line) ES derived mice were harvested and analyzed for GFP expression.

FIG. 21D is a photograph showing expression of CD4 and CD8 in the thymus and spleen of ES cell-derived mice. Thymocytes and splenocytes from week old control and CD8 RNAi (pLL 3.7 CD8) ES cell-derived mice were harvested and stained for CD4 and CD8 expression.

FIG. 22A shows flow cytometric analysis of EGFP expression in cells infected with an EGFP-expressing lentiviral vector in which the promoter and EGFP coding sequences are floxed. The solid purple peaks represent uninfected cells. The population of cells expressing EGFP is shown with a green line.

FIG. 22B shows flow cytometric analysis of EGFP expression in cells infected with an EGFP-expressing lentiviral vector 10 days after induction of Cre expression. The solid purple peaks represent uninfected cells. The population of cells expressing EGFP is shown with a green line.

FIG. 22C shows a direct flow cytometric comparison between pLL3.7 infected D7 cells before (green line) and after (pink line) Cre delivery.

FIG. 23 shows flow cytometric analysis of CD8 expression in T cells transfected with transfer plasmids that direct expression of either an shRNA targeted to CD8 or an irrelevant stem-loop sequence, demonstrating silencing of CD8 by the CD8 shRNA. GFP expression is on the x-axis, and CD8 expression is on the y-axis. The upper panel shows lack of GFP expression in untransfected cells. The middle panel shows CD8 expression in GFP⁺ cells transfected with a transfer plasmid targeted to an unrelated sequence. The lower panel shows reduced CD8 expression in GFP⁺ cells transfected with a transfer plasmid targeted to CD8.

FIG. 24 shows flow cytometric analysis of expression of transfected human CD8 in wild type ES cells or ES cells infected with a mouse CD8 shRNA virus, demonstrating that the mouse CD8 shRNA specifically silences mouse CD8 and not human CD8.

FIG. 25 is a Northern blot showing that higher expression levels of CD8 shRNA in cells that did (left) versus cells that did not (right) exhibit silencing of CD8 following infection with a mouse CD8 shRNA virus.

### DEFINITIONS

The term defective as used herein refers to a nucleic acid that is not functional with regard to either (i) encoding its gene product or (ii) serving as a signaling sequence. For example, a defective env gene sequence does not encode a functional Env protein; a defective packaging signal will not facilitate the packaging of a nucleic acid molecule that includes the defective signal. A nucleic acid may be defective for some but not all of its functions. For example, a defective LTR may fail to promote transcription of downstream sequences while still retaining the ability to direct integration. Nucleic acid sequences may be made defective by any means known in the art, including by mutagenesis, by the deletion of some or all of the sequence, by inserting a heterologous sequence into the nucleic acid sequence, by placing the sequence out-of-frame, or by otherwise blocking the sequence. Defective sequences may also occur naturally, i.e., without human intervention, such as by mutation, and may be isolated from viruses in which they arise. Proteins that are encoded by a defective nucleic acid and are therefore not functional may be referred to as defective proteins. It is to be understood that the term “defective” is relative. In other words, the function need not be completely eliminated but is typically substantially reduced relative to the comparable wild type function. Generally, a defective sequence exhibits less than approximately 10% of the function of the comparable wild type sequence, preferably less than approximately 5% of the function of the comparable wild type sequence, yet more preferably less than approximately 2%, less than approximately 1%, less than approximately 0.5%, or approximately 0%, i.e., below the limits of detection.

The terms deleted or deletion are used herein in accordance with their standard usage in the art, i.e., meaning either total removal of the specified segment or the removal of a sufficient portion of the specified segment to render the segment inoperative or nonfunctional with respect to at least one of its functions.

The term heterologous as used herein in reference to a nucleic acid, refers broadly to a first nucleic acid that is inserted into a second nucleic acid such as a plasmid or vector. In particular, the term refers to a nucleic acid that is not naturally present in the wild type version of a virus-based vector or plasmid that is used to deliver the sequence into a cell. The term also refers to a nucleic acid that is introduced into a cell, tissue, organism, etc., by artificial means including, but not limited to, transfection, transformation, or infection with a viral vector. Generally the nucleic acid is either not naturally found in the cell, tissue, or organism or, if naturally found therein, its expression is altered by introduction of the additional copy of the nucleic acid (e.g., if the introduced copy is under the control of a different promoter than the naturally occurring copy). The term is also used to refer to a protein encoded by such a nucleic acid sequence. If a heterologous sequence is introduced into a cell or organism, the sequence is considered heterologous to the progeny of such a cell or organism.

The term hybridize, as used herein, refers to the interaction between two complementary nucleic acid sequences. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6×sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5 M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Infectious, as used herein in reference to a recombinant virus or viral particle, indicates that the virus or viral particle is able to enter cells in a manner substantially similar or identical to that of a wild type virus and to perform at least one of the functions associated with infection by a wild type virus, e.g., release of the viral genome in the host cell cytoplasm, entry of the viral genome into the nucleus, reverse transcription and integration of the viral genome into the host cell's DNA. It is not intended to indicate that the virus or viral particle is capable of undergoing replication or of completing the viral life cycle. The terms “viral particle” and “virus” are frequently used interchangeably herein. For example, the phrase “production of virus” may refer to production of viral particles and is not intended to indicate that wild type or replication competent virus is produced.

Isolated, as used herein, means 1) separated from at least some of the components with which it is usually associated in nature; 2) prepared or purified by a process that involves the hand of man; and/or 3) not occurring in nature.

Operably linked, as used herein, refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Purified, as used herein, means separated from many other compounds or entities, e.g., compounds or entities with which it normally occurs in nature. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term regulatory sequence is used herein to describe a region of nucleic acid sequence that directs, enhances, or inhibits the expression (particularly transcription, but in some cases other events such as splicing or other processing, translation, etc.) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc.

A short, interfering RNA (siRNA) comprises an RNA duplex that is approximately 19 basepairs long and optionally further comprises one or two single-stranded overhangs or loops. An siRNA may be formed from two RNA strands that hybridize together, or may alternatively be generated from a single RNA strand that includes a self-hybridizing portion. When siRNAs include one or more free strand ends, it is generally preferred that free 5′ ends have phosphate groups, and free 3′ ends have hydroxyl groups. siRNAs include a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

The term short hairpin RNA refers to an RNA molecule comprising at least two complementary portions hybridized or capable of hybridizing to form a double-stranded structure sufficiently long to mediate RNAi (typically at least 19 base pairs in length), and at least one single-stranded portion, typically between approximately 1 and 10 nucleotides in length that forms a loop. As described further below, shRNAs are thought to be processed into siRNAs by the conserved cellular RNAi machinery. Thus shRNAs are precursors of siRNAs and are similarly capable of inhibiting expression of a target transcript.

The phrase structural protein as used herein refer to the proteins which are required for encapsidation (e.g., packaging) of a retroviral or lentiviral genome, and include Gag, Pol and Env.

The term subject, as used herein, refers to any individual to whom a lentiviral vector of the invention is delivered for any purpose. Preferred subjects are mammals, particularly rodents (e.g., mice and rats), domesticated mammals (e.g., dogs, cats, etc.), primates, or humans.

An siRNA or shRNA or an siRNA or shRNA sequence is considered to be targeted to target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 17, more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. Since the effect of targeting a transcript is to reduce or inhibit expression of the gene that directs synthesis of the transcript, an siRNA or shRNA targeted to a transcript is also considered to target the gene that directs synthesis of the transcript even though the gene itself (i.e., genomic DNA) is not thought to interact with the siRNA, shRNA, or components of the cellular silencing machinery. Thus as used herein, an siRNA or shRNA that targets a gene is understood to target a transcript whose synthesis is directed by the gene.

The term vector is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Useful viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid. Thus the term viral vector may refer either to a virus or viral particle capable of transferring a nucleic acid into a cell or to the transferred nucleic acid itself. In particular, the terms “lentiviral vector”, “lentiviral expression vector” may be used to refer to lentiviral transfer plasmids and/or lentiviral particles of the invention as described below.

### DETAILED DESCRIPTION OF CERTAIN PREFERRED EMBODIMENTS OF THE INVENTION

Retroviruses and Retroviral Vectors

The retrovirus family consists of a group of viruses with a diploid RNA genome that is reverse transcribed during the viral life cycle to yield a double-stranded DNA intermediate that stably integrates into the chromosomal DNA of a host cell. The integrated DNA intermediate is referred to as a provirus. As used herein, a provirus is “derived from” a virus or viral particle that delivers the nucleic acid from which the proviral DNA is reverse transcribed to the cytoplasm of the cell. The retroviral genome and proviral DNA include three genes referred to as gag, pol, and env, flanked by two long terminal repeat sequences (LTRs). The 5′ and 3′ LTRs contain elements that promote transcription (promoter-enhancer elements) and polyadenylation of viral RNA. The LTRs also include additional cis-acting sequences required for viral replication. In addition, the viral genome includes a packaging signal referred to as psi (Ψ) that is necessary for encapsidation (packaging) of the retroviral genome. As used herein, a packaging signal or psi sequence is any sequence sufficient to direct packaging of a nucleic acid whose sequence comprises the packaging signal. This includes naturally occurring psi sequences and also engineered variants thereof.

Briefly, the normal infective cycle begins when the virus attaches to the surface of a susceptible cell through interaction with one or more cell surface receptors. The virus fuses with the cell membrane, and the viral core is delivered to the cytoplasm, where the viral matrix and capsid become dismantled, releasing the viral genome. Viral reverse transcriptase copies the RNA genome into DNA, which moves into the nucleus, where its integration into host cell DNA is catalyzed by the viral integrase enzyme.

Once integrated into a host genome, viral DNA can remain dormant for long periods of time. When activated, the viral DNA is transcribed by host cell RNA polymerase. The resulting transcript is both a genome for a new virion and a transcript from which viral gag and gag-pol polyproteins are synthesized. These polyproteins are later processed into the matrix (MA), capsid (CA), and nucleocapsid (NC) proteins (in the case of gag), or the matrix, capsid, protease (PR), reverse transcriptase (RT), and integrase (INT) proteins (in the case of gag-pol). The full-length viral RNA transcript also yields transcripts that act as templates for synthesis of other viral proteins including envelope glycoproteins and, in the case of lentiviruses, a number of regulatory proteins via various splicing events. Newly made Gag and Gag-Pol polyproteins associate with one another, with complete viral genomes, and with envelope proteins in the cell membrane so that a new viral particle begins to assemble at the membrane. As assembly continues, the structure extrudes from the cell, thereby acquiring a lipid coat punctuated with envelope glycoproteins. Further discussion of the retroviral life cycle and features and descriptions of retrovirus classification and taxonomy may be found in Coffin, J., et al. (eds.), Retroviruses, Cold Spring Harbor Laboratory Press, Cold Spring Harbor, 1997, and in Fields, B., et al., Fields' Virology, 4^{th}. ed., Philadelphia: Lippincott Williams and Wilkins; ISBN: 0781718325, 2001. See also the Web site having URL www.ncbi.nlm.nih.gov/ICTVdb/ICTVdB, accessed Oct. 11, 2002, providing a classification and information about viruses, of which retroviruses are entry 61 and lentiviruses are entry 61.0.6.

The ability of retroviruses to enter host cells and to mediate the integration of heterologous nucleic acid sequences into the cellular genome (transduction) has led to their widespread use for in vitro and in vivo transfer and expression of nucleic acids, a process often referred to as gene transfer. However, the heterologous nucleic acid need not be a gene and need not encode a protein. As used herein, the term “gene transfer” refers to transfer of any nucleic acid. A transferred nucleic acid is “expressed” in a cell if the introduction of the nucleic acid into the cell results, either directly or indirectly (such as via reverse transcription, integration, transcription, and, in some cases, translation) in the presence of an expression product of the nucleic acid (e.g., an RNA transcript and/or a polypeptide) within the cell.

Advantages of retroviral vector systems include: (i) efficient entry of genetic material (the vector genome) into cells; (ii) an efficient process of entry into the target cell nucleus; (iii) relatively high levels of gene expression in many settings; (iv) minimal pathological effects on target cells in the case of many retroviruses; and (v) the potential to target particular cellular subtypes through control of the vector-target cell binding and tissue-specific control of gene expression (e.g., using tissue-specific promoters and/or enhancers).

In using a retrovirus for gene transfer, a foreign (not part of the wild type virus) sequence (e.g., a gene of interest) may be inserted into the retroviral genome in place of wild type retroviral sequences. When the retrovirus delivers its genome to a cell, the foreign sequence is also introduced into the cell and may then be integrated into the host's cellular DNA as in the case of a wild type retroviral genome. The sequence may then be transcribed by the host cell's transcriptional machinery. If the sequence includes a coding region, translation of the sequence within the host results in expression of the encoded protein by the host cell. The features described above have made retroviral vectors particularly attractive for gene therapy although they may be used in numerous other applications as described below.

In order to improve their safety, many recombinant retroviruses designed for gene transfer are replication defective, i.e., the genome does not encode functional forms of all the proteins necessary for the complete infective cycle. For example, sequences encoding the structural proteins may be mutated or deleted. In particular, part or all of the sequence encoding the structural proteins may be replaced by a different nucleic acid sequence, i.e., a nucleic acid sequence that is to be introduced into a target cell. However, the packaging signal remains intact. The nucleic acid sequence may include a promoter or its transcription may be under control of the viral LTR promoter-enhancer. In order to produce infectious viral particles that can be used to deliver the recombinant genome to cells, the required viral proteins are provided in trans. This may be accomplished using a variety of approaches as further described below.

Lentiviruses and Lentiviral Vectors

Lentiviruses are a family of retroviruses that differ from the simple retroviruses described above in that their genome includes any of a variety of genes in addition to Gag, Pol, and Env and may also include various regulatory elements. The additional genes encode typically include regulatory proteins such as Vif, Vpr, Vpu, Tat, Rev, and Nef. (For a discussion of various transcripts present at different times during the life cycle of HIV, see, for example, Kim et al., J. Virol. 63:3708, 1989, incorporated herein by reference). Further discussion of the lentiviral life cycle and features and descriptions of lentivirus classification and taxonomy may be found in Coffin, J., et aL (eds.), Retroviruses, Cold Spring Harbor Laboratory Press, Cold Spring Harbor, 1997, and in Fields, B., et al., Fields' Virology, 4^{th}. ed., Philadelphia: Lippincott Williams and Wilkins; ISBN: 0781718325, 2001.

The fact that retroviruses cannot effectively direct integration into the genome of nondividing cells has limited their use for introducing genes into many important targets such as liver, skeletal muscle, heart, brain, retina, and various cells of the hematopoietic system. In contrast, lentiviruses are able to productively infect and transduce nondividing cells, which has motivated the development of lentiviral vectors for gene transfer^{20,21}. For example, lentiviruses are able to infect resting T cells, dendritic cells, and macrophages. Lentiviral vectors can also transfer genes to hematopoietic stem cells with a superior gene transfer efficiency and without affecting the repopulating capacity of these cells. Lentiviral vectors can also transduce liver, skeletal muscle, retina, and neuronal cells. See, e.g., Mautino and Morgan, AIDS Patient Care STDS 2002 January;16(1):11-26; Somia, N., et al. J. Virol. 74(9):4420-4424, 2000; Miyoshi, H., et al., Science 283: 682-686, 1999; U.S. Pat. No. 6,013,516, and references 21 and 24. In addition, lentiviruses display reduced susceptibility to developmental silencing relative to simple retroviruses (24). This feature enables their use for the creation of transgenic animals, which is impractical with simple retroviruses because developmental silencing results in low or undetectable levels of transgene expression.

As mentioned above, to enhance safety recombinant retroviruses and lentiviruses designed for gene transfer are typically replication defective, i.e., the genome does not encode functional forms of all the proteins necessary for the complete infective cycle. The necessary proteins are therefore provided in trans. According to one approach, these proteins are provided by a packaging cell that has been engineered to produce the proteins. Methods for preparing packaging cell lines that express retrovirus proteins are well known in the art (See, e.g., U.S. Pat. No. 4,650,764 to Temin et al., U.S. Pat. No. 5,955,331 to Danos, et al., Sheridan et al., Molecular Therapy 2(3):262-275, September, 2000). Known packaging cell lines include Ψ2, PA137, and PA12, among others.

In the absence of a nucleic acid sequence containing appropriate packaging signals, the packaging cell produces empty virions. When a nucleic acid sequence containing appropriate packaging signals is present within the packaging cell (as may be achieved by either stably or transiently transfecting the cell with a construct capable of directing transcription of such a sequence), the sequence can be packaged, yielding infectious viral particles. The resulting cell is referred to as a producer cell.

In the context of certain embodiments of the present invention, a packaging cell will comprise a host cell containing packaging-signal defective nucleic acid sequence(s) coding for retroviral protein(s). The cell is thus able to produce retroviral protein(s) but unable to produce replication-competent infectious virus. Packaging cells may be created by transfecting a host cell (e.g., a human 293T cell) with one or more nucleic acid sequences encoding such protein(s) according to known procedures. Any suitable combination of expression cassettes capable of driving synthesis of the required proteins is sufficient. Typically the packaging cell line contains (i) a modified retroviral genome encoding functional Gag and Gag-Pol polyproteins but unable to produce functional envelope protein; and (ii) a sequence encoding an envelope protein.

The various proteins need not all originate from the same viral species. For example, the Gag and Pol proteins may be derived from any of a wide variety of retroviruses or lentiviruses. According to certain preferred embodiments of the invention the gag and pol proteins are derived from a lentivirus. According to certain embodiments of the invention the gag and pol proteins are derived from HIV. Many different types of host cell may be used, provided that the cells are permissive for transcription from the promoters employed. Suitable host cells include, for example, 293 cells and derivatives thereof such as, 293.T, 293FT (Invitrogen), 293F, etc., NIH3T3 cells, etc. In general, any mammalian cell that supports transfection and can be grown in sufficient quantities can be used. One of ordinary skill in the art will be able to select appropriate host cells.

Although an envelope derived from the same retrovirus or lentivirus from which the other viral proteins are derived can be used (homologous envelope) the use of a nonhomologous envelope protein such as the VSV G glycoprotein significantly reduces or eliminates the possibility of generating wild-type virus during vector manufacturing or after introduction of the vectors into host cells. Thus one useful class of lentiviral vectors consists of replication-defective, hybrid viral particles made from the core proteins and enzymes of a lentivirus and the envelope of a different virus such as the vesicular stomatitis virus (VSV) or the Moloney leukemia virus.

Safety considerations prompted development of alternative approaches to the production of recombinant retroviral and lentiviral particles capable of infecting and transducing cells. According to an approach described in U.S. Pat. No. 6,013,516 and also in references 19 and 20, three different constructs may be used to produce the recombinant lentiviral particles. Two of these constructs provide packaging functions, one containing sequences encoding the core proteins and enzymes of the lentivirus and the other containing sequences encoding the envelope protein of a different related or unrelated virus.

The third construct, referred to herein as a transfer construct, transfer vector, or transfer plasmid, includes a cloning site for insertion of a heterologous nucleic acid (i.e., a sequence not derived from the lentivirus) in addition to the cis-acting viral sequences that are necessary for certain aspects of the viral life cycle such as encapsidation, reverse transcription, and integration.

The three plasmid system, which does not require helper virus, and use of a heterologous envelope improve the safety of the vector by reducing the likelihood that a replication-competent recombinant could be generated. In addition, removal of various non-essential cis-acting sequences and the discovery that sequences encoding certain viral proteins can be removed while still allowing efficient gene transfer further contributes to the safety of this system. These advances are reviewed in reference 21 and articles listed therein, all of which are incorporated herein by reference.

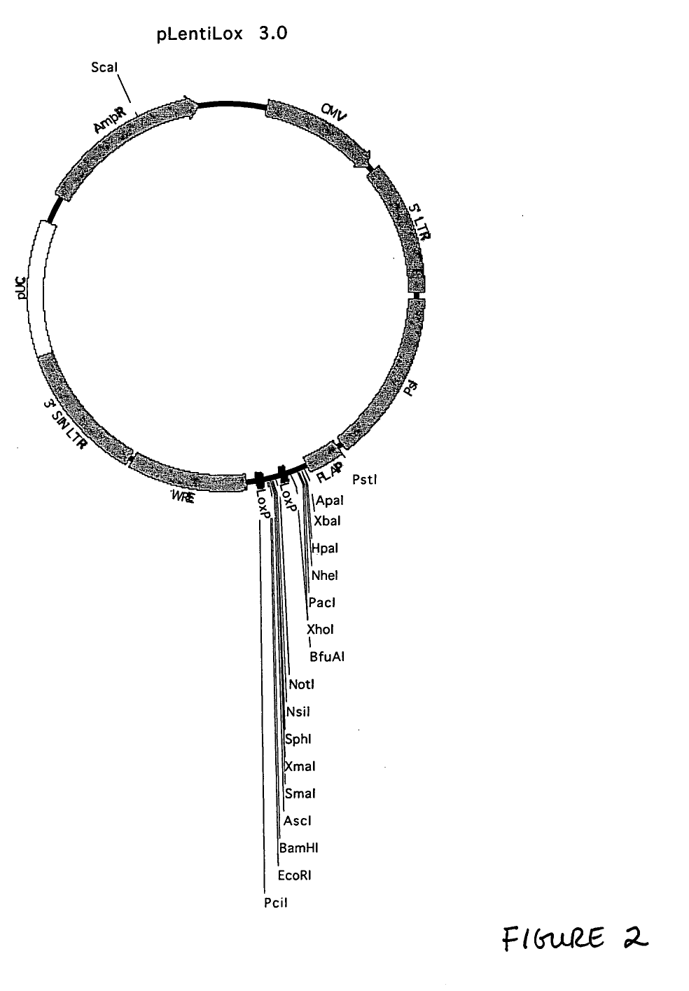

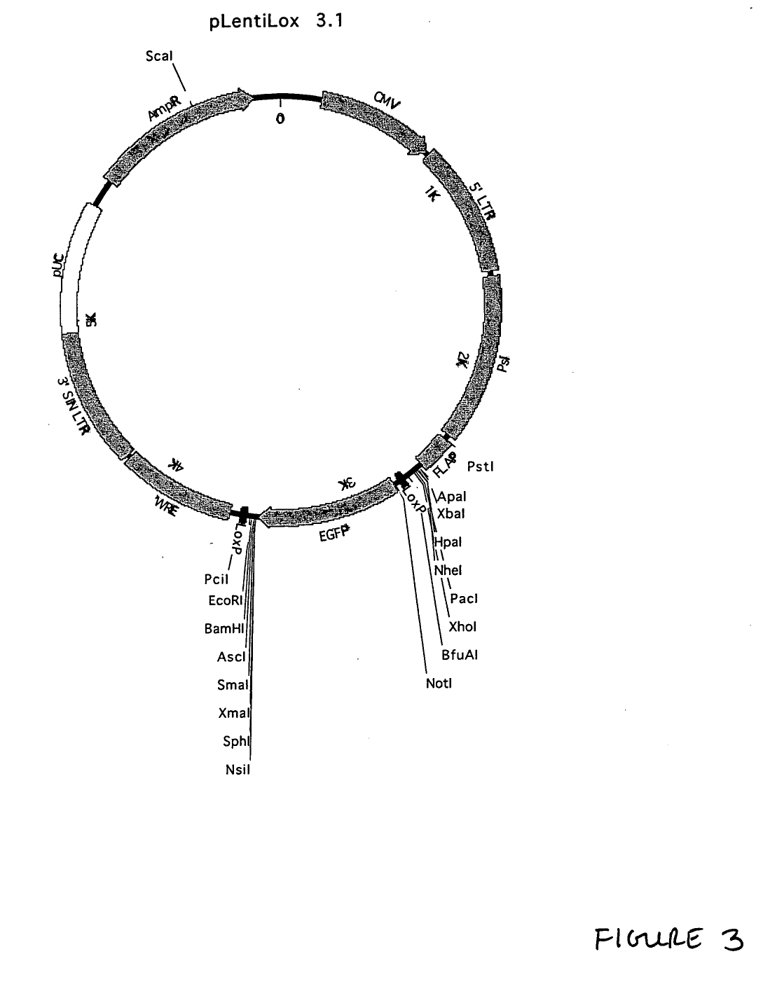

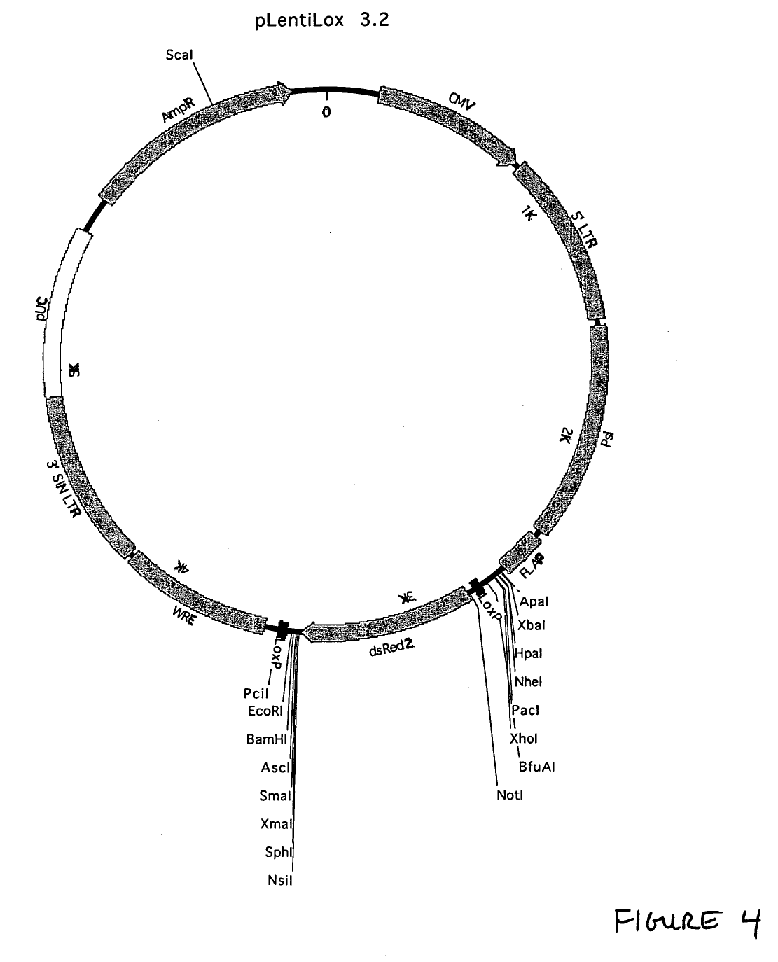

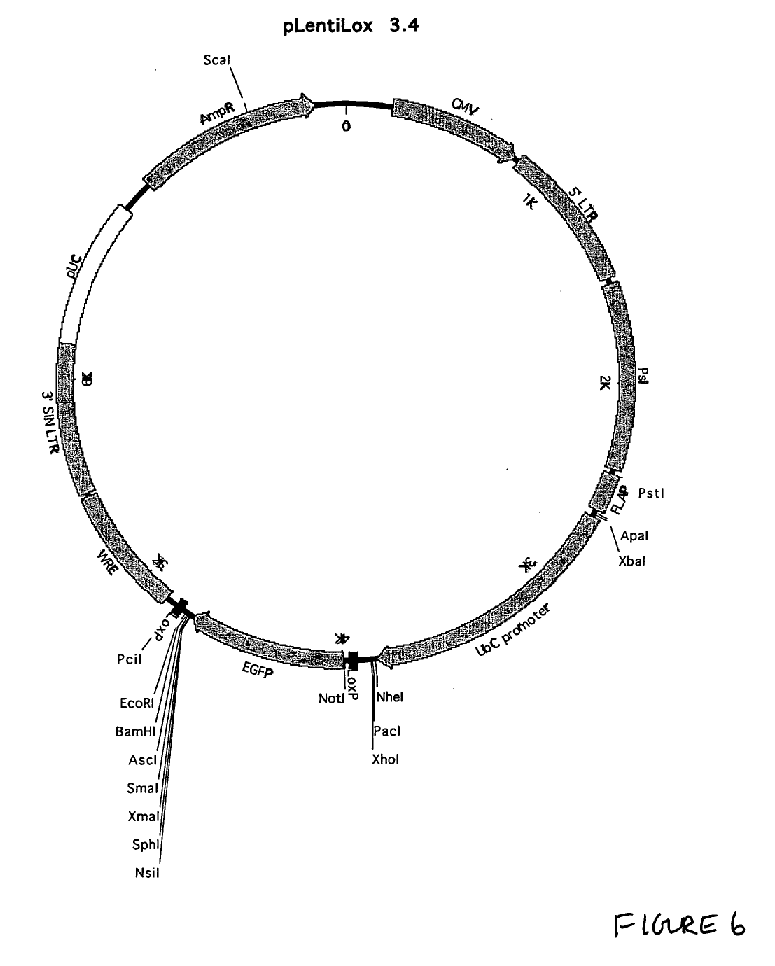

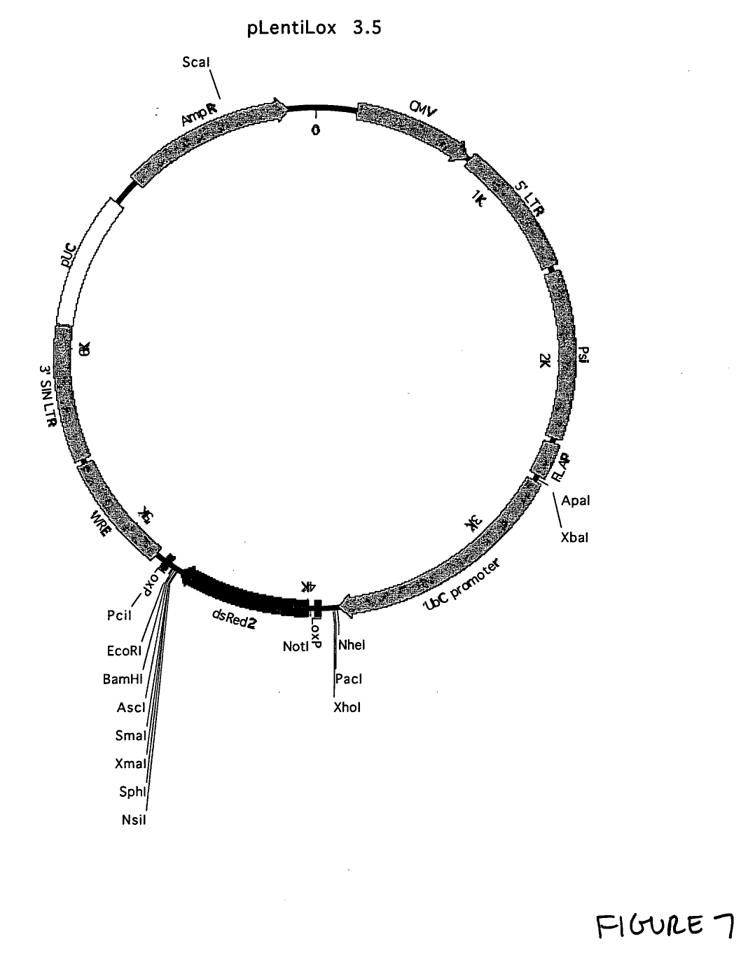

The present invention provides new lentiviral transfer plasmids, new replication-defective lentiviruses, and new lentiviral expression systems. Maps of exemplary lentiviral transfer constructs of the invention are provided in FIGS. 2 through 9 and corresponding sequences are provided as SEQ ID NOS: 2 through 9. However, the invention is not limited to these specific embodiments. FIG. 2 shows a map of one of the transfer plasmids of the invention in which nucleotide 0 is indicated. For purposes of description, nucleotides are numbered in a clockwise direction with reference to nucleotide 0, and elements having lower nucleotide numbers are considered 5′ to elements having higher nucleotide numbers. Thus, for example, the CMV element is 5′ to all other elements shown. Note that various elements depicted in the maps are not shown to scale. Also, the presence of a particular element on a map is not intended to indicate that the entire element is necessarily present. For example, according to certain embodiments of the invention a portion of the 5′ LTR is deleted.

According to certain embodiments of the invention the lentiviral transfer plasmids are HIV-based lentiviral transfer plasmids: As used herein, a lentiviral plasmid is said to be “based on” a particular lentivirus species (e.g., HIV-1) or group (e.g., primate lentivirus group) if at least 50% of the lentiviral sequences found in the plasmid are derived from a lentivirus of that particular species or group, alternately, if the transfer plasmid displays greater identity or homology to a lentivirus of that particular species or group than to other known lentiviruses. Thus a HIV-based lentiviral transfer plasmid is a transfer plasmid in which at least 50% of the lentiviral sequences are derived from (i.e., originate from), either HIV-1 or HIV-2 or, alternately, if the transfer plasmid displays greater identity or homology to HIV-1 or HIV-2 than to other known lentiviruses. In cases where the origin of any given sequence is unknown, the likelihood that it is derived from a particular lentivirus may be determined by sequence comparison using, e.g., programs such as BLAST, BLASTNR, or CLUSTALW (or variations thereof) in a comprehensive database such as GenBank, Unigene, etc., can be performed using, e.g., default parameters and matrices (e.g., BLOSUM substitution matrix). (BLAST is described in Altschul, SF, et al., Basic local alignment search tool, J. Mol. Biol., 215(3): 403-410, 1990, Altchul, SF and Gish, W, Methods in Enzymology.

The invention provides a lentiviral transfer plasmid whose sequence comprises a nucleic acid sequence including (i) a functional packaging signal; (ii) a multiple cloning site (MCS); and (iii) at least one additional element selected from the group consisting of: a second MCS, a second MCS into which a heterologous promoter or promoter-enhancer is inserted, an HIV FLAP element, an expression-enhancing posttranscriptional regulatory element, a target site for a site-specific recombinase, and a self-inactivating (SIN) LTR. It is to be understood that the target site for a site-specific recombinase is in addition to any site(s) required for integration of the lentiviral genome. In other words, in those embodiments of the invention in which the additional element is a target site for a site-specific recombinase, the lentiviral transfer plasmid will typically also include target sites for the corresponding lentiviral integrase (which normally exist within the LTRs). In particular, the invention provides (1) a lentiviral transfer construct as described immediately above wherein the additional element is a second MCS; (2) a lentiviral transfer construct as described immediately above wherein the additional element is a second MCS in which a heterologous promoter or promoter-enhancer is inserted; (3) a lentiviral transfer construct as described immediately above wherein the additional element is an HIV FLAP element; (4) a lentiviral transfer construct as described immediately above wherein the additional element is an expression-enhancing posttranscriptional regulatory element such as the woodchuck hepatitis virus regulatory element (WRE); (5) a lentiviral transfer construct as described immediately above wherein the additional element is a recombination site for a site-specific recombinase; and (6) a lentiviral transfer construct as described immediately above wherein the additional element is a SIN LTR. The lentiviral transfer plasmid may also comprise one or more heterologous promoters, enhancers, or promoter-enhancers.

The invention further provides lentiviral transfer plasmids containing at least two, at least three, at least four, at least five, or all of these additional elements. In particular, the invention provides a lentiviral tranfer plasmid comprising a nucleic acid sequence that includes (i) a functional packaging signal; (ii) a multiple cloning site (MCS); (iii) a second MCS; (iv) a second MCS in which a heterologous promoter or a heterologous promoter-enhancer is inserted; (v) an HIV FLAP element; (vi) a WRE; (vii) two loxP sites; and a self-inactivating (SIN) LTR . The invention also encompasses lentiviral transfer plasmids as described above in which a heterologous nucleic acid is inserted at a site within an MCS. It will be appreciated that insertion of such a sequence separates the MCS into two parts.

According to preferred embodiments of the invention the transfer plasmid includes the cis-acting sequence elements required to support reverse transcription of a lentiviral genome and also the cis-acting sequence elements necessary for the packaging and integration of a lentiviral genome. These sequences typically include the Psi (Ψ) packaging sequence, reverse transcription signals, integration signals, promoter or promoter/enhancer, polyadenylation sequence, tRNA binding site, and origin for second strand DNA synthesis. According to certain embodiments of the invention the transfer plasmid contains a Rev Response Element (RRE) such as that located at positions 7622-8459 in the HIV NL4-3 genome (Genbank accession number AF003887). Of course RREs from other strains of HIV can also be used. Such sequences are readily available from Genbank or from the database having URL hiv-web.lanl.gov/content/index. According to certain embodiments of the invention the transfer plasmid contains a 5′ HIV R-U5-del gag element such as that located at positions 454-1126 in the HIV NL4-3 genome. According to preferred embodiments of the invention the transfer plasmid contains a sequence encoding a selectable marker such as the ampicillin resistance gene (AmpR) and an origin of replication that allows the plasmid to replicate within bacterial cells, such as the pUC origin. Various features and elements mentioned above (and others) are more fully described in the following sections.

Lentiviral Genome Sequences.

The lentiviral transfer plasmids may include lentiviral sequences derived from any of a wide variety of lentiviruses including, but not limited to, primate lentivirus group viruses such as human immunodeficiency viruses HIV-1 and HIV-2 or simian immunodeficiency virus (SIV); feline lentivirus group viruses such as feline immunodeficiency virus (FIV); ovine/caprine immunodeficieny group viruses such as caprine arthritis encephalitis virus (CAEV); bovine immunodeficiency-like virus (BIV); equine lentivirus group viruses such as equine infectious anemia virus; and visna/maedi virus. It will be appreciated that each of these viruses exists in multiple variants or strains.

According to certain preferred embodiments of the invention most or all of the lentiviral sequences are derived from HIV-1. For example, according to certain embodiments of the invention the lentiviral backbone of the transfer plasmids is derived from an HIV-1-based transfer plasmid such as that described in reference 29 or derivatives thereof such as those described in reference 24. However, it is to be understood that many different sources of lentiviral sequences can be used, and numerous substitutions and alterations in certain of the lentiviral sequences may be accommodated without impairing the ability of the transfer plasmid to perform the functions described herein, and such variations are within the scope of the invention. The ability of any particular lentiviral transfer plasmid to transfer nucleic acids and/or to generate a lentiviral particle capable of infecting and transducing cells in the presence of the required viral proteins may readily be tested by methods known in the art, some of which will be evident from the Examples.

Unique Restriction Sites and Multiple Cloning Sites.

The invention provides new lentiviral transfer plasmids incorporating sites for a variety of different restriction enzymes. In particular, the invention provides lentiviral transfer constructs including one or more multiple cloning sites (MCS), e.g., one MCS or two MCSs. As is well known in the art, a multiple cloning site, also referred to as a polylinker, or polycloning site, is a cluster of cloning sites such that many restriction enzymes operate within the site. A cloning site as used herein is a known sequence, preferably the only one on the plasmid, (i.e., it is a unique sequence on the plasmid) upon which a restriction enzyme operates to linearize or cut the plasmid. Restriction sites for numerous restriction enzymes are known in the art and are listed, for example, in the catalogs of various manufacturers such as New England Biolabs, Promega, Beoringer-Ingelheim, etc. For purposes of the present invention a restriction site is unique if it is recognized as such in the art or, alternately, if the enzyme displays at least a 5-fold greater likelihood of cutting at the unique site than at any other site in the plasmid under standard digestion conditions.

Typically an MCS is less than approximately 100 nucleotides in length (measured from the most 5′ nucleotide in the most 5′ restriction site to the most 3′ nucleotide in the most 3′ restriction site, and including both of these nucleotides) and contains at least 4 unique restriction sites. According to certain embodiments of the invention an MCS is less than approximately 100 nucleotides in length. According to certain embodiments of the invention an MCS is less than approximately 75 nucleotides in length. According to certain embodiments of the invention an MCS is less than approximately 50 nucleotides in length. According to certain embodiments of the invention the transfer plasmid comprises an MCS containing at least 5 unique restriction sites. According to other embodiments of the invention the transfer plasmid comprises an MCS containing at least 6 unique restriction sites. According to yet other embodiments of the invention the transfer plasmid comprises an MCS containing at least 7 unique restriction sites. According to yet other embodiments of the invention the transfer plasmid comprises an MCS containing at least 8 unique restriction sites. According to yet other embodiments of the invention the transfer plasmid comprises an MCS containing at least 9 unique restriction sites. According to yet other embodiments of the invention the transfer plasmid comprises at least two MCSs, each of which contains at least 7 unique restriction sites. The invention provides a lentiviral transfer plasmid containing an MCS that includes a site for a restriction enzyme that leaves a blunt end after cutting. The invention further provides a lentiviral transfer plasmid containing an MCS that includes a restriction site that has an 8 bp recognition sequence.

The invention provides a lentiviral transfer plasmid having unique restriction sites for at least 4 enzymes selected from the group consisting of NotI, Apal, XhoI, XbaI, HpaI, NheI, PacI, NsiI, SphI, Sma/Xma, AccI, BamHI, and SphI. The invention further provides a lentiviral transfer plasmid having unique restriction sites for at least 5, at least 6, at least 7, at least 8, at least 9, at least 10, at least 11, at least 12, or at least 13 enzymes selected from the group consisting of NotI, ApaI, XhoI, XbaI, HpaI, NheI, PacI, NsiI, SphI, Sma/Xma, AccI, BamHI, and SphI. The invention further provides collections of two or more of any of the lentiviral transfer plasmids described above. According to certain embodiments of the invention any of the lentiviral transfer plasmids described above are HIV-based transfer plasmids.

HIV FLAP Element.

According to certain embodiments of the invention the transfer plasmid includes an HIV FLAP element. This sequence contains structural elements associated with the process of reverse transcription and encompasses the central polypurine tract and central termination sequences (cPPT and CTS). As described in Zennou, et al., Cell, 101, 173, (2000), during HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus.

Expression-Stimulating Posttranscriptional Regulatory Element.

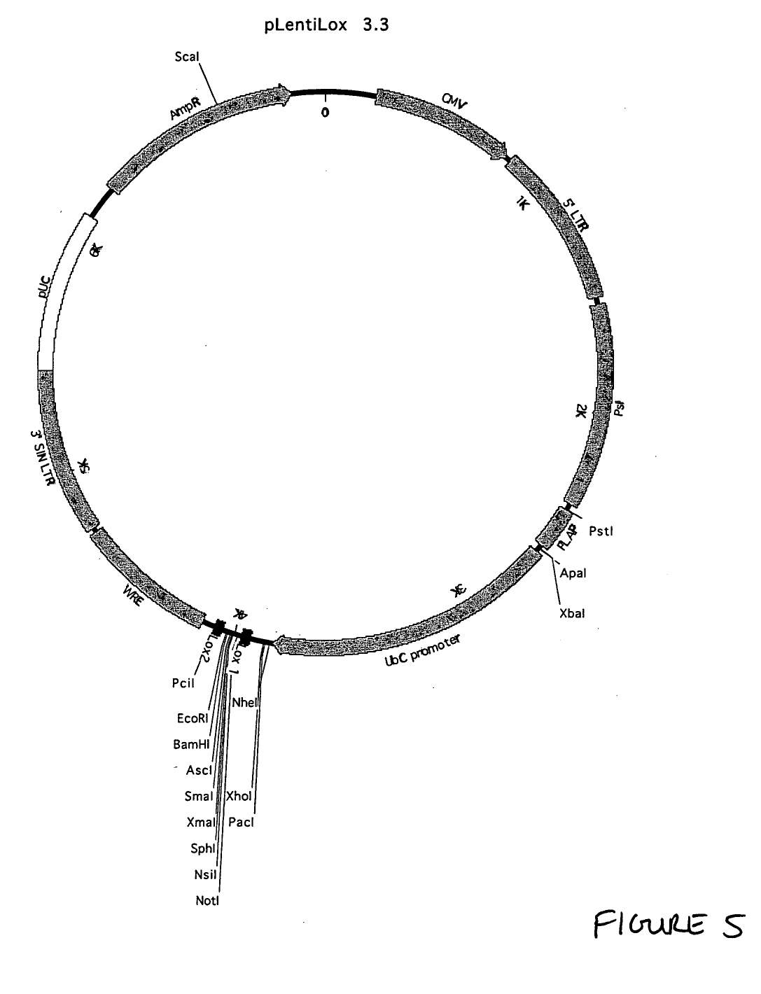

The invention provides lentiviral transfer plasmids comprising any of a variety of posttranscriptional regulatory elements characterized in that their presence within a transcript increases expression of the heterologous nucleic acid at the protein level. According to certain embodiments of the invention the posttranscriptional regulatory element is the woodchuck hepatitis virus regulatory element (WRE) as described in Zufferey, R., et al., J. Virol., 73, 2886, 1999. Other posttranscriptional processing elements that may be used include the posttranscriptional processing element present within the genome of various viruses such as that present within the thymidine kinase gene of herpes simplex virus (Liu, X., and J. E. Mertz. Genes Dev. 9:1766-1780, 1995), and the posttranscriptional regulatory element (PRE) present in hepatitis B virus (HBV) (Huang, Z. M., and T. S. Yen, Mol. Cell. Biol. 5:3864-3869, 1995). According to the invention the posttranscriptional regulatory element is positioned so that a heterologous nucleic acid inserted into the transfer plasmid in the 5′ directly from the element will result in production of a transcript that includes the posttranscriptional regulatory element at the 3′ end. FIG. 2 shows an example of a transfer plasmid incorporating the WRE downstream of sites for insertion of one or more heterologous nucleic acid sequences. FIG. 6 shows an example of a transfer plasmid in which a heteologous nucleic acid encoding EGFP has been inserted in the 5′ direction from the WRE and the ubiqutin C (UbC) promoter has been inserted upstream of the sequence encoding EGFP. This configuration results in synthesis of a transcript whose 5′ portion comprises EGFP coding sequences and whose 3′ portion comprises the WRE sequence.

Long Terminal Repeats.

According to certain embodiments of the invention the transfer plasmid includes a self-inactivating (SIN) LTR (29). As is known in the art, during the retroviral life cycle, the U3 region of the 3′ LTR is duplicated to form the corresponding region of the 5′ LTR in the course of reverse transcription and viral DNA synthesis. Creation of a SIN LTR is achieved by inactivating the U3 region of the 3′ LTR (preferably by deletion of a portion thereof as described in reference 29). The alteration is transferred to the 5′ LTR after reverse transcription, thus eliminating the transcriptional unit of the LTRs in the provirus, which should prevent mobilization by replication competent virus. An additional safety enhancement is provided by replacing the U3 region of the 5′ LTR with a heterologous promoter to drive transcription of the viral genome during production of viral particles. Appropriate promoters include, e.g., the CMV promoter. Preferred promoters are able to drive high levels of transcription in a Tat-independent manner. This replacement further reduces the possibility of recombination to generate replication-competent virus because there is no complete U3 sequence in the virus production system. Thus in certain embodiments of the invention the transfer plasmid includes a self-inactivating (SIN) 3′ LTR. In certain embodiments of the invention the transfer plasmid includes a 5′ LTR in which the U3 region is replaced with a heterologous promoter. The heterologous promoter drives transcription during transient transfection but after reverse transcription it gets replaced by a copy of U3 from the 3′ LTR, which in the case of a SIN LTR contains a deletion that makes it unable to drive transcription. Thus all transcription is driven by the internal promoter after integration.

According to certain embodiments of the invention one or both LTRs contain sequences that can be used to introduce insulator sequences into the vectors. In general, insulators are elements that can help to preserve the independent function of genes or transcription units embedded in a genome or genetic context in which their expression may otherwise be influenced by regulatory signals within the genome or genetic context. See, for example, Burgess-Beusse B, et al., Proc. Natl. Acad. Sci. published Aug. 1, 2002, 10.1073/pnas.162342499 and Zhan HC, et al., Hum Genet, November;109(5):471-8, 2001. In the context of the present invention, insulators “protect” the lentivirus-expressed sequences from integration site effects, which are mediated by cis-acting elements present in genomic DNA, and lead to deregulated expression of transferred sequences. The invention provides transfer plasmids in which an insulator sequence is inserted into one or both LTRs.

Heterologous Promoters and Promoter/Enhancers.

Any of a wide variety of heterologous promoter and promoter/enhancer elements may be included in the transfer plasmids and used to direct transcription of a heterologous nucleic acid sequence in cells infected with the recombinant lentiviral particles of the invention or cells into which the transfer plasmids of the invention have been introduced, e.g., by transfection. According to certain embodiments of the invention the transfer plasmids and lentiviral particles include a single heterologous promoter. In other embodiments two or more heterologous promoters are included. The promoters may be in the same or in opposite orientation.

One of ordinary skill in the art will readily be able to selet appropriate promoters depending upon the particular application. For example, sometimes it will be desirable to achieve constitutive, non-tissue specific, high level expression of a heterologous nucleic acid sequence. For such purposes viral promoters or promoter/enhancers such as the SV40 promoter, CMV promoter or promoter/enhancer, etc., may be employed. Mammalian promoters such as the beta-actin promoter, ubiquitin C promoter, elongation factor 1α promoter, tubulin promoter, etc., may also be used. If the plasmids are to be used in non-mammalian cells, appropriate promoters for such cells should be selected.

It may be desirable to achieve cell type specific or tissue-specific expression of a heterologous nucleic acid sequence (e.g., to express a particular heterologous nucleic acid in only a subset of cell types or tissues or during specific stages of development), tissue-specific promoters may be used. For example, it may be desirable to achieve conditional expression in the case of transgenic animals or for therapeutic applications, including gene therapy. As used herein, the term “tissue specific promoter” refers to a regulatory element (e.g., promoter, promoter/enhancer or portion thereof) that preferentially directs transcription in only a subset of cell or tissue types, or during discrete stages in the development of a cell, tissue, or organism. A tissue specific promoter may direct transcription in only a single cell type or in multiple cell types (e.g., two to several different cell types). Numerous tissue-specific promoters are known, and one of ordinary skill in the art will readily be able to identify tissue specific promoters (or to determine whether any particular promoter is a tissue specific promoter) from the literature or by performing experiments such as Northern blots, immunoblots, etc. in which expression of either an endogenous gene or a reporter gene operably linked to the promoter is compared in different cell or tissue types). For example, the nestin, neural specific enolase, NeuN, and GFAP promoters direct transcription in various neural or glial lineage cells; the keratin 5 promoter directs transcription in keratinocytes; the MyoD promoter directs transcription in skeletal muscle cells; the insulin promoter directs transcription in pancreatic beta cells; the CYP450 3A4 promoter directs transcription in hepatocytes. The invention therefore provides lentiviral transfer plasmids as described above comprising a tissue-specific promoter and methods of using the transfer plasmids and lentiviral particles derived therefrom to achieve cell type or tissue specific expression. Preferred promoters are active in mammalian cells. According to certain embodiments of the invention the tissue-specific promoter is specific for brain (e.g., neurons), liver (e.g., hepatocytes), pancreas, skeletal muscle (e.g., myocytes), immune system cells (e.g., T cells, B cells, macrophages), heart (e.g., cardiac myocytes), retina, skin (e.g., keratinocytes), bone (e.g., osteoblasts or osteoclasts), etc.

It may be desirable to achieve conditional expression of a heterologous nucleic acid sequence (e.g., to control expression of a particular heterologous nucleic acid by subjecting a cell, tissue, organism, etc., to a treatment or condition that causes the heterologous nucleic acid to be expressed or that causes an increase or decrease in expression of the heterologous nucleic acid), for which purpose a variety of inducible promoters and systems. In particular, it may be desirable to achieve conditional expression in the case of transgenic animals or for therapeutic applications, including gene therapy. See, e.g., Haviv Y S and Curiel D T, Adv Drug Deliv Rev, 53(2):135-54, 2001, describing approaches for achieving conditional gene expression in cancer cells. As used herein, “conditional expression” may refer to any type of conditional expression including, but not limited to: inducible expression; repressible expression; expression in cells or tissues having a particular physiological, biological, or disease state, etc. This definition is not intended to exclude cell type or tissue-specific expression, since the type of cell or tissue may also be considered a condition.

One approach to achieving conditional expression involves the use of inducible promoters. As used herein, the term “inducible promoter” refers to a regulatory element (e.g., a promoter, promoter/enhancer or portion thereof) whose transcriptional activity may be regulated by exposing a cell or tissue containing a nucleic acid sequence operably linked to the promoter to a treatment or condition that alters the transcriptional activity of the promoter, resulting in increased transcription of the nucleic acid sequence. For convenience, as used herein, the term “inducible promoter” also includes repressible promoters, i.e., promoters whose transcriptional activity may be regulated by exposing a cell or tissue containing a nucleic acid sequence operably linked to the promoter to a treatment or condition that alters the transcriptional activity of the promoter, resulting in decreased transcription of the nucleic acid sequence. Preferred inducible promoters are active in mammalian cells. Inducible promoters include, but are not limited to steroid-inducible promoters such as the promoters for the genes encoding the glucocorticoid or estrogen receptors (inducible by treatment with the corresponding hormone), metallothionine promoter (inducible by treatment with various heavy metals), MX-1 promoter (inducible by interferon), etc. The invention therefore provides lentiviral transfer plasmids as described above comprising a tissue-specific promoter and methods of using the transfer plasmids and lentiviral particles derived therefrom to achieve cell type or tissue specific expression.

Another approach to achieving conditional expression involves use of binary transgenic systems, in which gene expression is controlled by the interaction of two components: a “target” transgene and an “effector” transgene, whose product acts on the target transgene. See, e.g., Lewandoski, M., Nature Reviews Genetics 2, 743-755 (2001) and articles referenced therein, all of which are incorporated herein by reference, reviewing methods for achieving conditional expression in mice. In general, binary transgenic systems fall into two categories. In the first type of system, the effector transactivates transcription of the target transgene. For example, in the tetracycline-dependent regulatory systems (Gossen, M. & Bujard, H, Proc. Natl Acad. Sci. USA 89, 5547-5551 (1992), the effector is a fusion of sequences that encode the VP 16 transactivation domain and the Escherichia coli tetracycline repressor (TetR) protein, which specifically binds both tetracycline and the 1 9-bp operator sequences (tetO) of the tet operon in the target transgene, resulting in its transcription. In the original system, the tetracycline-controlled transactivator (tTA) cannot bind DNA when the inducer is present, while in a modified version, the ‘reverse tTA’ (rtTA) binds DNA only when the inducer is present (‘tet-on’) (Gossen, M. et al., Science 268, 1766-1769 (1995)). The current inducer of choice is doxycycline (Dox). The invention therefore provides lentiviral transfer plasmids as described above comprising a tetracycline-controlled transactivator or reverse tetracycline-controlled transactivator, lentiviral transfer plasmids comprising operator sequences of the tet operon to which the tetracycline-controlled transactivator or reverse tetracycline-controlled transactivator specifically bind, and methods of using the transfer plasmids and lentiviral particles derived therefrom to achieve conditional expression, including the generation of transgenic animals in which conditional expression is achieved.

In the second type of system, the effector is a site-specific DNA recombinase that rearranges the target gene, thereby activating or silencing it. These systems are described below. In order to achieve conditional expression in cells or tissues having a particular physiological, biological, or disease state, a promoter that is selectively active in cells or tissue having that particular physiological, biological, or disease state may be used.

As described further below, one application for the lentiviral transfer plasmids and lentiviral expression systems of the invention is to direct transcription of RNAs that hybridize or self-hybridize to form siRNAs or shRNAs in cells, e.g., mammalian cells. For these purposes in certain embodiments of the invention it is preferred to use a PolIII promoter such as the U6 or H1 promoter. Therefore, the invention provides lentiviral transfer plasmids and lentiviral particles optimized for siRNA, i.e., lentiviral transfer plasmids and lentiviral particles comprising a PolIII promoter, e.g., the U6 or H1 promoter. According to certain embodiments of the invention the PolIII promoter is inducible. It is noted that Pol II promoters can also be used to achieve intracellular expression of siRNA or shRNA (Xia, H., et al., Nat. Biotech., 20: 1006-1010, 2002), and the lentiviral vectors described herein may be used in this manner.

Transfer Plasmid Size.

As described in further detail in Example 1, by removing certain dispensable sequences the inventors have created lentiviral transfer plasmids having reduced size relative to previously known lentiviral transfer plasmids, which results in a number of advantages. First, the reduced size of the transfer constructs adds to their ease of manipulability. Second, the reduced size adds to their flexibility. As is known in the art, there is a limit to the size of retroviral genomes that can be efficiently packaged. Generally it is preferable to limit the size of the transcript for packaging (distance between 5′ and 3′ UTRs) to less than approximately 8-10 kB. Thus removal of the dispensable sequences allows the insertion of larger heterologous sequence(s) without compromising the ability of the resulting genomic transcript to be packaged during the production of lentiviral particles. As used herein in reference to retroviral and lentiviral vectors, a “genome” or “genomic transcript” generally refers to a transcript that contains sufficient packaging signals to allow packaging. It does not imply that the transcript need contain all or even most of the genetic information found in a wild type virus. In general, the sequence of a genomic transcript will depend on the location of the promoter upstream of the packaging sequence and the location of the polyadenylation site downstream of the packaging sequence.

The invention provides a lentiviral transfer plasmid having a length less than 10 kilobases (kB). The invention provides a lentiviral transfer plasmid having a length less than 9 kB. The invention provides a lentiviral transfer plasmid having a length less than 8 kB). The invention provides a lentiviral transfer plasmid having a length less than 7 kB). The invention provides a lentiviral transfer plasmid having a length less than 6.5 kB). The invention provides a lentiviral transfer plasmid having a length of approximately 6 kB). Generally, unless otherwise evident from the context, the term “approximately” means that the value may deviate by 10% or less from the numeral given, and the ranges listed are assumed to include both endpoints. The invention further provides collections of lentiviral plasmids having a length less than 10 kB, a length less than 9 kB, a length less than 8 kB, or a length less than 7 kB.

In particular, the invention provides a lentiviral transfer plasmid having a length less than 8 kB and comprising one or more heterologous nucleic acid sequences. According to certain embodiments of the invention the heterologous nucleic acid sequence is a promoter or promoter/enhancer such as the CMV promoter, the CMV promoter/enhancer, or the Ubiquitin C promoter. According to certain embodiments of the invention the promoter is the U6 or H1 promoter. According to certain embodiments of the invention the heterologous nucleic acid sequence is a reporter gene, e.g., a gene encoding EGFP or dsRed2. The invention particularly provides a lentiviral transfer plasmid having a length of approximately 6.0 kB comprising at least one MCS, two LoxP sites, an HIV FLAP element, and a WRE.

Transfer Plasmid Sequence Information.

The inventors have recognized that prior art lentiviral vector systems suffered from a dearth of sequence information. As will be readily appreciated by one of ordinary skill in the art, regardless of the particular nature of a transfer plasmid, it is desirable to have complete and accurate sequence information. Such information makes it possible, for example, to readily determine the identity of all restriction sites, to design primers for amplification of particular plasmid sequences or for other purposes such as the introduction of mutations, etc. In addition, the availability of complete sequence information makes it possible to identify determinants of plasmid function, e.g., by engineering mutations at specific sites and observing the effect on, for example, packaging, integration, transcription, etc. Accordingly, the invention provides a fully sequenced lentiviral transfer plasmid, wherein the sequence is deposited in a publicly accessible database. By “fully sequenced” is meant that the complete nucleotide sequence of the plasmid is known. By “publicly accessible database” is meant Genbank, or any other database that can be accessed by the public without requiring a fee. In particular, the invention provides a fully sequenced lentiviral transfer plasmid comprising the sequence set forth in any of the following SEQ ID NOS: SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. In addition, the invention provides a collection of lentiviral transfer plasmids including at least two of the plasmids having SEQ ID NOS: SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. In addition, the invention provides a lentiviral transfer plasmid having a sequence that differs by not more than 100 nucleotides from the sequence set forth in SEQ ID NOS: SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The invention further provides a lentiviral transfer plasmid having a sequence that differs by not more than X nucleotides from the sequence set forth in SEQ ID NOS: SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, where X represents any number between 1 and 99, inclusive. By “a sequence that differs by not more than X nucleotides (where X is any number) from the sequence of SEQ ID NO: Y” is meant any sequence that can be obtained from SEQ ID NO: Y by either inserting, deleting, and/or altering less than X nucleotides of SEQ ID NO: Y.

Recombination Sites for Site-Specific Recombinase.

According to certain embodiments of the invention the transfer plasmid includes at least one (typically two) site(s) for recombination mediated by a site-specific recombinase. Site-specific recombinases catalyze the introduction or excision of DNA fragments from a longer DNA molecule. These enzymes recognize a relatively short, unique nucleic acid sequence, which serves for both recognition and recombination. Typically the recombination site is composed of short inverted repeats (6, 7 or 8 base pairs in length) and the length of the DNA-binding element is typiclly 11-13 bp in length.

In general, the transfer plasmids may contain one or more recombination sites for any of a wide variety of site-specific recombinases. As mentioned above, it is to be understood that the target site for a site-specific recombinase is in addition to any site(s) required for integration of the lentiviral genome. According to various embodiments of the invention the transfer plasmid includes one or more sites for a recombinase enzyme selected from the group consisting of Cre, XerD, HP1 and Flp. These enzymes and their recombination sites are well known in the art. See, for example, Sauer, B. & Henderson, N., Nucleic Acids Res. 17, 147-161 (1989), Gorman, C. and Bullock, C., Curr. Op. Biotechnol., 11(5): 455-460, 2000, O'Gorman, S., Fox, D. T. & Wahl, G. M., Science 251, 1351-1355 (1991) and Kolb, A., Cloning Stem Cells, 4(1):65-80, 2002, and U.S. Pat. No. 4,959,317. See also Kuhn, R., and Torres, R M, Methods Mol Biol 2002;180:175-204.

These recombinases catalyse a conservative DNA recombination event between two 34-bp recognition sites (loxP and FRT, respectively). Placing a heterologous nucleic acid sequence operably linked to a promoter element between two loxP sites (in which case the sequence is “floxed”) allows for controlled expression of the heterologous sequence following transfer into a cell. By inducing expression of Cre within the cell, the heterologous nucleic acid sequence is excised, thus preventing further transcription and effectively eliminating expression of the sequence. This system has a number of applications including Cre-mediated gene activation (in which either heterologous or endogenous genes may be activated, e.g., by removal of an inhibitory element or a polyadenylation site), creation of transgenic animals exhibiting temporal control of Cre expression, cell-lineage analysis in transgenic animals, and generation of tissue-specific knockouts or knockdowns in transgenic animals.

According to certain embodiments of the invention the transfer plasmid includes two loxP sites. Furthermore, in preferred embodiments of the invention the transfer plasmid includes a cloning site, e.g., a unique restriction site, between the two loxP sites, which allows the convenient insertion of a heterologous nucleic acid sequence. According to certain embodiments of the invention the transfer plasmid includes a MCS between the two loxP sites. According to certain embodiments of the invention the two loxP sites are located between an HIV FLAP element and a WRE. According to certain embodiments of the invention the plasmid contains a unique restriction site between the 3′ loxP site and the WRE.

As described above, positioning the heterologous nucleic acid sequence between loxP sites allows for controlled expression of the heterologous sequence following transfer into a cell. By inducing Cre expression within the cell, the heterologous nucleic acid sequence is excised, thus preventing further transcription and effectively eliminating expression of the sequence. Cre expression may be induced in any of a variety of ways. For example, Cre may be present in the cells under control of an inducible promoter, and Cre expression may be induced by activating the promoter. Alternately, Cre expression may be induced by introducing an expression vector that directs expression of Cre into the cell. Any suitable expression vector can be used, including, but not limited to, viral vectors such as adenoviral vectors. (The phrase “inducing Cre expression” as used herein refers to any process that results in an increased level of Cre within a cell.)

The invention thus provides a method for achieving controlled expression of a heterologous nucleic acid sequence comprising steps of inserting the heterologous nucleic acid sequence into a transfer plasmid of the invention between sites for a recombinase, thereby producing a modified transfer plasmid; introducing the modified transfer plasmid or a portion thereof including at least the sites for the recombinase and the region between the sites into a cell and; subsequently inducing expression of the recombinase within the cell. According to certain embodiments of the invention the cell is a mammalian cell. According to certain embodiments of the invention the recombinase is Cre and the sites for the recombinase are loxP sites. In accordance with the invention the transfer plasmid may be introduced into the cell using standard techniques such as transfection. Alternately, the transfer plasmid may be used to generate a lentiviral particle that includes a lentiviral genome comprising the recombinase sites and the region between them. As described elsewhere herein, the genome integrates into the cell's DNA and directs expression of the heterologous nucleic acid sequence. The cell may be used for any of a variety of purposes as described in more detail below.

The lentiviral transfer plasmids comprising two loxP sites are useful in any applications for which standard vectors comprising two loxP sites can be used. For example, selectable markers may be placed between the loxP sites. This allows for sequential and repeated targeting of multiple genes to a single cell (or its progeny). After introduction of a transfer plasmid comprising a floxed selectable marker into a cell, stable transfectants may be selected. After isolation of a stable transfectant, the marker can be excised by induction of Cre. The marker may then be used to target a second gene to the cell or its progeny. Lentiviral particles comprising a lentiviral genome derived from the transfer plasmids may be used in the same manner.

As another example, standard gene-targeting techniques may be used to produce a mouse in which an essential region of a gene of interest is floxed, so that tissue-specific Cre expression results in the inactivation of this allele. The transfer plasmids may be introduced into cells (e.g., ES cells) using pronuclear injection. Alternately, the cells may be injected or infected with lentiviral particles comprising a lentiviral genome derived from the transfer plasmid. Tissue-specific Cre expression may be achieved by crossing a mouse line with a conditional allele (i.e., a floxed nucleic acid sequence) to an effector mouse line that expresses cre in a tissue-specific manner, so that progeny are produced in which the conditional allele is inactivated only in those tissues or cells that express Cre. Suitable transgenic lines are known in the art and may be found, for example, in the Cre Transgenic Database at the Web site having URL www.mshri.on.ca/nagy/Cre-pub.htrnl.

Internal Ribosome Entry Sequence (IRES).

The transfer plasmids may also include an IRES. IRES elements function as initiators of the efficient translation of reading frames. An IRES allows ribosomes to start the translation process anew with whatever is immediately downstream and regardless of whatever was upstream. In particular, an IRES allows for the translation of two different genes on a single transcript. For example, an IRES allows the expression of a marker such as EGFP off the same transcript as a transgene, which has a number of advantages: (1) The transgene is native and does not have any fused open reading frames that might affect function; (2) Since the EGFP is from the same transcript its levels should be an accurate representation of the levels of the upstream transgene. IRES elements are known in the art and are further described in Kim, et al., Molecular and Cellular Biology 12(8):3636-3643 (August 1992) and McBratney, et al., Current Opinion in Cell Biology 5:961-965 (1993).

Any of a wide variety of sequences of viral, cellular, or synthetic origin which mediate internal binding of the ribosomes can be used as an IRES. Examples include those IRES elements from poliovirus Type I, the 5′UTR of encephalomyocarditis virus (EMV), of Thelier's murine encephalomyelitis virus (TMEV) of foot and mouth disease virus (FMDV) of bovine enterovirus (BEV), of coxsackie B virus (CBV), or of human rhinovirus (HRV), or the human immunoglobulin heavy chain binding protein (BIP) 5′UTR, the Drosophila antennapediae 5′UTR or the Drosophila ultrabithorax 5′UTR, or genetic hybrids or fragments from the above-listed sequences.

Transfer Plasmids Incorporating Heterologous Nucleic Acids.

The invention provides new lentiviral transfer constructs incorporating a variety of heterologous nucleic acids (also referred to as heterologous sequences or heterologous nucleic acid segments), preferably operably linked to a promoter or promoter/enhancer element. These sequences may be inserted at any available site within the transfer plasmid including, but not limited to, at a restriction site within a MCS. In general, the inserted nucleotide sequence may be any nucleotide sequence and may be a naturally occurring sequence or variant thereof or an artificial sequence. Heterologous gene sequences of the present invention may comprise one or more gene sequences that already possess one or more regulatory elements such as promoters, initiation sequences, processing sequences, etc. Alternatively, such regulatory elements may be present within the transfer plasmid prior to insertion of the heterologous sequence.

According to certain embodiments of the invention the inserted heterologous sequence is a reporter gene sequence. A reporter gene sequence, as used herein, is any gene sequence which, when expressed, results in the production of a protein whose presence or activity can be monitored. Suitable reporter gene sequences include, but are not limited to, sequences encoding chemiluminescent or fluorescent proteins such as green fluorescent protein (GFP) and variants thereof such as enhanced green fluorescent protein (EGFP); cyan fluorescent protein; yellow fluorescent protein; blue fluorescent protein; dsRed or dsRed2, luciferase, aequorin, etc. Many of these markers and their uses are reviewed in van Roessel, P. and Brand, A., Nature Cell Biology, 4(1), E15-20, 2002, and references therein, all of which are incorporated herein by reference. Additional examples of suitable reporter genes include the gene for galactokinase, beta-galactosidase, chloramphenicol acetyltransferase, beta-lactamase, etc. Alternatively, the reporter gene sequence may be any gene sequence whose expression produces a gene product which affects cell physiology or phenotype. In general, a reporter gene sequence typically encodes a protein that is not normally present within a cell into which the transfer plasmid is to be introduced.

According to certain embodiments of the invention the inserted heterologous sequence is a selectable marker gene sequence, which term is used herein to refer to any gene sequence capable of expressing a protein whose presence permits the selective maintenance and/or propagation of a cell which contains it. Examples of selectable marker genes include gene sequences capable of conferring host resistance to antibiotics (e.g., puromycin, ampicillin, tetracycline, kanamycin, and the like), or of conferring host resistance to amino acid analogues, or of permitting the growth of cells on additional carbon sources or under otherwise impermissible culture conditions. A gene sequence may be both a reporter gene and a selectable marker gene sequence. In general, preferred reporter or selectable marker gene sequences are sufficient to permit the recognition or selection of the plasmid in normal cells.

The heterologous sequence may also comprise the coding sequence of a desired product such as a biologically active protein or polypeptide (e.g., a therapeutically active protein or polypeptide) and/or an immunogenic or antigenic protein or polypeptide. Introduction of the transfer plasmid into a suitable cell thus results in expression of the protein or polypeptide by the cell. Alternatively, the heterologous gene sequence may comprise a nucleic acid segment that provides a template for transcription of an antisense RNA, a ribozyme, or, preferably, one or more strands of a short interfering RNA (siRNA) or a precursor thereof such as a short hairpin RNA (shRNA). As described further below, siRNAs and shRNAs targeted to cellular transcripts inhibit expression of such transcripts. Introduction of the transfer plasmid into a suitable cell thus results in production of the siRNA or shRNA, which inhibits expression of the target transcript.

Three and Four Plasmid Lentiviral Expression Systems.

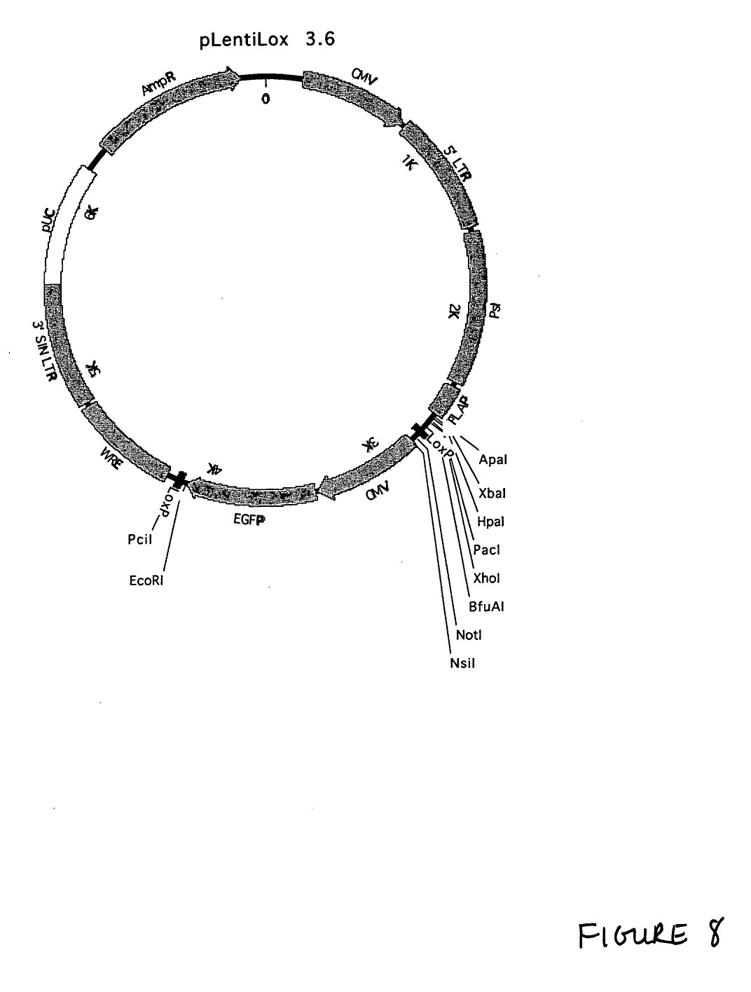

The invention further provides a recombinant lentiviral expression system comprising three plasmids. The first plasmid is constructed to contain mutations that prevent lentivirus-mediated transfer of viral genes. Such mutations may be a deletion of sequences in the viral env gene, thus preventing the generation of replication-competent lentivirus, or may be deletions of certain cis-acting sequence elements at the 3′ end of the genome required for viral reverse transcription and integration. Thus even if viral genes from this construct are packaged into viral particles, they will not be replicated and replication-competent wild-type viruses will not be produced. The first plasmid (packaging plasmid) comprises a nucleic acid sequence of at least part of a lentiviral genome, wherein the vector (i) contains at least one defect in at least one gene encoding a lentiviral structural protein, and (ii) lacks a functional packaging signal. The second plasmid (Env-coding plasmid) comprises a nucleic acid sequence of a virus, wherein the vector (i) expresses a viral envelope protein, and (ii) lacks a functional packaging signal. The third plasmid may be any of the inventive transfer plasmids described above. The first and second plasmids are further described below, and schematic diagrams of relevant portions of representative first and second plasmids (packaging and Env-coding) are presented in FIG. 10A, which is taken from reference 21. The third plasmid (not shown) is a transfer plasmid.

Packaging Plasmid.

In certain embodiments of the invention the first vector is a gag/pol expression vector, i.e., a plasmid capable of directing expression of functional forms of a retroviral gag gene product and a retroviral Pol gene product. These proteins are necessary for assembly and release of viral particles from cells. The first plasmid may also express sequences encoding various accessory lentiviral proteins including, but not limited to, Vif, Vpr, Vpu, Tat, Rev, and Nef. In particular, the first plasmid may express a sequence encoding Rev. In general, the gag and pol sequences may be derived from any retrovirus, and the accessory sequences may be derived from any lentivirus. According to certain embodiments of the invention the gag and pol sequences and any accessory sequences are derived from HIV-1. It is noted that the gag, pol, and accessory protein sequences need not be identical to wild type versions but instead may contain mutations, deletions, etc., that do not significantly impair the ability of the protein to perform its function in the viral life cycle.

The first plasmid is preferably constructed to contain mutations that exclude retroviral-mediated transfer of viral genes. Such mutations may be a deletion or mutation of sequences in the viral env gene, thus excluding the possibility of generating replication-competent lentivirus. Alternatively, or in addition to, deletion or mutation of env, according to certain embodiments of the invention the plasmid sequence may contain deletions of certain cis-acting sequence elements at the 3′ end of the genome required for viral reverse transcription and integration. Accordingly, even if viral genes from this construct are packaged into viral particles, they will not be replicated and replication-competent wild-type viruses will not be generated. Any of a wide variety of packaging plasmids may be used in the three plasmid lentiviral expression system of the invention including, but not limited to, those described in references 21, 24, 29, and 40.

Env-Coding Plasmid. This plasmid directs expression of a viral envelope protein and, therefore, comprises a nucleic acid sequence encoding a viral envelope protein under the control of a suitable promoter. The promoter can be any promoter capable of directing transcription in cells into which the plasmid is to be introduced. One of ordinary skill in the art will readily be able to select an appropriate promoter among, for example, the promoters mentioned above. For example, according to certain embodiments of the invention a CMV promoter is used. The Env-coding plasmid preferably contains any additional sequences needed for efficient transcription, processing, etc., of the env transcript including, but not limited to, a polyadenylation signal such as any of those mentioned above.

The host range of cells that the viral vectors of the present invention can infect may be altered (e.g., broadened) by utilizing an envelope gene from a different virus. Thus is possible to alter or increase the host range of the vectors of the present invention by taking advantage of the ability of the envelope proteins of certain viruses to participate in the encapsidation of other viruses. In a preferred embodiment of the present invention, the G-protein of vesicular-stomatitis virus (VSV-G; see, e.g., Rose and Gillione, J. Virol. 39, 519-528 (1981); Rose and Bergmann, Cell 30, 753-762 (1982)), or a fragment or derivative thereof, is the envelope protein expressed by the second plasmid. VSV-G efficiently forms pseudotyped virions with genome and matrix components of other viruses. As used herein, the term “pseudotype” refers to a viral particle that contains nucleic acid of one virus but the envelope protein of another virus. In general, VSV-G pseudotyped viruses have a very broad host range, and may be pelleted to titers of high concentration by ultracentrifugation (e.g., according to the method of J. C. Burns, et al., Proc. Natl. Acad. Sci. USA 90, 8033-8037 (1993)), while still retaining high levels of infectivity.

Additional envelope proteins that may be used in accordance with the present invention include, but are not limited to, ecotropic or amphotropic MLV envelopes, 10A1 envelope, truncated forms of the HIV env, GALV, BAEV, SIV, FeLV-B, RD114, SSAV, Ebola, Sendai, FPV (Fowl plague virus), and influenza virus envelopes. Similarly, genes encoding envelopes from RNA viruses (e.g. RNA virus families of Picomaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxviridae, and Iridoviridae) may be utilized. Representative examples include FIV, FeLV, RSV, VEE, HFVW, WDSV, SFV, Rabies, ALV, BIV, BLV, EBV, CAEV, HTLV, SNV, ChTLV, STLV, MPMV, SMRV, RAV, FuSV, MH2, AEV, AMV, CT10, EIAV. In addition to the above, hybrid envelopes (e.g. envelope comprising regions of more than one of the above), may be employed. According to certain embodiments of the invention the envelope recognizes a unique cellular receptor (e.g., a receptor found only on a specific cell type or in a specific species), while according to certain other embodiments of the invention the envelope recognizes multiple different receptors. According to certain embodiments of the invention the second plasmid encodes a cell or tissue specific targeting envelope. Cell or tissue specific targeting may be achieved, for example, by incorporating particular sequences within the envelope sequence (e.g., sequences encoding ligands for cell or tissue-specific receptors, antibody sequences, etc.). Thus any of a wide variety of Env-coding plasmids may be used in the three plasmid lentiviral expression system of the invention including, but not limited to, those described in references 21, 24, 29, and 40.

Variations on the Three Plasmid System.

The invention further provides a four plasmid lentiviral expression system comprising a three plasmid lentiviral expression system as described herein and a fourth plasmid comprising a nucleic acid sequence encoding the Rev protein (in which case the rev gene is generally not included in the other plasmids. As mentioned above, the presence of Rev increases the level of transcription during production of lentiviral particles. It will be appreciated that a variety of alternative three or four plasmid systems may be employed while maintaining the feature that no sequence of recombination event(s) between only two of the three or four plasmids is sufficient to generate replication-competent virus. For example, either Gag or Pol or any of the accessory proteins may be encoded by the plasmid referred to as the Env-coding plasmid. Alternately, Gag, Pol, or any of the accessory proteins may be encoded by the transfer plasmid. In addition, sequences encoding Rev may be provided on the same plasmid that encodes Gag, Pol, or Env. According to certain embodiments of the invention sequences encoding a functional Tat protein are absent from the plasmids, and sequences encoding Rev are provided on a separate plasmid rather than on the same plasmid as sequences encoding other viral genes, as described in reference 40. Schematic diagrams of relevant portions of representative first and second plasmids (packaging and Env-coding) and fourth plasmid encoding Rev are presented in FIG. 10B, which is taken from reference 40. The third plasmid (not shown) is a transfer plasmid.

Applications of the Lentiviral Transfer Plasmids and Expression Systems.

The lentiviral transfer plasmids and lentiviral expression systems of the invention have a wide variety of uses, some of which have been described above. As will be evident, the transfer plasmids may be used for any application in which a conventional expression plasmid is employed. As described in Examples 3 through 6, the transfer plasmids of the invention are able to drive expression of heterologous genes (e.g., EGFP) when transfected into cells and are also able to drive synthesis of shRNA when transfected into cells.

The presence of one or more MCSs means that the plasmids may conveniently be used for insertion and subsequent expression of any heterologous sequence. In particular, the transfer plasmids that include an insertion site such as an MCS between sites for a recombinase such as loxP may be used for easy assembly of a promoter-site-sequence-site cassette, (where “site” indicates a recombination site for a recombinase and “sequence” indicates a heterologous sequence of interest), e.g., a promoter-loxP-sequence-loxP site that can then be moved into another vector. It is noted that the transfer plasmids can be used to direct expression of a heterologous nucleic acid in a variety of eukaryotic cells other than mammalian cells, provided a promoter capable of directing transcription in such cells is employed. Thus references to “mammalian cells” herein should not be understood to exclude non-mammalian cells, as long as an appropriate promoter for transcription in non-mammalian cells is provided.

Introducing Plasmids into Cells.

In general, the plasmids described herein may be introduced into cells via conventional transformation or transfection techniques. As used herein, the terms “transformation” and “transfection” are intended to refer to a variety of art-recognized techniques for introducing foreign nucleic acid (e.g., DNA or RNA) into cells, including calcium phosphate or calcium chloride co-precipitation, DEAE-dextran-mediated transfection, lipofection, injection, or electroporation.

Production of Replication-Defective Lentiviral Particles.

In general, the transfer plasmids and the three-plasmid recombinant lentiviral expression systems of the invention may be used to produce infectious, replication-defective lentiviral particles according to methods known to those skilled in the art. In the case of the recombinant lentiviral expression system of the invention the methods include (i) transfecting a lentivirus-permissive cell with the three-plasmid lentiviral expression system of the present invention; (ii) producing the lentivirus-derived particles in the transfected cell; and (iii) collecting the virus particles from the cell. The step of transfecting the lentivirus-permissive cell can be carried out according to any suitable means known to those skilled in the art. For example, the three-plasmid expression system described herein may be used to generate lentivirus-derived retroviral vector particles by transient transfection. The plasmids may be introduced into cells by any suitable means, including, but not limited to, calcium phosphate or calcium chloride co-precipitation, DEAE-dextran-mediated transfection, lipofection, injection, or electroporation.

The transfer plasmids of the invention may be used to produce infectious, replication-defective lentiviral particles in a similar manner using helper cells that express the necessary viral proteins as known in the art and mentioned above. In general, the transfer plasmids may be used to produce infectious, replication-defective lentiviral particles in conjunction with any system using any combination of plasmids and/or helper cell lines that provides the appropriate combination of required genes: gag, pol, env, and, preferably, rev in cases where transcription occurs from a gag/pol expression cassette containing a Rev-response element (or alternately a system that supplies the various proteins encoded by these genes).

Infectious virus particles may be collected using conventional techniques. For example, the infectious particles may be collected by cell lysis, or collection of the supernatant of the cell culture, as is known in the art. Optionally, the collected virus particles may be purified if desired. Suitable purification techniques are well known to those skilled in the art. Methods for titering virus particles are also well known in the art. Further details are provided in the Examples.

Producer Cell Lines.

As will be evident, when a host cell permissive for production of lentiviral particles is transfected with the plasmids of the three-plasmid system, the cell becomes a producer cell, i.e., a cell that produces infectious lentiviral particles. Similarly, when a helper cell that produces the necessary viral proteins is transfected with a transfer plasmid of the invention, the cell becomes a producer cell. The invention therefore provides producer cells and corresponding producer cell lines and methods for the production of such cells and cell lines. In particular, the invention provides a method of creating a producer cell line comprising introducing a transfer plasmid of the invention into a host cell; and introducing a packaging plasmid and an envelope plasmid into the host cell. The invention provides another method of creating a producer cell line comprising introducing a transfer plasmid of the invention into a helper cell that produces viral proteins necessary for encapsidation of a lentiviral genome and subsequent infectivity of a lentiviral particle resulting from encapsidation.

The inclusion of appropriate genetic elements from various papovaviruses allows plasmids to be maintained as episomes within mammalian cells. Such plasmids are faithfully distributed to daughter cells. In particular, viral elements of various polyomaviruses and papillomaviruses such as BK virus (BKV), bovine papilloma virus 1 (BPV-1) and Epstein-Barr virus (EBV), among others, are useful in this regard. The invention therefore provides lentiviral transfer plasmids comprising a viral element sufficient for stable maintenance of the transfer plasmid as an episome within mammalian cells. Appropriate genetic elements and their use are described, for example, in Van Craenenbroeck, et al., Eur. J. Biochem. 267, 5665-5678 (2000) and references therein, all of which are incorporated herein by reference.

The invention further provides cell lines comprising the transfer plasmids described above, i.e., cell lines in which the transfer plasmids are stably maintained as episomes. In particular, the invention provides producer cell lines (cell lines that produce the proteins needed for production of infectious lentiviral particles) in which the transfer plasmids are stably maintained as episomes. According to certain embodiments of the invention these cell lines constitutively produce lentiviral particles.

According to other embodiments of the invention one or more of the necessary viral proteins is under the control of an inducible promoter. Thus the invention provides helper cell lines in which the transfer plasmids are stably expressed as episomes, wherein at least one viral protein expressed by the cell line is under control of an inducible promoter. This allows the cells to be expanded under conditions that are not permissive for viral production. Once the cells have reached a desired density (e.g., confluence), or a desired cell number, etc., the protein whose expression is under control of the inducible promoter can be induced, allowing production of viral particles to begin. This system offers a number of advantages. In particular, since every cell has the required components, titer is increased. In addition, it avoids the necessity of performing a transfection each time a particular virus is desired. Any of a variety of inducible promoters known in the art may be used. One of ordinary skill in the art will readily be able to select an appropriate inducible promoter and apply appropriate techniques to induce expression therefrom.

The invention thus provides a method of producing lentiviral particles comprising introducing a lentiviral transfer plasmid of the invention, which lentiviral transfer plasmid comprises a genetic element (e.g., a viral element) sufficient for stable maintenance of the transfer plasmid as an episome in mammalian cells, into a helper cell that produces proteins needed for production of infectious lentiviral particles and; culturing the cell for a period sufficient to allow production of lentiviral particles. The invention further provides a method of producing lentiviral particles comprising introducing a lentiviral transfer plasmid of the invention, which lentiviral transfer plasmid comprises a genetic element sufficient for stable maintenance of the transfer plasmid as an episome in mammalian cells, into a helper cell that expresses a protein required for production of lentiviral particles, wherein expression of the protein is under control of an inducible promoter; inducing expression of the protein required for production of lentiviral particles; and culturing the cell for a period sufficient to allow production of lentiviral particles.

Transgenic and Knockout Animals.

The transfer plasmids may be used to generate stable transgenic or knockout animals, wherein the transgene is a heterologous nucleic acid contained in the transfer plasmid. Transgenic animals may be generated through standard (non-viral) means such as pronuclear injection of the transfer plasmid. In addition, the lentiviral particles may be used to create transgenic animals wherein the transgene is a heterologous nucleic acid contained in the lentiviral particle. For example, lentiviral particles of the invention may be injected into the perivitelline space of single-cell embryos, which may then be implanted and carried to term. Alternately, the zona pellucida may be removed and the denuded embryo incubated with lentiviral suspension prior to implantation as described in reference 24. This approach offers a more efficient method of creating a variety of transgenic animals, e.g., birds, rats, and other mammals. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. Transgenic animals typically carry a gene which has been introduced into the germline of the animal, or an ancestor of the animal, at an early (usually one-cell) developmental stage. In general, a transgene is heterologous DNA, which preferably is integrated into or occurs in the genome of the cells of a transgenic animal. Integration of the transgene may lead to a deletion of endogenous chromosomal DNA, e.g., by homologous recombination, such that the function of an expression product of the DNA is impaired or eliminated. In this case the resulting animal is referred to as a knockdown or knockout animal. Note that transgene sequences may include endogenous sequences but typically also include additional sequences that do not naturally occur in the animal.

As described in Example 7, the inventors have generated transgenic mice using a lentiviral particle comprising a heterologous nucleic acid encoding the fluorescent protein GFP, which serves as a transgene. The lentiviral particles were able to induce expression of GFP within embryonic stem cells (ES cells), and these ES cells gave rise to transgenic animals whose cells expressed GFP. These results demonstrate that heterologous nucleic acids contained in the lentiviral particles of the invention are not subject to developmental silencing.

Constitutive, Conditional, Reversible, and Tissue-Specific Expression.

The transfer plasmids and lentiviral particles of the invention may be used to achieve constitutive, conditional, reversible, or tissue-specific expression in cells, tissues, or organisms, including transgenic animals. The invention provides a method of reversibly expressing a transcript in a cell comprising: (i) delivering a lentiviral vector to the cell, wherein the lentiviral vector comprises a heterologous nucleic acid, and wherein the heterologous nucleic acid is located between sites for a site-specific recombinase; and (ii) inducing expression of the site-specific recombinase within the cell, thereby preventing synthesis of synthesis of the transcript within those cells. According to certain embodiments of the invention the cell is a mammalian cell. According to certain embodiments of the invention the step of inducing the site-specific recombinase comprises introducing a vector encoding the site-specific recombinase into the cell. According to other embodiments of the invention a nucleic acid encoding the site-specific recombinase is operably linked to an inducible promoter, and the inducing step comprises inducing the promoter as described above. As discussed in more detail in Example 8, the inventors have shown that introduction of a lentiviral particle comprising a heterologous nucleic acid encoding the fluorescent protein EGFP between loxP sites into cells results in expression of EGFP within the cells. When the EGFP-expressing cells were subsequently infected with an adenovirus containing a nucleic acid encoding Cre, thereby inducing expression of Cre within the cells, expression of EGFP was eliminated in a significant proportion of the cells. Thus expression of EGFP was reversible.

In addition, the invention provides a variety of methods for achieving conditional and/or tissue-specific expression. For example, the invention provides a method for expressing a transcript in a mammal in a cell type or tissue-specific manner comprising: (i) delivering a lentiviral transfer plasmid or lentiviral particle to cells of the mammal, wherein the lentiviral transfer plasmid or lentiviral particle comprises a heterologous nucleic acid, and wherein the heterologous nucleic acid is located between sites for a site-specific recombinase; and (ii) inducing expression of the site-specific recombinase in a subset of the cells of the mammal, thereby preventing synthesis of the transcript within those cells. According to certain embodiments of the inventive methods the recombinase is Cre. According to certain embodiments of the invention the step of inducing the site-specific recombinase comprises introducing a vector encoding the site-specific recombinase into the cell. According to other embodiments of the invention a nucleic acid encoding the site-specific recombinase is operably linked to an inducible promoter, and the inducing step comprises inducing the promoter as described above. In certain embodiments of the invention the nucleic acid encoding the site-specific recombinase is operably linked to a cell type or tissue-specific promoter, so that synthesis of the recombinase takes place only in cells or tissues in which that promoter is active.

Gene and Transcript Silencing.

As described in more detail below, the invention provides methods of reducing or inhibiting the expression of target genes and/or transcripts (which need not necessarily encode proteins) by exploiting the phenomenon of RNA interference (RNAi). For example, the invention provides a method of inhibiting or reducing the expression of a target transcript in a cell comprising delivering a lentiviral vector (e.g., a lentiviral transfer plasmid or lentiviral particle) to the cell, wherein presence of the lentiviral vector within a cell results in synthesis of one or more RNAs that self-hybridize or hybridize with each other to form a short hairpin RNA or short interfering RNA that is targeted to the target transcript. Such lentiviral expression vectors may be used therapeutically to silence disease-causing genes and/or render cells resistant to infectious organisms. In addition, lentiviral expression vectors may facilitate the creation of animals deficient in immunogenic xenoantigens as sources of organs for organ transplantation. It will be appreciated that in those embodiments of the invention in which the nucleic acid segment that provides a template for synthesis of the one or more RNAs that self-hybridize or hybridize with each other to form an shRNA or siRNA is floxed, inhibition of the target transcript may be reversed by expressing Cre, thereby excising the template for the siRNA or shRNA. Thus the invention allows conditional and tissue-specific expression of target transcripts in cells, tissues, or organisms. RNAi and methods of using the plasmids and expression systems of the invention for achieving RNAi are described below.

RNA Interference

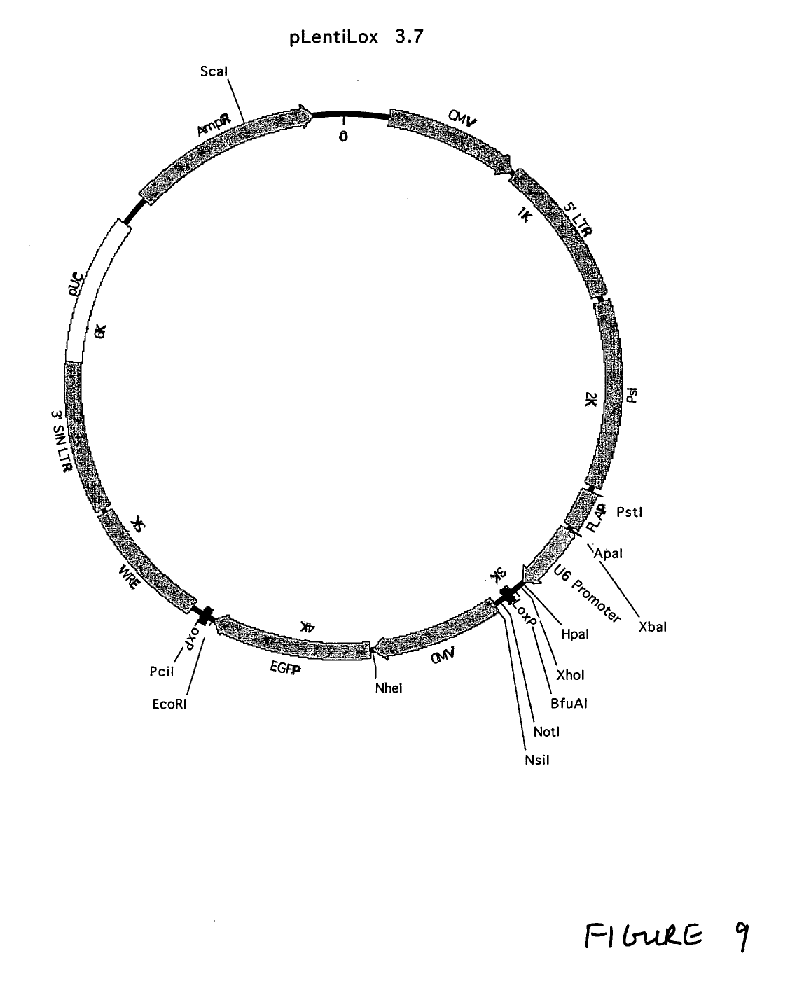

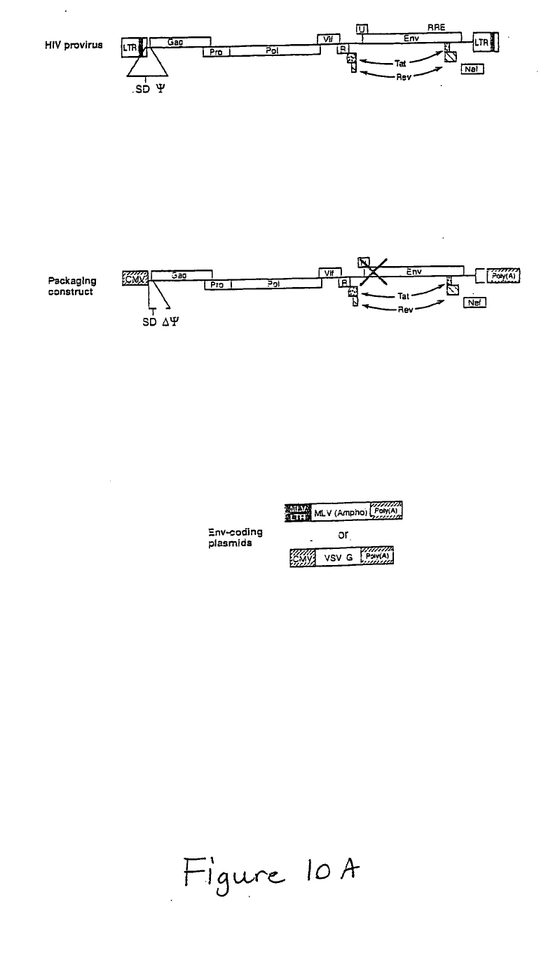

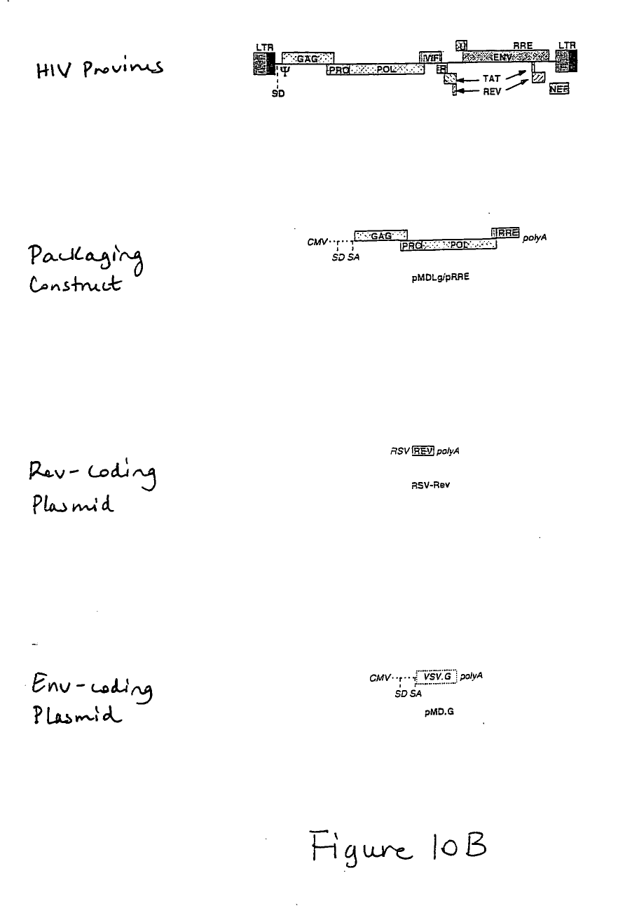

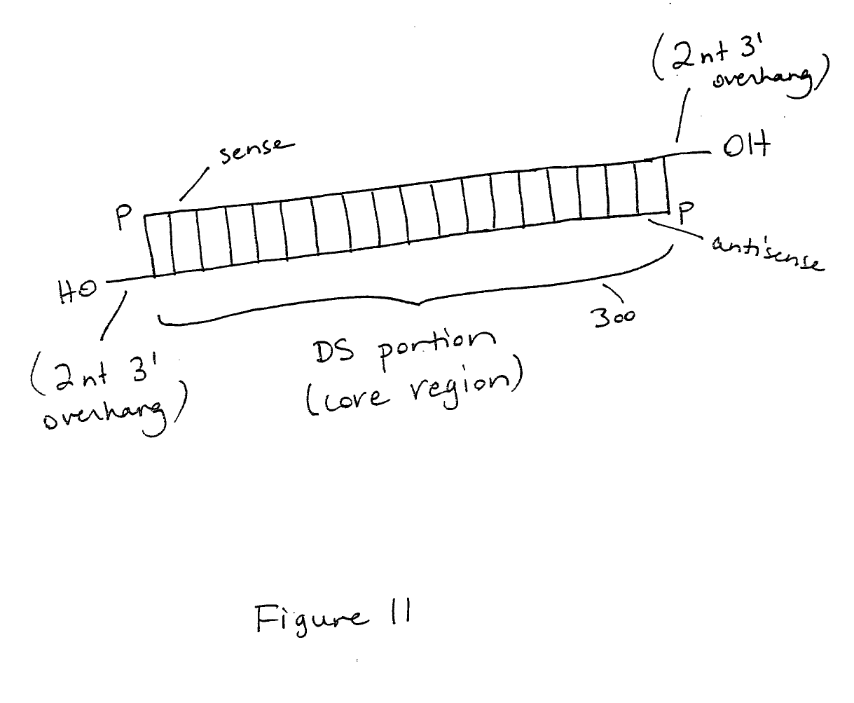

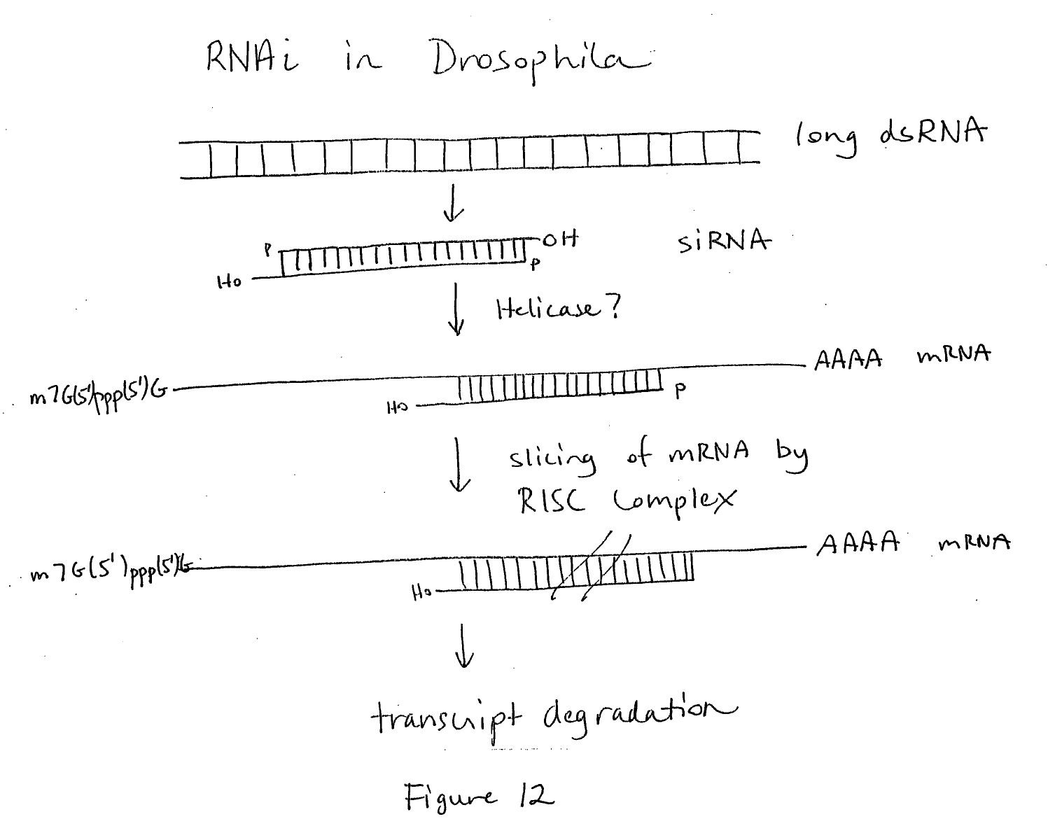

Small inhibitory RNAs were first discovered in studies of the phenomenon of RNA interference (RNAi) in Drosophila, as described, for example, in WO 01/75164, etc. It was found that, in Drosophila, long double-stranded RNAs are processed by an RNase III-like enzyme called DICER (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs (see FIG. 11). These small dsRNAs (siRNAs) act to silence expression of any gene that includes a region complementary to one of the dsRNA strands, presumably because a helicase activity unwinds the 19 bp duplex in the siRNA, allowing an alternative duplex to form between one strand of the siRNA and the target transcript. This new duplex then guides an endonuclease complex, RISC, to the target RNA, which it cleaves (“slices”) at a single location, producing unprotected RNA ends that are promptly degraded by cellular machinery (see FIG. 12).

Homologs of the DICER enzyme are found in diverse species ranging from C. elegans to humans (Sharp, Genes Dev. 15;485, 2001; Zamore, Nat. Struct. Biol. 8:746, 2001), raising the possibility that an RNAi-like mechanism might be able to silence gene expression in a variety of different cell types including mammalian, or even human, cells. However, long dsRNAs (e.g., dsRNAs having a double-stranded region longer than about 30-50 nucleotides) are known to activate the interferon response in mammalian cells. Thus, rather than achieving the specific gene silencing observed with the Drosophila RNAi mechanism, the presence of long dsRNAs into mammalian cells would be expected to lead to interferon-mediated non-specific suppression of translation, potentially resulting in cell death. Long dsRNAs are therefore not thought to be useful for inhibiting expression of particular genes in mammalian cells.

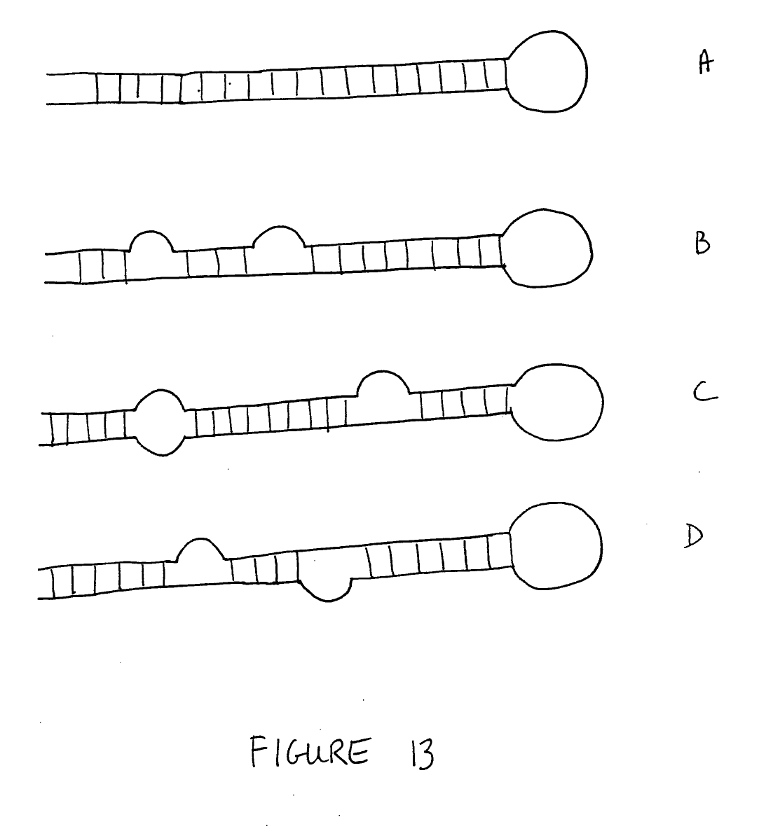

In contrast, siRNAs, when present in mammalian cells, can effectively reduce the expression of target transcripts and genes in a specific manner without activating the anti-viral response (9, 10). Preferred siRNAs typically include a base-paired region approximately 19 nt long, and may further comprise one or more single-stranded regions, typically 3′ overhangs on one or both strands. FIGS. 11 and 13 presents various structures that can be utilized to mediate RNA interference. FIG. 11 shows the siRNA structure found to be active in the Drosophila system and likely represents the species that is active in mammalian cells. This structure consists of two 21 nt strands having a complementary core region of 19 nt and 2 nt 3′ overhangs at each end of the double-stranded region. FIGS. 13A, 13B, 13C, and 13D represent additional structures that may be used to mediate RNA interference. These hairpin (stem-loop) structures may function directly as inhibitory RNAs or may be processed intracellularly to yield an siRNA structure such as that depicted in FIG. 11.

Many different RNA species having structures such as these have been introduced into mammalian cells and have been shown to reduce expression of target transcripts. For example, siRNAs targeted to transcripts encoding the HIV Gag protein or the the HIV-1 cellular receptor CD4 reduced the level of the corresponding mRNAs and proteins Gag in cells infected with HIV (Novina, C., et al., Nat Med, 8(7):681-6, 2002), resulting in inhibition of virus production. Studies such as this, demonstrating siRNA-mediated inhibition of cellular genes as well as genes of infectious organisms, demonstrate the therapeutic potential of RNA interference for a wide variety of conditions. In addition, the ability to selectively reduce or eliminate expression of particular genes has profound implications for the study of gene function.

In general, preferred siRNAs reduce the target transcript level or level of the encoded protein at least about 2 fold, preferably at least about 5 fold, more preferably at least about 10 fold, at least about 25 fold, at least about 50 fold or to an even greater degree relative to the level that would be present in the absence of the inhibitory RNA. In selecting a target sequence for any particular transcript it may be desirable to test a variety of siRNAs in order to identify one with an appropriate efficacy.

In general, an siRNA includes a double-stranded region (the “inhibitory region”), one strand of which is substantially complementary to a portion of the target transcript, so that a precise hybrid can form in vivo between one strand of the siRNA and the target transcript. The portion of the target transcript to which the siRNA strand hybridizes may be referred to as the target or targeted portion or site. In certain preferred embodiments of the invention, the relevant inhibitor region of the siRNA is perfectly complementary with the target transcript; in other embodiments, one or more non-complementary residues are located at or near the ends of the siRNA/template duplex or elsewhere. As will be appreciated by those of ordinary skill in the art, it is generally preferred that mismatches in the central portion of the siRNA/template duplex be avoided (see, for example, Elbashir et al., EMBO J. 20:6877,2001).

Generally any portion of a target transcript may be selected as the target site, to which the antisense strand of the siRNA will be complementary. It may be preferable to select siRNAs that hybridize with a target site that includes exonic sequences in the target transcript or hybridizes exclusively with exonic sequences. Hybridization with intronic sequences is not excluded, but generally appears not to be preferred in mammalian cells. An siRNA that hybridizes with a target site that includes only sequences within a single exon may be selected, or the target site may be created by splicing or other modification of a primary transcript. Any site that is available for hybridization with an siRNA antisense strand, resulting in slicing and degradation of the transcript may be utilized in accordance with the present invention. Nonetheless, those of ordinary skill in the art will appreciate that, in some instances, it may be desirable to select particular regions of target gene transcript as siRNA hybridization targets. For example, it may be desirable to avoid (i) sections of target transcript that may be shared with other transcripts whose degradation is not desired; (ii) sections of target transcript that are identical or homologous to other transcripts whose degradation is not desired. In general, coding regions and regions closer to the 3′ end of the transcript than to the 5′ end are preferred. The 3′ portion of target transcripts may be less likely to exhibit secondary structure that may inhibit or interfere with siRNA activity, e.g., by reducing accessibility.

In general, preferred siRNA sequences have a GC content between 30 and 70% or, preferably, between 40 and 60%. In general, it is preferred to avoid target sequences that contain strings of >2 identical nucleotides (e.g., AAA, GGGG). siRNA sequences may conveniently be identified by scanning the cDNA sequence from 5′ to 3′ until an appropriate 19 nucleotide target is identified. If it is desired to include a 3′ overhang in the antisense strand, the 19 nt sequence should be preceded by nucleotides complementary to the desired 3′ overhang. For example, according to certain embodiments of the invention an siRNA sequence should correspond to: AAN₁₉.

Certain siRNAs hybridize to a target site that includes or consists entirely of 3′ UTR sequences. Such siRNAs may tolerate a larger number of mismatches in the siRNA/template duplex, and particularly may tolerate mismatches within the central region of the duplex. In fact, some mismatches may be desirable as siRNA/template duplex formation in the 3′ UTR may inhibit expression of a protein encoded by the template transcript by a mechanism related to but distinct from classic RNA inhibition. In particular, there is evidence to suggest that siRNAs that bind to the 3′ UTR of a template transcript may reduce translation of the transcript rather than decreasing its stability. Specifically, as shown in FIG. 14, the DICER enzyme that generates siRNAs in the Drosophila system discussed above and also in a variety of organisms, is known to also be able to process a small, temporal RNA (stRNA) substrate into an inhibitory agent that, when bound within the 3′ UTR of a target transcript, blocks translation of the transcript (see Grishok, A., et al., Cell 106, 23-24, 2001; Hutvagner, G., et al., Science, 293, 834-838, 2001; Ketting, R., et al., Genes Dev., 15, 2654-2659). For the purposes of the present invention, any partly or fully double-stranded short RNA as described herein, one strand of which binds to a target transcript and reduces its expression (i.e., reduces the level of the transcript and/or reduces synthesis of the polypeptide encoded by the transcript) is considered to be an siRNA, regardless of whether the RNA acts by triggering degradation, by inhibiting translation, or by other means. In certain preferred embodiments of the invention, reducing expression of the transcript involves degradation of the transcript. In addition any precursor structure (e.g., a short hairpin RNA, as described herein) that may be processed in vivo (i.e., within a cell or organism) to generate such an siRNA is useful in the practice of the present invention.

Use of RNAi in mammalian cells, tissues, and organisms is currently restricted by the limited delivery methods available. siRNAs can be delivered to cells by various means, such as electroporation (11), use of lipofectants (10), or expression of short hairpin RNAs (shRNAs) in cells from a plasmid template (11-16). shRNAs are precursors of siRNAs, and typically comprise dsRNA stretches of at least 19 bp separated by a loop of several non self-complementary nucleotides. shRNAs adopt stem-loop structures, thought to be recognized and processed into siRNAs by the conserved cellular RNAi machinery (17; Ketting, R., et al., Genes Dev., 15, 2654-2659). While the approaches mentioned above have been successful at targeting gene expression in cell culture systems, in general they are not as readily applicable to primary cells, which are difficult to transfect by standard methods such as those mentioned above. Their use to target gene expression in mammalian subjects is also problematic. A further limitation of introducing siRNAs into cells by standard means is that the inhibitory (knockdown) effect is transient, as mammalian cells appear to lack the siRNA amplification mechanisms that confer RNAi potency and longevity in lower organisms (10).

The present invention encompasses the recognition that use of lentiviral expression systems offer a means of overcoming problems associated with delivery of siRNAs into mammalian cells and tissues, including primary mammalian cells and tissues, nondividing cells (including neurons and naive T cells), and cells at early stages of development such as embryonic cells (including embryonic stem cells). The invention further emcompasses the recognition that use of lentiviral vectors offers a means of overcoming problems associated with delivery of siRNAs into mammalian subjects.

The invention provides lentiviral vectors and expression systems capable of directing transcription of RNAs that hybridize to form shRNAs and/or siRNAs in mammalian cells. In particular, the invention provides a lentiviral vector comprising a nucleic acid segment operably linked to a promoter, so that transcription from the promoter results in synthesis of one or more RNAs that self-hybridize or hybridize with each other to form an shRNA or siRNA targeted to a target transcript.

The invention further provides a three-plasmid lentiviral expression system comprising (i) a lentiviral transfer plasmid comprising a nucleic acid segment operably linked to a promoter, so that transcription from the promoter results in synthesis of one or more RNAs that self-hybridize or hybridize with each other to form an shRNA or siRNA targeted to a target transcript; (ii) a packaging plasmid; and (iii) an Env-coding plasmid. In addition, the invention provides an infectious lentiviral particle comprising a nucleic acid segment operably linked to a promoter, so that transcription from the promoter results in synthesis of one or more RNAs that self-hybridize or hybridize with each other to form an shRNA or siRNA targeted to a target transcript. In other words, the nucleic acid segment(s) provides template(s) for synthesis of an RNA that self-hybridizes to form an shRNA or for synthesis of two complementary RNAs that hybridize to form an siRNA.

According to certain embodiments of the invention the lentiviral vector comprises a nucleic acid segment which, when transcribed, produces an RNA that comprises two complementary elements that hybridize to one another to form a stem and a loop. The stem-loop structure is also referred to as a hairpin. FIG. 15A schematically depicts such a nucleic acid segment 10 operably linked to a promoter element 20. Nucleic acid segment 10 comprises complementary elements 30 and 40, separated by element 50. Preferably the nucleic acid includes a transcriptional terminator element 60, e.g., a terminator for RNA polymerase III such as a string of T residues. However, such a terminator element may also be provided within a vector into which the nucleic acid segment is inserted. FIG. 15B schematically depicts an RNA 70 transcribed from nucleic acid segment 10 prior to hybridization. RNA 70 comprises self-complementary elements 80 and 90.

FIG. 15C schematically depicts the RNA following hybridization of the complementary portions, resulting in formation of stem 100 and loop 110. Termination within the terminator sequence results in a 3′ overhang 120, which may comprise one or more U residues. Preferably, the stem is approximately 19 bp long, the loop is about 1-20, more preferably about 4-12, and most preferably about 6-10 nt long and/or the overhang is about 1-20, and more preferably about 2-6 nt long. In certain preferred embodiments of the invention the overhang is 2 nt long. In certain embodiments of the invention the stem is minimally 19 nucleotides in length and may be up to approximately 29 nucleotides in length. One of ordinary skill in the art will appreciate that loops of 4 nucleotides or greater are less likely to be subject to steric constraints than are shorter loops and therefore may be preferred.

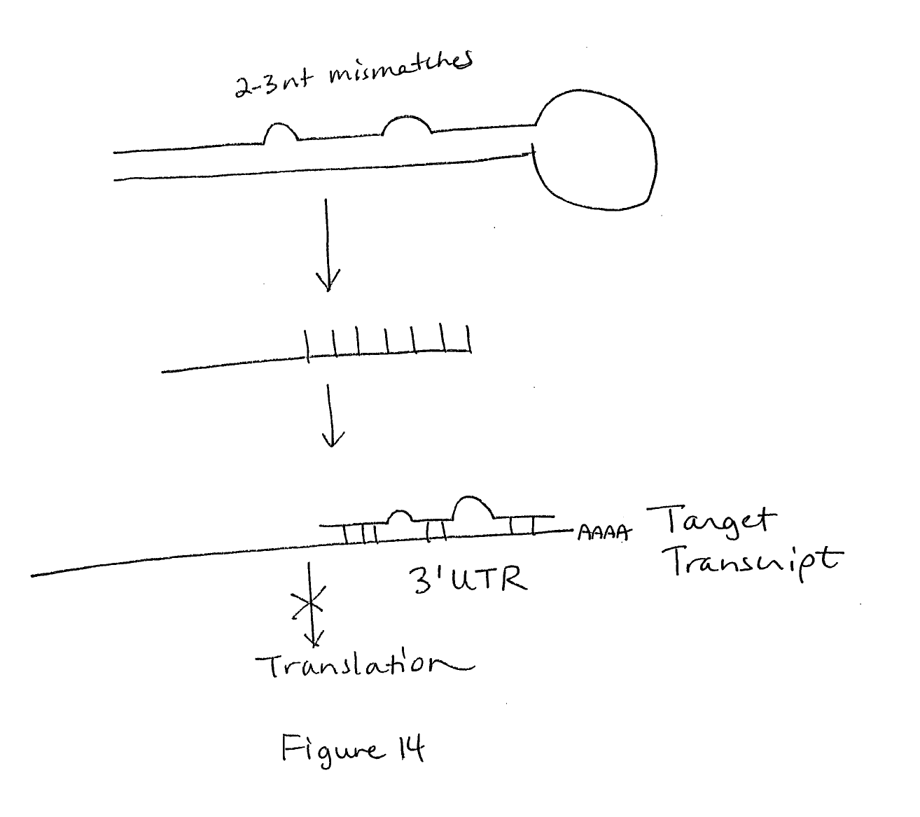

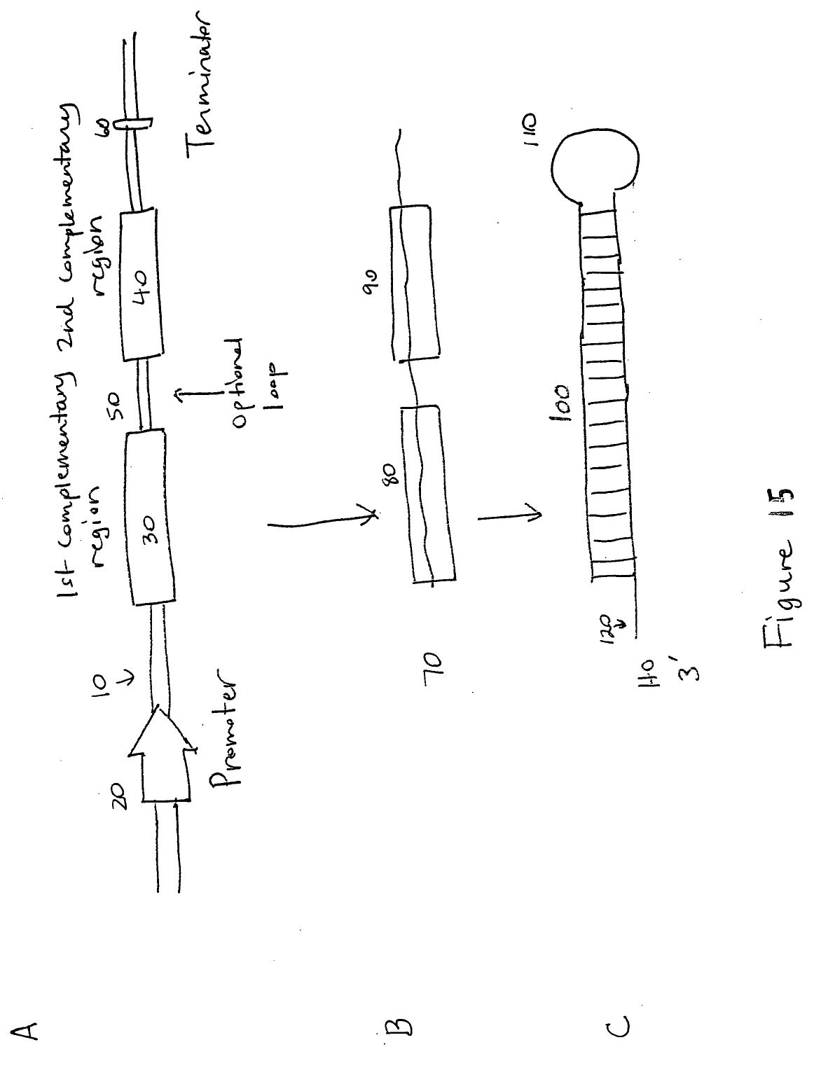

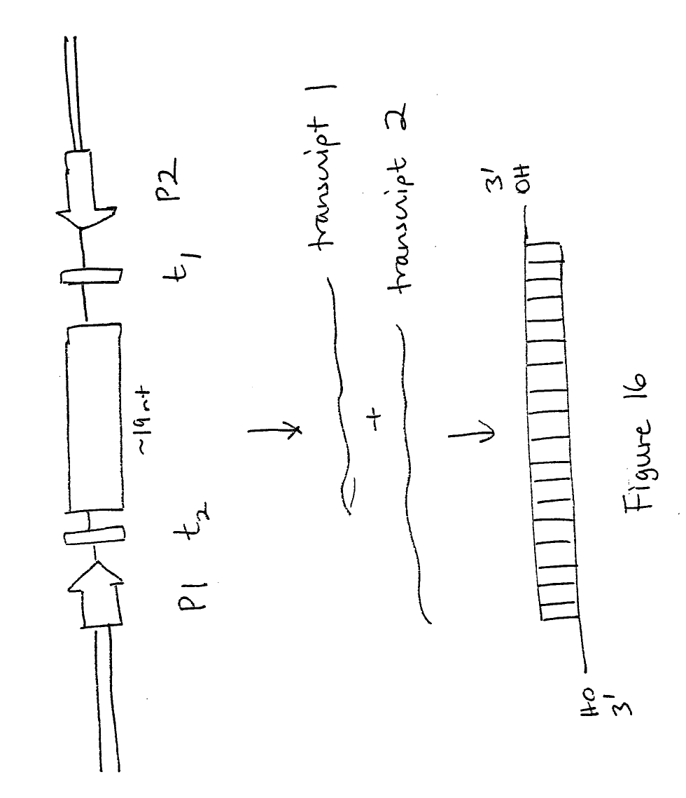

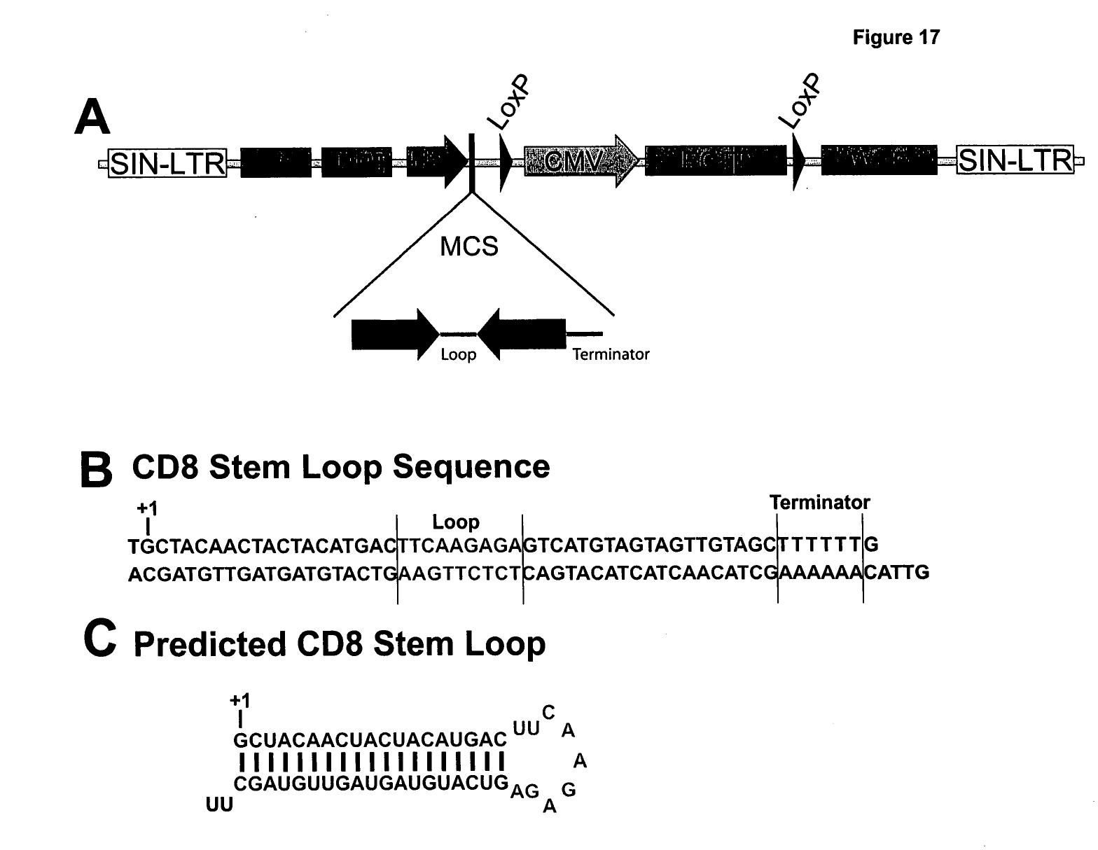

FIG. 17A schematically depicts the sequence of a nucleic acid comprising a segment which, when transcribed, produces an RNA that comprises two complementary elements that hybridize to one another to form a stem and a loop, inserted into the MCS of a lentiviral transfer plasmid of the invention. Complementary portions are indicated with arrows in opposite orientation to one another. FIG. 17B depicts a nucleic acid which, when transcribed, results in an RNA targeted to the CD8 molecule. FIG. 17C depicts the shRNA that results following hybridization of the complementary portions of an RNA transcribed from the nucleic acid in FIG. 17B. The RNA forms a stem-loop structure in which the stem is targeted to CD8. As described in more detail in Example 3, the inventors have shown that lentiviral transfer plasmids comprising a heterologous nucleic acid whose sequence includes the CD8 stem-loop sequence inhibit expression of CD8 when introduced into cells. In addition, as described in Examples 4 and 5, lentiviral particles comprising a heterologous nucleic acid whose sequence includes the CD8 stem-loop sequence inhibit expression of CD8 at both the mRNA and protein level when introduced into cells. Furthermore, the inhibition of expression persisted over the length of the experiment (1 month), demonstrating that RNAi mediated by the integrated lentivirus was stable. The inventors were unable to detect shRNA structures in the infected cells but were able to detect approximately 21 nucleotide-long RNAs comprising the CD8 stem loop sequence and having a typical siRNA structure. While not wishing to be bound by any theory, this results confirms the hypothesis that shRNAs are processed into siRNAs within the cell. The inventors also demonstrated that shRNA-mediated inhibition of CD8 was specific. In particular, shRNAs targeted to the mouse CD8 RNA did not inhibit expression of human CD8.

The invention therefore provides a lentiviral vector, e.g., a lentiviral transfer plasmid or lentiviral particle comprising a nucleic acid segment that provides a template for synthesis of one or more RNAs that self-hybridize or hybridize with each other to form an shRNA or siRNA, wherein the shRNA or siRNA is targeted to a target transcript and reduces expression of the transcript. For example, the invention provides a lentiviral transfer plasmid comprising the following elements: a nucleic acid including (i) a functional packaging signal; (ii) a multiple cloning site (MCS) into which a nucleic acid may be inserted; (iii) at least one additional element selected from the group consisting of: a second MCS, an HIV FLAP element, a heterologous promoter, a heterologous enhancer, an expression-enhancing posttranscriptional regulatory element, a target site for a site-specific recombinase, and a self-inactivating (SIN) LTR; and (iv) a nucleic acid segment that provides a template for synthesis of an shRNA or siRNA, which shRNA or siRNA is targeted to a target transcript and reduces expression of the transcript. In certain preferred embodiments of the invention the nucleic acid segment provides a template for synthesis of an RNA that self-hybridizes to form an shRNA.

Any of the various embodiments of the elements included in the lentiviral transfer plasmid or lentiviral particle may be selected as described above. In particular, the invention provides a lentiviral transfer plasmid comprising the following elements: a nucleic acid including (i) a functional packaging signal; (ii) a multiple cloning site (MCS) into which a nucleic acid may be inserted; (iii) a second MCS; (iv) an HIV FLAP element; (v) a WRE; (vi) two loxP sites; (vii) a self-inactivating (SIN) LTR; and (viii) a nucleic acid segment operably linked to a PolIII promoter, wherein the nucleic acid segment provides a template for synthesis of one or more RNAs that self-hybridize or hybridize with each other to form an shRNA or siRNA, which shRNA or siRNA is targeted to a target transcript and reduces expression of the transcript. In certain preferred embodiments of the invention the nucleic acid segment provides a template for synthesis of an RNA that hybridizes to form an shRNA.

Identification of sequences for design of the stem portion of an shRNA may be performed as described above for siRNAs. See also the Web sites having URLs www.mpibpc.gwdg.de/abteilungen/100/105/sirna.htrnl and katahdin.chsl.org:9331/RNAi (visited Oct. 23, 2002). The first step is to search for potential target sequences, e.g., by scanning the cDNA. According to certain embodiments of the invention a potential target sequence corresponds to any sequence of the form GN₁₈. According to other embodiments of the invention a potential target sequence corresponds to a sequence of the form AAGN₁₈. According to yet other embodiments of the invention a potential target sequence corresponds to a sequence of the form AAGN₁₈TT. Once a potential target is selected, the sequence GN₁₈ is used as the sequence for the stem (duplex) portion of the shRNA. Thus in certain embodiments of the invention the GN18 is preferably be surrounded by AA—TT in the context of the mRNA. Where the U6 promoter is used, a 5′ guanine is generally required due to the constraints of this promoter. It may be useful to test 4-5 targets for each transcript or gene of interest. It may be desirable to perform a database search (e.g., BLAST search) using the GN₁₈ sequences to verify that the sequence is unique in order to avoid silencing other genes in addition to the target gene. As lentivirus pseudotyped with VSV-G is capable of infecting human cells, if the lentivirus is not intended for use in humans it may be deisrable to determine if there are human genes that may be silenced. If so, it may be preferable to avoid sequences that would target such genes.

According to certain embodiments of the invention the sequence TTCAAGAGA (SEQ ID NO: 10) is selected for the loop. Thus to design the complete hairpin sequence according to certain embodiments of the invention, a 19 nt sequence suitable as the inhibitory portion of a typical siRNA is selected, optionally including an additional two nucleotides such as AA at the 5′ end in order to generate a 3′ UU overhang in the resulting shRNA. A loop sequence is added at the 3′ end of the 19 (or 21) nt sequence, followed by a sequence complementary to the 19 nt (or 21) sequence, resulting in a stem-loop after hybridization. See Example 3 for additional information. Any of a variety of other sequences may be selected for the loop including, but not limited to, loops used in the shRNAs described in Brummelkamp, et al., Paddison, et al., Sui, et al., Yui, et al., or Paul, et al.

The invention provides a method of reducing or inhibiting expression of a target transcript in a cell comprising: (i) delivering a lentiviral vector to the cell, wherein presence of the lentiviral vector within the cell results in transcription of one or more RNAs that self-hybridize or hybridize with each other to form an shRNA or siRNA that is targeted to the target transcript and reduces expression of the transcript. The lentiviral vector comprises a nucleic acid segment that provides a template for synthesis of one or more RNAs that self-hybridize or hybridize with each other to form an shRNA or siRNA that is targeted to the transcript and reduces expression of the transcript. In certain preferred embodiments of the invention the nucleic acid provides a template for synthesis of an RNA that hybridizes to form an shRNA.

According to certain embodiments of the invention the cell is a mammalian cell. Any of the lentiviral transfer plasmids or lentiviral particles described above may be used, wherein presence of the plasmid or particle in a cell provides a template for synthesis of one or more RNAs that self-hybridize or hybridize with each other to form a shRNA or siRNA targeted to the transcript of interest. According to certain embodiments of the invention the delivering step comprises delivering the lentiviral transfer plasmid or lentiviral particle to a mammalian subject, thereby delivering the lentiviral transfer plasmid or lentiviral particle to a cell that is present within the body of the subject. According to certain embodiments of the invention the cell is a primary cell. By “primary cell” is meant a cell that has been removed from the body of a subject and maintained in tissue culture for less than approximately 1, 2, 3, 4, or 5 doubling periods or a non-immortalized cell. According to certain embodiments of the invention the mammalian cell is a nondividing cell, e.g., a terminally differentiated T cell, neuron, hepatocyte, retinal cell, skeletal myocyte, cardiac myocyte, keratinocyte, macrophage, etc. The mammalian cell may be a human cell or a nonhuman (e.g., mouse or rat) cell. According to certain embodiments of the invention the mammalian cell is an embryonic cell or an embryonic stem cell.

The invention further provides a method for reversibly inhibiting or reducing expression of a target transcript in a cell comprising: delivering a lentiviral vector to the cell, wherein the lentiviral vector comprises a nucleic acid segment that provides a template for synthesis of one or more RNAs that self-hybridize or hybridize with each other to form an shRNA or siRNA, which shRNA or siRNA is targeted to the target transcript and reduces expression of the transcript, wherein the nucleic acid segment is located between sites for a site-specific recombinase; and (ii) inducing expression of the site-specific recombinase within the cell, thereby preventing synthesis of at least one of the RNAs. According to certain embodiments of the invention the cell is a mammalian cell. According to certain embodiments of the invention the recombinase is Cre. According to certain embodiments of the invention the step of inducing the site-specific recombinase comprises introducing a vector encoding the site-specific recombinase into the cell. According to other embodiments of the invention a nucleic acid encoding the site-specific recombinase is operably linked to an inducible promoter, and the inducing step comprises inducing the promoter within a cell containing the nucleic acid, as described above.

In addition, the invention provides a variety of methods for reversibly inhibiting or reducing expression of a target transcript in a conditional and/or tissue-specific manner. For example, the invention provides a method for reversibly inhibiting or reducing expression of a transcript in a mammal in a cell type specific or tissue-specific manner comprising: (i) delivering a lentiviral vector to cells of the mammal, wherein the lentiviral vector comprises a nucleic acid segment that provides a template for synthesis of one or more RNAs that self-hybridize or hybridize with each other to form an shRNA or siRNA, which shRNA or siRNA is targeted to the target transcript and reduces expression of the transcript, and wherein the nucleic acid segment is located between sites for a site-specific recombinase; and (ii) inducing expression of the site-specific recombinase in a subset of the cells of the mammal, thereby preventing synthesis of at least one of the RNAs within those cells. According to certain embodiments of the inventive methods the recombinase is Cre. According to certain embodiments of the invention the step of inducing the site-specific recombinase comprises introducing a vector encoding the site-specific recombinase into a subset of the cells of the subject, e.g., by utilizing a vector that requires a receptor present only on a subset of the cells. According to other embodiments of the invention a nucleic acid encoding the site-specific recombinase is operably linked to an inducible promoter, and the inducing step comprises inducing the promoter within cells containing the nucleic acid, as described above, whereby expression of the target transcript is restored only in cells or tissues in which the promoter is active.

In certain embodiments of the invention the nucleic acid encoding the site-specific recombinase is operably linked to a cell type or tissue-specific promoter, so that synthesis of the recombinase takes place only in cells or tissues in which that promoter is active, whereby expression of the target transcript is restored only in cells or tissues in which the promoter is active.

In certain preferred embodiments of the invention, the promoter utilized to direct expression of the one or more RNAs that self-hybridize or hybridize with each other to form an shRNA or siRNA is a promoter for RNA polymerase III (Pol III). Pol III directs synthesis of small transcripts that terminate within a stretch of 4-5 T residues. Certain Pol III promoters such as the U6 or H1 promoters do not require cis-acting regulatory elements (other than the first transcribed nucleotide) within the transcribed region and thus are preferred according to certain embodiments of the invention since they readily permit the selection of desired RNA sequences. In the case of naturally occurring U6 promoters the first transcribed nucleotide is guanosine, while in the case of naturally occurring H1 promoters the first transcribed nucleotide is adenine. (See, e.g., Medina MF and Joshi S., Curr Opin Mol Ther 1999 October;1(5):580-94; Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002)). Thus in certain embodiments of the invention, e.g., where transcription is driven by a U6 promoter, the 5′ nucleotide of preferred RNA sequences for formation of shRNAs or siRNAs is G. In certain other embodiments of the invention, e.g., where transcription is driven by an H1 promoter, the 5′ nucleotide may be A. The lentiviral transfer plasmid may be created by inserting a cassette comprising the RNA sequence into a transfer plasmid optimized for RNAi that already contains a suitable promoter, e.g., a plasmid such as pLL3.7. Alternately, a cassette comprising the RNA sequence operably linked to a suitable promoter may be inserted into a transfer plasmid that lacks such a promoter, e.g., a plasmid such as pLL3.0.

The invention thus encompasses administration of a lentiviral vector to a cell, e.g., a mammalian cell, to inhibit or reduce expression of any target transcript or gene, wherein the lentiviral vector comprises a nucleic acid segment that provides a template for synthesis of one or more RNAs that self-hybridize or hybridize to form an shRNA or siRNA that is targeted to the target transcript or gene. In general, the nucleic acid segment may provide a template for synthesis of any RNA structure capable of being processed in vivo to a shRNA or siRNA, so long as the RNA does not induce other negative events such as induction of the interferon response. In certain preferred embodiments of the invention the nucleic acid segment provides a template for synthesis of an RNA that self-hybridizes to form an shRNA targeted to the target transcript.

As discussed above, in addition to their use for synthesis of RNAs that self-hybridize to form shRNAs, the lentiviral vectors of the invention may be used for synthesis of various other RNAs that mediate RNAi. In particular, two separate approximately 21 nt RNA strands may be generated, each of which contains a 19 nt region complementary to the other, and the individual strands may hybridize together to generate an siRNA structure. Accordingly, the invention encompasses a lentiviral vector comprising two transcribable regions, each of which provides a template for synthesis of a transcript containing a region complementary with the other. Generally each transcript will be approximately 21 nt in length and the complementary regions will be approximately 19 nt in length, as described above.

In addition, the invention provides a lentiviral vector that contains oppositely directed promoters flanking a nucleic acid segment and positioned so that two different transcripts, approximately 21 nt in length and having complementary regions approximately 19 nt in length, are generated. It will be appreciated that appropriate terminators should be supplied in these cases. In cases in which the RNA structure undergoes one or more processing steps, those of ordinary skill in the art will appreciate that the nucleic acid segment will preferably be designed to include sequences that may be necessary for processing of the RNA. FIG. 16 presents a schematic diagram of such a plasmid.

A large number of variations on the above are possible. For example, the lentiviral vector may comprise multiple nucleic acid segments, each of which provides a template for synthesis of one or more RNAs that self-hybridize or hybridize with each other to form shRNAs or siRNAs, which shRNAs or siRNAs may target the same transcript or different transcripts. In addition, according to certain embodiments of the invention the nucleic acid segment provides a template for synthesis of a plurality of RNAs that self-hybridize or hybridize with each other to form a plurality of siRNAs or siRNA precursors. For example, a single promoter may direct synthesis of a single RNA transcript containing multiple self-complementary regions, each of which may hybridize to generate a plurality of stem-loop structures. These structures may be cleaved in vivo, e.g., by DICER, to generate multiple different siRNAs. It will be appreciated that such transcripts preferably contain a termination signal at the 3′ end of the transcript but not between the individual siRNA units.

The present invention encompasses any cell manipulated to contain an inventive lentiviral transfer plasmid, lentiviral particle, or lentiviral genome derived therefrom (e.g., a provirus), wherein the lentiviral transfer plasmid, lentiviral particle, or lentiviral genome provides a template for synthesis of one or more RNAs that self-hybridize or hybridize to form an shRNA or siRNA. Preferably, the cell is a mammalian cell. According to certain embodiments of the invention the cell is a human cell. Those of ordinary skill in the art will appreciate that intracellular expression of RNAs that self-hybridize or hybridize with each other to form shRNAs or siRNAs according to the present invention may allow the production of cells that produce the shRNA or siRNA over long periods of time (e.g., greater than a few days, preferably at least several months, more preferably at least a year or longer, possibly a lifetime).

In certain embodiments of the invention, the cells are non-human cells within an organism. For example, the present invention encompasses transgenic animals the cells of which contain an inventive lentiviral transfer plasmid, lentiviral particle, or lentiviral genome derived therefrom, wherein the lentiviral transfer plasmid, lentiviral particle, or lentiviral genome provides a template for synthesis of one or more RNAs that self-hybridize or hybridize to form an shRNA or siRNA in one or more cell types or tissues of the transgenic animal. The invention therefore provides a transgenic animal, one or more cells of which comprise a heterologous nucleic acid segment provided by a lentiviral vector, wherein the lentiviral vector comprises (i) a functional packaging signal; (ii) a multiple cloning site (MCS); and (iii) at least one additional element selected from the group consisting of: a second MCS, a second MCS into which a heterologous promoter or promoter-enhancer is inserted, an HIV FLAP element, an expression-enhancing posttranscriptional regulatory element, a target site for a site-specific recombinase, and a self-inactivating (SIN) LTR. According to certain embodiments of the invention the cells of the transgenic animal contain a heterologous nucleic acid segment that comprises sites for a site-specific recombinase.

As described in Example 7, the inventors have generated transgenic mice using a variety of lentiviral particles each comprising a first heterologous nucleic acid segment encoding the fluorescent protein GFP, and also a second heterologous nucleic acid segment that provides a template for synthesis of an RNA that self-hybridizes to form an shRNA targeted to a target transcript. The lentiviral particles were able to induce expression of GFP within embryonic stem cells (ES cells), and these ES cells gave rise to transgenic animals whose cells expressed GFP. Furthermore, expression of the particular target transcript corresponding to the second nucleic acid segment was reduced or inhibited in cells of the transgenic mice. These results demonstrate that the lentiviral transfer plasmids and lentiviral particles of the invention may be used to generate transgenic animals in which expression of a target transcript is reduced or inhibited. It is noted that the lentiviral vectors of the invention may thus generally be used a bifunctional vectors, leading both to expression of a heterologous nucleic acid and silencing of an endogenous gene.

Kits

The invention provides a variety of kits comprising one or more of the lentiviral transfer plasmids of the invention. For example, the invention provides a kit comprising (a) a lentiviral transfer plasmid comprising a nucleic acid sequence including (i) a functional packaging signal; (ii) a multiple cloning site (MCS) into which a heterologous gene may be inserted; and (iii) at least one additional element selected from the group consisting of: a second MCS, an HIV FLAP element, a heterologous promoter, a heterologous enhancer, an expression-enhancing posttranscriptional regulatory element, a target site for a site-specific recombinase, and a self-inactivating (SIN) LTR; and one or more of the following items: (i) a packaging mix comprising one or more plasmids that collectively provide nucleic acid sequences coding for retroviral or lentiviral Gag and Pol proteins and an envelope protein. The packaging mix may contain two or more plasmids. According to certain embodiments of the invention the packaging mix includes two plasmids, one of which provides nucleic acid sequences coding for Gag and Pol and the other of which provides nucleic acid segments coding for an envelope protein; (ii) cells (e.g., a cell line) that are permissive for production of lentiviral particles such as 293T cells; (iii) packaging cells, e.g., a cell line that is permissive for production of lentiviral particles and provides the proteins Gag, Pol, Env, and, optionally, Rev; (iv) cells suitable for use in titering lentiviral particles; a transfection-enhancing agent such as Lipofectamine; (v) a selection agent such as an antibiotic, preferably corresponding to an antibiotic resistance gene in the lentiviral transfer plasmid; (vi) instructions for use; (vii) a lentiviral transfer plasmid comprising a heterologous nucleic acid segment such as a reporter gene that may serve as a positive control (referred to as a “positive control plasmid”).

According to certain embodiments of the invention the kit contains a set of lentiviral transfer plasmids comprising a variety of different heterologous promoters and/or reporter genes. For example, the kit may contain a set of two or more vectors selected from the group consisting of the plasmids of: SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

Therapeutic Applications and Pharmaceutical Formulations

The lentiviral vectors of the invention are useful for a wide variety of therapeutic applications. In particular, they are useful in any context for which gene therapy is contemplated. For example, lentiviral vectors comprising a heterologous nucleic acid segment operably linked to a promoter are useful for any disease or clinical condition associated with reduction or absence of the protein encoded by the heterologous nucleic acid segment, or any disease or clinical condition that can be effectively treated by augmenting the expression of the encoded protein within the subject. For example, lentiviral vectors comprising a nucleic acid segment encoding the cystic fibrosis transmembrane conductance regulator (CFTR) or encoding a α1-antitrypsin may be used for the treatment of cystic fibrosis and α1-antitrypsin deficiency, respectively. Lentiviral vectors comprising a nucleic acid segment encoding Factor VIII or Factor IX may be used for treatment of hemophilia A or B, respectively. See the Web site having URL www.wiley.co.uk/genetherapy/clinical/(visited Oct. 19, 2002) for a representative list of current gene therapy clinical trials involving expression of a therapeutic protein in a subject in need of treatment.

Inventive lentiviral vectors capable of causing intracellular synthesis of inhibitory RNAs (siRNAs or shRNAs) are useful in treating any disease or clinical condition associated with overexpression of a transcript or its encoded protein in a subject, or any disease or clinical condition that may be treated by causing reduction of a transcript or its encoded protein in a subject. For example, many cancers are associated with overexpression of oncogene products. Delivering a lentiviral vector that provides a template for synthesis of one or more RNAs that self-hybridize or hybridize with each other to form an shRNA or siRNA targeted to the transcript encoding the oncogene product may be used to treat such cancers. The high degree of specificity achieved by RNA interference suggests that it is possible to selectively target transcripts containing single base pair mutations while not interfering with expression of the normal cellular allele. Lenviral vectors that provide a template for synthesis of one or more RNAs that self-hybridize or hybridize with each other to form an shRNA or siRNA targeted to a transcript encoding a cytokine may be used to regulate immune system responses (e.g., responses responsible for organ transplant rejection, allergy, autoimmune diseases, inflammation, etc.). Lentiviral vectors that provide a template for synthesis of one or more RNAs that self-hybridize or hybridize with each other to form an shRNA or siRNA targeted to a transcript of an infectious agent or targeted to a cellular transcript whose encoded product is necessary for or contributes to any aspect of the infectious process may be used in the treatment of infectious diseases.

Gene therapy protocols may involve administering an effective amount of a lentiviral vector whose presence within a cell results in production of a therapeutic siRNA or shRNA to a subject either before, substantially contemporaneously, with, or after the onset of a condition to be treated. Another approach that may be used alternatively or in combination with the foregoing is to isolate a population of cells, e.g., stem cells or immune system cells from a subject, optionally expand the cells in tissue culture, and administer a lentiviral vector whose presence within a cell results in production of a therapeutic siRNA or shRNA to the cells in vitro. The cells may then be returned to the subject, where, for example, they may provide a population of cells that produce a therapeutic shRNA or siRNA, or that are resistant to infection by an infectious organism, etc. Optionally, cells expressing the therapeutic shRNA or siRNA can be selected in vitro prior to introducing them into the subject. In some embodiments of the invention a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Compositions comprising lentiviral vectors of the invention may provide a template for a single siRNA or shRNA species, targeted to a single site in a single target transcript, or alternatively may provide templates for a plurality of different siRNA or shRNA species, targeted to one or more sites in one or more target transcripts. In some embodiments of the invention, it will be desirable to utilize compositions comprising one or more lentiviral vectors, wherein presence of the lentiviral vector(s) within a cell or within different cells in the body, results in production of a plurality of different siRNA or shRNA species targeted to different genes, which may be cellular genes or, where an infection is being treated, genes of an infectious organism. Also, some embodiments will provide templates for more than one siRNA or shRNA species targeted to a single transcript. To give but one example, it may be desirable to provide templates for synthesis of one or more RNAs that self-hybridize or hybridize with each other to form at least one siRNA or shRNA targeted to coding regions of a target transcript and at least one siRNA or shRNA targeted to the 3× UTR. This strategy may provide extra assurance that products encoded by the relevant transcript will not be generated because at least one siRNA or shRNA will target the transcript for degradation while at least one other inhibits the translation of any transcripts that avoid degradation. The invention encompasses “therapeutic cocktails”, including approaches in which a single lentiviral particle provides templates for synthesis of one or more RNAs that self-hybridize or hybridize to form shRNAs or siRNAs that inhibit multiple target transcripts.

It may be desirable to combine the administration of inventive lentiviral vectors with one or more additional therapeutic agents. The invention therefore encompasses compositions comprising a lentiviral vector of the invention, preferably a lentiviral particle, and a second therapeutic agent, e.g., a composition approved by the U.S. Food and Drug Administration.

Inventive compositions may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include a lentiviral vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive lentiviral vectors are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment, the active agents, i.e., a lentiviral vector of the invention and/or other agents to be administered together with a lentiviral vector of the invention, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions such as HIV it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a lentiviral vector as described herein, can include a single treatment or, in many cases, can include a series of treatments.

Exemplary doses for administration of gene therapy vectors are known in the art. It is furthermore understood that appropriate doses of a lentiviral vector that provides a template for synthesis of one or more RNAs that self-hybridize or hybridize with each other to form an shRNA or siRNA may, in general, depend upon the potency of the siRNA or shRNA and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

Lentiviral gene therapy vectors can be delivered to a subject by, for example, intravenous injection, local administration, or by stereotactic injection (see e.g., Chen et al. (1994) Proc. Natl. Acad. Sci. USA 91:3054-3057). In certain embodiments of the invention the vectors may be delivered orally or inhalationally and may be encapsulated or otherwise manipulated to protect them from degradation, enhance uptake into tissues or cells, etc. The pharmaceutical preparation can include the lentiviral vector in an acceptable diluent, or can comprise a slow release matrix in which the lentiviral vector is imbedded. Alternatively, where the vector can be produced intact from recombinant cells, as is the case for retroviral or lentiviral vectors as described herein, the pharmaceutical preparation can include one or more cells which produce the vectors.

Inventive pharmaceutical compositions can be included in a container, pack, or dispenser together with instructions for administration.

### Exemplification

### EXAMPLE 1

### Generation of pLentiLox Vectors

This example describes generation of the pLentiLox family of vectors. Unless otherwise indicated, standard molecular biology techniques were generally performed in accordance with guidance found in Current Protocols in Molecular Biology, edition as of 2001; or in Sambrook, Russell, and Sambrook, Molecular Cloning: A Laboratory Manual, 3^{rd} ed., Cold Spring Harbor Laboratory Press, Cold Spring Harbor, 2001, or according to instructions provided by the manufacturer of the relevant reagents or kits. It is noted that a variety of different approaches to generating the constructs described below as well as alternative sources for the elements incorporated into the constructs may be employed. In particular, the sequence information provided herein enables one of ordinary skill in the art to chemically synthesize part or all of the constructs, thus offering considerable flexibility.

Characterization of pBFGW.

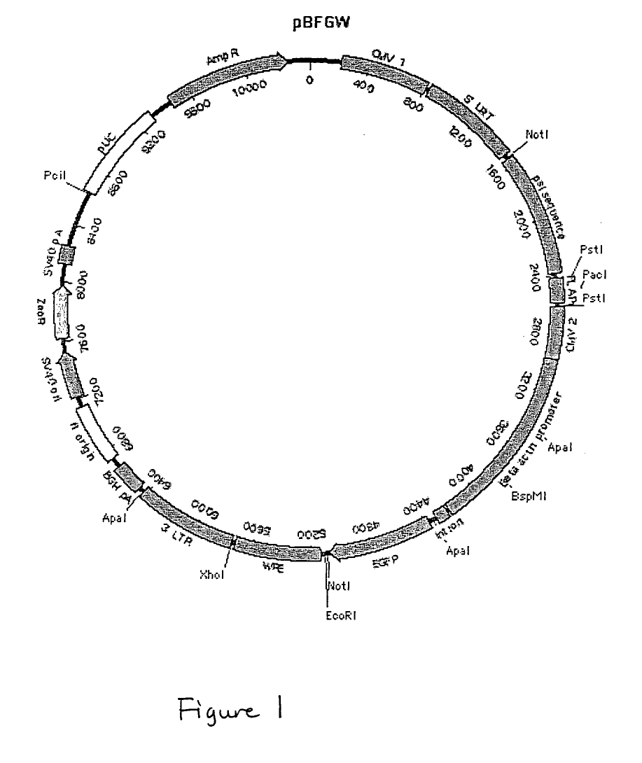

Generation of the pLentiLox vector family involved extensive modification of the plasmid pBFGW. Accordingly, the first step was a thorough characterization of this plasmid. pBFGW is a third generation lentiviral plasmid based upon the pCDNA 3.1/Zeo plasmid (Invitrogen) that was incompletely characterized and lacked sequence information. pBFGW is a member of the same vector family as pFUGW²⁴ but contains a Beta-actin/CMV hybrid promoter rather than a ubiquitin promoter. Lentiviral elements in pBFGW are derived from HIV-1. We sequenced this plasmid in its entirety. The sequence is presented as SEQ ID NO: 1. A restriction map of pBFGW was generated based upon the sequencing information and verified (for several enzymes) by digestion and agarose gel electrophoresis and is shown in FIG. 1.

pBFGW includes a cassette for the generation of lentivirus inserted downstream of the CMV promoter of pCDNA3.1/Zeo. The cassette consists of a 5′ self-inactivating (SIN) LTR, the required packaging sequence (Psi) , the HIV FLAP element (FLAP), a hybrid promoter consisting of beta-actin and CMV promoter sequences, the open reading frame for enhanced Green Fluorescent Protein (EGFP), the Woodchuck Hepatitis Regulatory Element (WRE), and the 3′ SIN LTR. There existed only three unique restriction sites for the introduction of transgenes and/or promoters. In addition pBFGW was 10,441 base pairs in length without the introduction of a transgene. Plasmids of this size are more difficult to manipulate then smaller plasmids. There was also no mechanism to eliminate transgene expression after infection.

Elimination of Elements Between FLAP and WRE.

pBFGW was sequentially digested with PacI and EcoRI. The 7,930 bp fragment representing the backbone was purified by gel purification. The overhanging ends were filled-in by reaction with Pfu polymerase to generate blunt ends.

Introduction of A MCS-LoxP-MCS-LoxP Cassette.

The plasmid pBluescript Lox (pBS-Lox) was created by amplifying two LoxP sites from an unrelated vector (pML2MIG) by PCR. The two LoxP PCR products contained a 23 bp overlap region (containing the restriction sites EcoRI, NotI and HindIII) at their 5′ and 3′ ends respectively. These two products were combined in a splicing overlap extension (SOE) PCR reaction (Horton, R M, et al., Biotechniques, 8(5):528-35, 1990) using standard PCR conditions to create the LoxP-MCS-LoxP cassette, that was cloned into the filled-in HindIII and NotI sites of pBlueScript (KSII+) as a blunt ended fragment. The following primers were used in the PCR reactions:

PCR product #1 was amplified with the following primers:

L1/5′: 5′ (tggtgggtacctagtggaacc)3′ (SEQ ID NO: 32)

L1/3′: 5′ (aagcttaagcggccgcagaattcgtcgagggacctaataacgtatag)3′ (SEQ ID NO: 33)

PCR product #2 was amplified with the following primers:

L2/5′: 5′ (gaattctgcggccgcttaagcttggaacccttaatataacttcg)3′ (SEQ ID NO: 34)

L2/3′: 5′ (cgcttcacgagattccagcag)3′ (SEQ ID NO: 35)

The LoxP-MCS-LoxP cassette was used in the LentiLox cloning described below. The second LoxP site of this cassette contained a three base pair deletion. This deletion was not identified until later in the construction of the LentiLox vectors (see below).

pBS-Lox was digested with Asp718. A 209 bp fragment was isolated by gel purification. This fragment contained a 5′ multiple cloning site (5′ MCS), a LoxP site, a 3′ multiple cloning site (3′ MCS), and a second LoxP site. The overhanging ends of the 209 bp Asp718 fragment were filled-in with cloned Pfu polymerase, and ligated into the 7,930 bp fragment of pBFGW. The orientation of the insertion was determined both by restriction fragment length polymorphism and by sequencing. A plasmid containing the MCS-LoxP-MCS-LoxP cassette with the expected sequence in the correct orientation was named pLentiLox 1.0.

Elimination of Plasmid Backbone Restriction Sites.

Several of the sites found within the two MCS's were present elsewhere in the pLentiLox 1.0 vector. Specifically, NotI, Apal, and XhoI cut both within the MCS cassette as well as once elsewhere in the vector. In order to gain the use of these sites we intended to destroy each site within the plasmid backbone.

pLL1.0 was partially digested with NotI under conditions of limiting enzyme activity (0.0625 Units of enzyme per microgram of pLL1.0 incubated at 37 degrees Celsius for twenty minutes). The 8,142 bp band that represented linearized pLL1.0 was isolated via agarose gel electrophoresis followed by gel extraction. This linearized fragment was phosphorylated on its 5′ ends with T4 Polynucleotide Kinase (PNK). The overhanging ends of this molecule were then filled-in with cloned Pfu to destroy the NotI site. The ends were ligated together to circularize the molecule. To determine whether a NotI site had been destroyed and to determine which NotI site had been destroyed the plasmid was digested with NotI and with PstI. Destruction of the NotI site in the plasmid backbone yielded fragments of 7830 bp and 312 bp whereas destruction of the MCS NotI site yielded fragments of 7253 and 889 bp respectively. We accidentally chose a plasmid in which the MCS NotI site was destroyed and named it pLentiLox 1.1 (pLL1.1). This was later remedied (see below). It should be noted that the creation of pLL1.1 was problematic due to recombination within the vector resulting in large deletions of required sequences. To verify the presence of an intact backbone it was necessary to perform an additional restriction digest. An enzyme that cut in three places was used, and the digestion pattern of pLL1.1 was compared with that of pLL1.0 to make sure that the two bands representing the backbone were the same size.

We designed a strategy to destroy the Apal site that would also eliminate a 2197 bp fragment between the 3′SIN LTR and the pUC ori that we deemed non-essential for lentiviral production. This ApaI-PciI fragment contained a BGH polyadenylation site, an SV40 promoter/ori, the Zeomycin resistance gene, and an SV40 polyadenylation site. We digested pLL1.1 with PciI to linearize the plasmid. The linearized plasmid was then digested with a limiting amount of ApaI (between 0.25 Units and 2 Units per microgram of linearized pLL1.1 for twenty minutes at room temperature). A 5,945 bp fragment representing a single cut with ApaI adjacent to the 3′ LTR was isolated by agarose gel electorphoresis followed by gel purification. The gel purified fragment was phosphorylated with PNK, filled in with cloned Pfu, and circularized by ligation. The ligated DNA was digested with StuI prior to transformation into bacteria. Digestion with Stul was expected to specifically cut plasmid that contains the 2197 bp fragment that had been eliminated, and thus was used to select against contamination with uncut pLL1.1 vector. The elimination of the 2197 bp and the destruction of PciI and ApaI were verified by restriction digest and a correct plasmid was identified and named pLentiLox 1.2 (pLL1.2).

To destroy the XhoI site, pLL1.2 was cut with limiting amounts of XhoI (0.0625 Units per microgram of plasmid for twenty minutes at 37 degrees Celsius). A 5,947 bp fragment representing single-cut linearized pLL1.2 was isolated via agarose gel electrophoresis and gel purification. The fragment was 5′ phosphorylated with PNK, filled in with cloned Pfu, recircularized with ligase, and transformed into bacteria. Destruction of the correct XhoI site was verified by restriction digest. A correct plasmid was identified and named pLentiLox1.3 (pLL1.3).

Expansion of the 5′ MCS.

The 5′ MCS was intended for the insertion of promoter sequences, among other purposes. After destruction of the sites mentioned above two unique cloning sites (ApaI and XhoI) remained in this MCS. We derived a list of restriction enzymes that failed to cut pLL1.3 to generate a list of candidate sites to engineer into an expanded 5′MCS. We then designed complementary oligonucleotides to allow us to introduce XbaI, HpaI, NheI, and PacI sites between the ApaI and XhoI sites. The oligonucleotides were designed to include two nucleotides between adjacent restriction sites. The sequence of the sense oligonucleotides was 5′ cgctctagacggttaacgcgctagccgttaattaagcc 3′ (SEQ ID NO: 11). The antisense oligonucleotide was complementary to this sequence but contained an additional four nucleotides at the 5′ end to produce an XhoI overhang and four nucleotides at the 3′ end to produce an ApaI overhang. The antisense oligonucleotide sequence was 5′-tcgaggcttaattaacggctagcgcgttaaccgtctagagcgggcc-3′ (SEQ ID NO: 12). We chose restriction sites to include based upon the following criteria: (1) Inclusion of a site for a restriction enzyme that leaves a blunt end after cutting; (2) Inclusion of a restriction site that has an 8 bp recognition sequence. (3) Inclusion of sites for which enzymes are widely available. (4) Inclusion of sites for enzymes that are known to be reliable cutters.

pLL1.3 was digested sequentially with ApaI and XhoI. The digest was then purified by Qiaquick PCR purification kit (Qiagen) to eliminate the small DNA fragment between Apal and XhoI. The fragment was then treated with Shrimp Alkaline Phosphatase (SAP) to eliminate 5′-phosphate groups. The oligonucleotides described above were synthesized, 5′ phosphorylated, and PAGE-purified by IDT Corp. (See Web site having URL www.idtna.com. 60 picomols of each oligo were annealed in annealing buffer (100 mM Potassium Acetate, 30 mM HEPES-KOH pH 7.4, 2 mM Magnesium acetate) by incubation at 95 degrees for 4 minutes, followed by 70 degrees for 10 minutes, then slowly cooled (0.1 degrees/second) to 4 degrees, then maintained at 4 degrees for 10 minutes. The annealed oligos were diluted and ligated at an equimolar concentration with the linearized pLL1.3 vector. A plasmid containing the engineered MCS was identified by restriction digest and named pLL1.4.

It was at this time that we first realized that we had destroyed the NotI site in the MCS rather than in the plasmid backbone (see above). The second NotI site (adjacent to the LTR) was then destroyed in pLL1.4 as follows. pLL1.4 was digested with NotI to linearize the plasmid. The ends were 5′ phosphorylated with PNK and were filled-in with cloned Pfu to blunt and destroy the NotI site. The plasmid was recircularized by ligation and transformed into bacteria. We checked for destruction of the NotI site by restriction digest and named the resulting plasmid pLentiLox 1.5 (pLL1.5).

We next sought to expand the 3′ MCS. We designed primers to introduce NsiI, SphI, SmaI/Xmal, AscI, and BamHI sites between the NotI and EcoRI sites. We followed the same design criterion as described above. An additional criterion was that no three consecutive nucleotides would generate a nonsense codon. This would allow us to produce fusion proteins in which MCS sequence can remain between the fused proteins while minimizing the likelihood of premature termination of translation. In addition we wanted a minimum of sites in the MCS to be present in the sequences encoding EGFP and dsRed2, which we intended to include in many derivatives of our vectors (see below). We purposefully intended to make this MCS more versatile then the 5′ MCS since we anticipated that most applications of our vector would require cloning into this MCS. The inclusion of an SphI site was fortuitous. The two nucleotide spacer between the NsiI and SmaI/XmaI led to the creation of an SphI site that overlaps these other two sites. More fortuitously, SphI is a unique site in pLL1.5. The oligonucleotide primers incorporating the desired restrictions sites are as follows:

3′ MCS Sense: 5′ ggccgccgatgcatgccccgggatggcgcgccatggatccgcg 3′ (SEQ ID NO: 13)

3′ MCS Antisense: 5′ aattcgcggatccatggcgcgccatcccggggcatgcatcggc 3′ (SEQ ID NO: 14)

Because we had destroyed the NotI site that should have been present in the pLL1.5 3′ MCS we had to use a different strategy to insert the oligonucleotides than was used for the 5′ MCS. The pBS-Lox (described above) plasmid was digested with Noti and EcoRI enzymes. The small DNA fragment that was liberated was eliminated by purifying the linearized pBS-Lox backbone in a Qiaquick PCR purification kit. The DNA was SAP treated. The 3′ MCS oligonucleotides were annealed (see above). 150 finols of annealed oligonucleotides and cut pLL1.5 were ligated together and transformed into bacteria. A plasmid containing the expanded 3′ MCS was identified by restriction digest and named pBS-Lox-MCS.

To insert the expanded MCS from pBS-Lox-MCS into pLL1.5, we replaced the EcoRI-XhoI fragment from pLL1.5 (containing the improperly destroyed NotI site) with the EcoRI-XhoI fragment from pBS-Lox-MCS (containing an intact NotI site and the expanded 3′ MCS). A plasmid containing the expanded MCS and intact NotI in the pLL backbone was identified by restriction digest and was named pLentiLox2.0 (pLL2.0).

Production of Useful pLL2. 0 Series Vectors

We next sought to produce constructs that would be useful starting points for many potential uses of our lentiviral vectors. One application in which there is enormous interest is the generation of fusion proteins in which a protein of interest (or a portion thereof) is fused with a fluorescent protein. In particular, EGFP and dsRed2 are fluorescent proteins that are well characterized and for which sequences are widely available.

pLL2.1 was engineered to include the EGFP open reading frame. The EGFP open reading frame was amplified from pEGFP-N1 (Clontech) to include a 5′NotI site and a 3′NsiI site. The oligonucleotides used were:

EGFP/5′NotI: 5′-cggcggccgcgccaccatggtgagcaagggc-3′ (SEQ ID NO: 15)

EGFP/3′NsiI: 5′-cgatgcatcttgtacagctcgtccatgccg-3′ (SEQ ID NO: 16)

The PCR product was isolated by agarose gel electrophoresis, gel purified, and cloned into the NotI and NsiI sites of pLL2.0 to create pLL2.1.

pLL2.2 was engineered to include the dsRed2 open reading frame. The dsRed2 open reading was amplified from pdsRed2-N1 (Clontech) to include a 5′NotI site and 3′NsiI site. The oligonucleotides used were:

dsRed2/5′NotI: 5′-cggcggccgcgccaccatggcctcctccgag-3′ (SEQ ID NO: 17)

dsRed2/3′NsiI: 5′-cgatgcatcaggaacaggtggtggcggccc-3′ (SEQ ID NO: 18)

The PCR product was isolated by agarose gel electorpheresis, gel purified, and cloned into the NotI and NsiI sites of pLL2.0 to create pLL2.2.

Because the pLentiLox series has a self-inactivating 5′ LTR, the provirus has no endogenous 5′ promoter activity. Therefore, it is necessary to include an internal promoter to drive transgene expression. This makes the system compatible with tissue-specific promoters. We chose to clone a ubiquitous and constitutive promoter into our vector to create a transgenic system that should be active in most eukaryotic cell types as well as in all the tissues of a mouse. The promoter we chose was the Ubiquitin C promoter (UbC). We first attempted to clone UbC by PCR amplification of the promoter from the pUB6/V5/His vector. However, the UbC sequence was not robustly amplified via PCR (in several attempts). As a second strategy we digested the pUB6/V5/His vector with BglII and HindIII which generates a fragment containing the UbC promoter. This fragment was isolated by agarose gel electrophoresis and gel extraction. The fragment was filled-in with cloned Pfu polymerase and 5′ phosphorylated with PNK. This fragment was ligated into HpaI digested pLL2.0, pLL2.1, and pLL2.2 to generate pLL2.3, pLL2.4, and pLL2.5 respectively. These plasmids were verified by restriction digest to contain the proper insert. pLL2.4 and pLL2.5 were transfected into 293.T cells and production of the correct fluorescent protein was verified by visualizing the transfected cells under an epifluorescent microscope 24 hours after transfection.

As described in the following example, we later discovered that one of the loxP sites in the pLL2.0 series of vectors contained a deletion that rendered it unusable. In order to generate the pLL3.0 series (which contain two wild type loxP sites) from the pLL2.1-pLL2.7 series we cloned ApaI-EcoRI inserts of various sizes (2.1-917 bp, 2.2-875bp, 2.3-1417 bp, 2.4-2,138 bp, 2.5-2096 bp, 2.6-1519 bp, and 2.7-1819 bp) from the pLL2.1-pLL2.7 vectors into the 5,831 bp ApaI-EcoRI backbone from pLL3.0 to create pLL3.1-pLL3.7.

### EXAMPLE 2

### Generation of Lentiviral Vectors for RNAi

Modification of pLL2.0 for Use in RNAi

In order to drive expression of an RNAi-inducing stem-loop, we decided to incorporate a polIII promoter into a pLentilox series vector. In addition, we decided to incorporate a polII promoter to drive expressin of EGFP as a reporter. Because we were concerned that the placement of a strong polII promoter near a polIII promoter might interfere with poli function we chose to place the polII-EGFP cassette between LoxP sites. This would allow us to eliminate the polII promoter if we were failing to accumulate the stem-loop RNA.

We first inserted a cassette to drive expression of EGFP. A DNA fragment containing the CMV promoter upstream of the EGFP open reading frame was amplified from pEGFP-C1. The oligonucleotides were selected to engineer a 5′ NotI site and 3′ EcoRI site. The oligonucleotides used were:

5′CMV/NotI: 5′-cggcggccgcgtggataaccgtattaccgccatg-3′ (SEQ ID NO: 19)

3′EGFP/stop/EcoRI: 5′ cggaattcctacttgtacagctcgtccatgccgag-3′ (SEQ ID NO: 20) The PCR product was isolated by agarose gel electrophoresis and purified by gel extraction. The fragment was cloned into the NotI and EcoRI sites of pLL2.0 to create pLL2.6. This plasmid was tested by restriction digest and production of EGFP was verified by transfection of pLL2.6 into 293.T cells.

The insertion of the U6 promoter presented an additional challenge. We needed to introduce a cloning site for the introduction of RNAi sequences. The U6 promoter includes sequences required for activity up until the +1 transcriptional start site. Therefore, one cannot modify the sequences prior to −1 without incapacitating U6. We did not want to introduce a site after +1 as that would dictate that the first several nucleotides of the stem-loop would be derived from the restriction site. We therefore engineered the U6 promoter to introduce an HpaI site that cuts at the −1 position of U6. The first three nucleotides of the HpaI site are present in the wildtype U6. We had to alter the nucleotides at −1 to +2 in order to engineer an HpaI site. As a result the U6 promoter is not functional when containing the HpaI site. However after digestion with HpaI and introduction of oligonucleotides to code for a stem-loop, those oligonucleotides can re-generate the wild-type 3′end of the U6 promoter thereby restoring transcriptional activity. We engineered oligonucleotides to add a XbaI site to the 5′ end of the U6 promoter and HpaI, BstEII, and XhoI sites to the 3′ end. We cloned the amplified PCR product from the pmU6 plasmid and introduced the product into the XbaI and XhoI sites of pLL2.6. The oligonucleotides used were:

5′ XbaI/U6: 5′-gctctagagatccgacgccgccatctctag-3′ (SEQ ID NO: 21)

3′ XhoI/BstEII/HpaI/U6: 5′-gcctcgagggtcaccgcgcgttaacaaggcttttctccaaggg-3′ (SEQ ID NO: 22)

The resulting plasmid was verified by both restriction digest and by sequencing and was named pLL2.7.

Repair of LoxP and Engineering of New Restriction Site

It was at this point that we recognized that the 3′ LoxP site in the original pBS-Lox contained a three nucleotide deletion that rendered it unusable. We decided to fix the 3′ LoxP site in the pLL2.0 plasmid and then use this plasmid backbone to clone in sequences from pLL2.1-pLL2.7. The repair of the LoxP site gave us an opportunity to engineer a new restriction site outside of the LoxP site (between the 3′ LoxP and the WRE). This site would give our plasmid series even greater flexibility for engineering other additions such as IRES-GFP, or inducible expression systems.

Oligonucleotides were designed to amplify a fragment from pLL2.0 from the EcoRI site to a PflMI site located within WRE. The 5′ oligonucleotide extended from the EcoRI site in the 3′MCS through the mutant LoxP site and into the region between the LoxP and the WRE. This oligonucleotide was designed to add the deleted nucleotides to the LoxP site and to create a PciI site immediately following the LoxP site. The amplified DNA was inserted into the pLL2.0 backbone digested with EcoRI and PflMI. The oligonucleotides used were:

5′EcoRI/LoxFix/PciI: 5′-gcgaattcgtcgagggacctaataacttcgtatagcatacattatacgaagttatacatgtttaagggttccgg-3′ (SEQ ID NO: 23)

| | |
|---|---|
| 3′ Pf1M1/Rev: | |
| 5′-aaggagctgacaggtggtggcaatg-3′ | (SEQ ID NO:24) |

A plasmid was checked by sequencing for the addition of a correct LoxP and PciI sites and named pLL3.0.

In order to generate the pLL3.1-pLL3.7 series from the pLL2. 1-pLL2.7 series we cloned ApaI-EcoRI inserts of various sizes (2.1-917 bp, 2.2-875 bp, 2.3-1417 bp, 2.4-2,138 bp, 2.5-2096 bp, 2.6-1519 bp, and 2.7-1819 bp) from the pLL2.1-pLL2.7 vectors into the 5,831 bp ApaI-EcoRI backbone from pLL3.0 to create pLL3.1-pLL3.7. All plasmids were verified by restriction digest.

### EXAMPLE 3

### Specific Silencing of Genes in T Cells using a Lentiviral Vector

Materials and Methods

Cell Culture:

E10 and primary mouse splenocyte cultures were performed as previously described (11). 293T cells (human fibroblasts) were cultured as described (21). In vitro T-cell proliferation was performed on 200,000 activated T-cells cultured in the presence/absense of increasing doses of IL2 (0 to 100 ng/ml) and pulsed for 6 h with [³H]TdR to assay proliferation.

Oligonucleotide Design.

The following approach was used to design oligonucleotides suitable for cloning into pLL3.7 vectors to generate vectors capable of directing synthesis of shRNAs for gene silencing in this and the following examples. As described above, we have engineered a multiple cloning site immediately following the U6 promoter. An HpaI site leaves a blunt end prior to the −1 position in the promoter. The oligonucleotide design must incorporate a 5′ T in order to reconstitute the −1 nucleotide of U6. An XhoI site cuts downstream of the U6 start site. The following oligonucleotide format was used:

Sense oligonucleotide:
5′T-(GN18)-(TTCAAGAGA)-(81NC)-TTTTT

Antisense oligonucleotide: Complement of sense but with additional nucleotides at 5′ end to generate XhoI overhang.
The loop sequence (TTCAAGAGA) (SEQ ID NO: 10) is based upon Brummelkamp et al. (Science 2002).
Oligonuclotides with 5′ phosphates and PAGE purified were ordered from Integrated DNA Technologies (IDT), Coralville, IA.

Generation of lentiviral transfer plasmids containing shRNAs targeted to CD8.

Oligonucleotides having the following sequences were inserted into pLL3.7 to produce lentiviral transfer plasmids capable of directing expression of an shRNA targeted to CD8.

| | |
|---|---|
| CD8 sense: | |
| 5′-tgctacaactactacatgacttcaagagagtca | (SEQ ID NO:36) |
| tgtagtagttgtagcttttttg-3′ | |
| CD8 antisense: | |
| 5′-gttacaaaaaagctacaactactacatgactct | (SEQ ID NO:37) |
| cttgaagtcatgtagtagttgtagca-3′ | |

The following protocol was used to clone oligonucleotides into pLL3.7 in this and the following examples:

Oligos are resuspended in water at 60 pmol/λ and annealed as follows.

Annealing oligos:

1λ Sense oligo
1λ Antisense oligo
48λ Annealing Buffer
   Annealing Buffer Recipe:
100 mM K-acetate
30 mM HEPES-KOH pH 7.4
2 mM Mg-acetate
Incubate at 95° 4 min
   70° 10 min
Decrease temperature to 4° slowly (0.1° C./min)
Incubate at 4° 10 min
   pLentiLox 3.7 is digested as follows:
Digest 1-2 μg with XhoI and HpaI
Treat with SAP or with CIP
Purify linearized fragment
Estimate concentration
   Ligation is performed as follows:
Ligate linearized product and annealed oligos at equimolar concentration. I typically use 60 fmol of each component in a final concentration of 10 μL.
Transformation is performed according to standard techniques. The use of an end A⁻ strain of E. coli, e.g., STBL-2 cells is strongly recommended.
   Clones are tested for the presence of inserts as follows:
We have had success testing for insertion of the stem-loop sequence with both colony pcr or by restriction digest. Insertion of insert causes a band shift of ˜60 bp in an XbaI/NotI fragment when compared to parental vector. This can be seen by 2% agarose gel electrophoresis. RPMI. Cell viability immediately after electroporation was typically around 60%.

Electroporation:

For electroporations, 10 μg of LentiLox plasmid were added to prechilled 0.4-cm electrode gap cuvettes (Bio-Rad, Hercules, Calif.). E10 cells (1.5×10⁷) were resuspended to 3×10⁷ cells/ml in cold serum-free RPMI, added to the cuvettes, mixed, and pulsed once at 300 mV, 975 μF with a Gene Pulser electroporator II (Bio-Rad). After electroporation, the cells were put into four wells of a 24-well plate, each containing 1 ml of RPMI. Cell viability immediately after electroporation was typically around 60%.

Flow Cytometry:

For flow cytometric analysis described in this and subsequent examples, all cells were washed once in FACS buffer (PBS supplemented with 2% FCS and 0.01% sodium azide), resuspended to 200 μl, and stained directly with the appropriate antibodies. The stained cells were washed once, then resuspended in 100 μl FACS buffer containing 5 μg/ml propidium iodide (PI). Unstained and singly stained controls were included in every experiment. Cell data were collected on a FACSCalibur flow cytometer (BD Biosciences, San Jose, Calif.) and four-color analyses (GFP, PE, PI, and allophycocyanin) were done with CellQuest software (BD Biosciences). All data were collected by analyses performed on at least 2.5×10⁵ PI-negative events (viable cells).

The following phycoerythrin (PE) conjugated antibodies were used in this and the following examples: anti-CD4 (clone RM4-5), anti-CD8α (clone 53-6.7), anti-CD25 (clone PC81), anti TCRβ (clone H57-597), anti-CD28 (clone 37.51) and strepavidin. Allophycocyanin (APC)-conjugated anti-CD8α and biotin-conjugated anti-CD3 were also used for analysis. All antibodies were from BD Pharmingen (San Diego, Calif.). All plots shown are gated for viable cells, which were isolated by selecting PI⁻ cells.

Northern Blot Analysis:

For Northern blot analysis, cells were lysed with Trizol reagent (Invitrogen), and total cellular RNA was prepared according to the manufacturer's instructions. CD4/CD8 probe hybridization and was performed as described (11). For the small RNA Northern, total RNA (60 μg) was fractionated on a 10% denaturing polyacrylamide gel and transferred to nylon membrane. The membrane was hybridized to a probe consisting of a 21 nt CD8 siRNA sense strand 5′ end-labeled with ³²P. A 5′ radiolabeled oligonucleotide probe to 5S RNA was used to determine equal loading of RNA. The probe for the siRNA CD8 was taken exactly from the sense strand in the plL3.7 CD8 stem-loop as described in reference 11.

Results

To determine whether lentiviral vectors could deliver shRNAs and silence genes in mammalian cells, the pLL3.7 vector described in Example 2 that carries the U6 RNA polymerase III (polIII) promoter (FIG. 17A) was used. This promoter is known to efficiently transcribe small RNAs that silence gene expression (2, 26). The pLL3.7 vector was also engineered to express EGFP as a reporter gene, permitting infected cells to be tracked by flow cytometry. EGFP expression was driven by a constitutive RNA polymerase II (polII) promoter derived from cytomegalovirus (CMV) (FIG. 17B). This promoter is active in most mammalian tissues (27). The CMV promoter of pLL3.7 was placed between LoxP sites to allow removal of this genetic element if transcript levels from the U6 promoter were not sufficient to silence gene expression due, for example, to possible promoter interference that might decrease expression of shRNAs in infected cells.

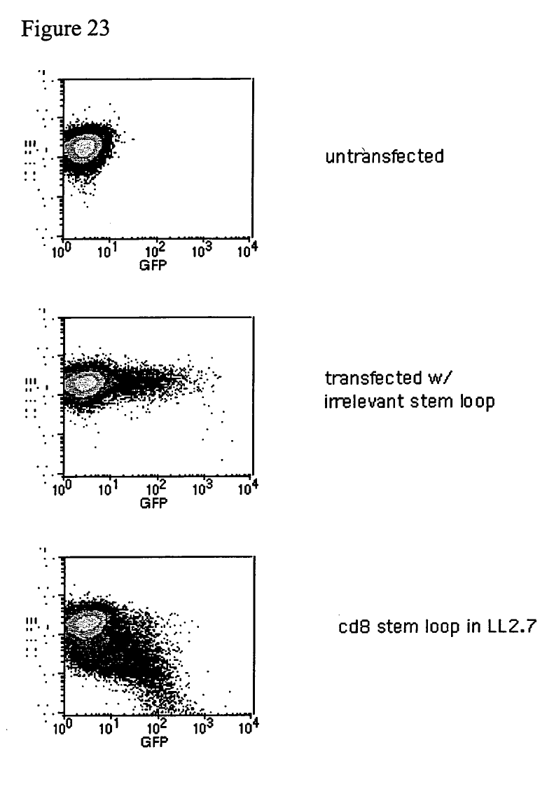

To test whether pLL3.7 could be used to silence gene expression in mammalian cells, the sequence for a shRNA predicted to target the T cell surface molecule, CD8, was introduced into this vector to generate pLL3.7 CD8 (FIG. 17B). The CD8 shRNA duplex sequence (SEQ ID NO: 25: 5′-TGCTACAACTACTACATGAC-3′ when expressed in DNA format) was based on sequences that we had previously characterized in the CD8⁺ E10 thymoma cell line (11, 28), and that we had shown will specifically downregulate CD8 in these cells when introduced as siRNAs. As a first test we electroporated pLL3.7 CD8 or a pLL3.7 vector containing a stem loop targeted to an unrelated sequence (CD25T, a stem loop targeted to CD25 but containing a mutation resulting in an early termination site) into E10 cells, and quantified expression of CD8 in transfected cells by flow cytometry. E10 cells that took up pLL3.7 CD8 or pLL3.7 CD25T DNA could be identified by flow cytometry based on their expression of GFP, i.e., cells that took up the vector became GFP-positive. As shown in FIG. 23, GFP⁺ E10 cells transfected with pLL3.7 CD8 (lower panel) showed on average a 7-fold reduction in CD8 levels relative to cells transfected with pLL3.7 CD25T (middle panel). This result demonstrated that pLL3.7 CD8 was able to silence expression of CD8 in T cells. Since we could detect GFP⁺ and CD8-silenced cells, promoter interference did not present a major barrier for co-expression of shRNAs and a reporter gene.

An aspect of the data that should be noted is that although FIG. 23 appears to suggest that higher levels of GFP correlates with decreased CD8 expression, which would suggest that more copies of the lentiviral DNA result in greater silencing, this is an artifact due to the fact that when subtracting signal to correct for overlap between signals, more signal from the PE (CD8) channel was subtracted from the GFP channel than should have been, which makes the cells with high GFP appear to have less CD8. In reality, it appears that any expression from the U6 promotor, regardless of number of integrants or copies leads to full silencing. A further complication of this experiment is that GFP expression from the plasmid is actually highest after 24 hrs. As a consequence, at 48 hours, there are many silenced cells that have become GFP-.

### EXAMPLE 4

### Production of Infectious Lentiviral Particles using Lentiviral Transfer Plasmids Containing shRNA Sequences

Materials and Methods

Cell Culture and Lentivirus Production.

Cell culture was performed as in Example 3. Lentiviral production was performed as described (24) using packaging plasmids pMDL g/pRRE, pCMV VSV-G, and pRSV-REV, described in references 21 and 40.

Harvesting and titering lentivirus.

Lentivirus was harvested and titered according to the following protocol:

Harvesting:

1. Harvest supernatant from cells and spin at 25,000 rpm for 1.5 hrs
2. Remove all liquid, add x volume of PBS (between 15 and 200 ul), and allow to sit overnight at 4 degrees
3. Pipette up and down 20 times
4. Use or aliquot and flash-freeze in liquid N2, store at −80.
   Titering:
1. Plate 4×10⁵ 293.T cells/well in a 6-well plate 12-24 hours prior to titering. It is helpful to have an additional well as a negative control that you mock infect with D10+polybrene but without virus.
2. Make a stock solution of D10×medium with 8 μg/ml polybrene.
3. Generate a 10-fold dilution series of virus in the D10+polybrene. Using 1.5 mls/well you should have 1 μl, 0.1, 0.01, 0.001, 0.0001, and 0.0000 μL of virus/well.
4. Incubate at 37 degrees O/N. Replace media with fresh D10.
5. At least 48 hours after infection trypsinize cells for FACS analysis. (Trypsinize, inactivate with media, spin, and resuspend in cold PBS).
6. FACS analyze for EGFP expression and record the percentage of cells that are EGFP positive.
7. Use a well that has between 0.1% and 10% of cells expressing EGFP to determine titer. Sample calculation assuming 1% infection from the well with 0.01 μl of virus: 0.01 (percentage of cells that are EGFP positive)×4×10⁵=4×10³ positive cells. 4×10³×100 (dilution factor)=4×10⁵ viral particles/ul.

In general at least 5×10⁵ viral particles/ul should be used for embryo infections.

Transfection of293 cells. The following protocol was used for transfection of 293 cells in this and the following examples.
1. Plate 12×10⁶ 293.T cells in 20 ml on a 15 cm² plate 24 hours before transfection. In general, two 15 cm plates per virus are used. It is highly preferred that the cells be well-maintained and of relatively low passage number.
2. Mix the following DNAs (preferably made using Endo-free Qiagen Kits according to the manufacturer's instructions) in a FACS tube. The DNAs should be in Endo-free TE at a concentration of 0.5 μg/μl.
   For 3 plasmid system:
      20 μg vector (transfer plasmid, e.g., pLL3.7 CD8)
      10 μg pVSVG (envelope plasmid)
      15 μg Δ8.9 (packaging plasmid)
   For 4 plasmid, system (recommended),
      20 μg vector, (transfer plasmid, e.g., pLL3.7 CD8)
      10 μg pVSVG (envelope plasmid)
      10 μg RSV-REV (plasmid supplies Rev protein)
      10 μg pMDL g/p RRE (packaging plasmid)

The envelope plasmid, packaging plasmids, and Rev-supplying plasmid are described in further detail in references 21, 24, 40, and 41.

Add 400 μl 1.25 M CaCl₂ and 1.5 ml H₂0 and mix by tapping gently.

The following steps are done 1 plate at a time.
3. Add 2 ml of 2×HBS dropwise to DNA mixture while bubbling with a Pasteur pipette. When finished, continue to bubble for 12-15 seconds.
4. Take plate of 293T out of the incubator (plate remains in incubator for long as possible), and add transfection mixture dropwise all over the plate. Gently swirl plate from front to back, and return immediately to incubator.
5. 3.5 to 4 hours later, remove media, wash 2× with 10 ml warm PBS, and add 20 ml warm D10 onto plate and place in incubator.
6. 36-48 hours after transfection, harvest viral supernatant and spin at 2000 rpm, 7 min at 4° C. in a 50 ml tube.
7. Filter viral SN through 0.45 um filter. Add 35 ml of filtered supernatant to an ultracentrifuge tube. Balance tubes with additional media. Cover tubes with small piece of parafilm. (It is useful to titer some of the leftover supernatant to determine if there is loss of virus during concentration.)
8. Spin tubes using a SW-28 rotor at 25,000 rpm, 90 min, 4° C. Decant liquid and leave tube upside down on kimwipe for 10 min. Aspirate remaining media being careful not to touch bottom of tube.
9. Add 15 μl cold PBS (for embryo infections, or any volume you wish) and leave tube at 4° C. O/N with no shaking.
10. To resuspend, hold tube at angle and pipet fluid over pellet 20 times, being careful not to touch pellet with tip. It is expected that the pellet not be resuspended after this is complete. This pellet does not contain virus and can be discarded.
11. Aliquot or use virus. Virus should be aliquoted, flash-frozen in liquid nitrogen and stored at −80. There should be no change in titer with freezing concentrated virus. Avoid multiple freeze-thaws.

Results

To test whether pLL3.7 vectors could be used to generate infectious lentivirus particles, 293T cells were transfected with pLL3.7 or pLL3.7 CD8, and three lentiviral packaging plasmids developed by Miyoshi et al. (29). Our initial concern was that a lentiviral construct containing shRNA sequences would itself be susceptible to RNA interference, thus preventing generation of viral (RNA) genomes and production of infectious viral particles. We failed to find any evidence of this type of auto-inhibition. We were able to generate viral stocks of pLL3.7 and pLL3.7 CD8 that could infect mouse fibroblast 3T3 cells, as gauged by GFP expression, and the titre of these viral stocks was always qualitatively similar. These results demonstrated that lentiviruses capable of mediating gene silencing can be generated efficiently. In other words, shRNAs generated by lentiviral vectors can target endogenous cellular transcripts but do not inhibit production of viral RNAs carrying the same sequences.

### EXAMPLE 5

### Stable Silencing of Genes and Production of Processed shRNAs in T Cells by a Lentiviral Vector

Materials and Methods

Cell Culture, Lentivirus Production, and Lentivirus Infection.

Cell culture was performed as in Example 2. Lentiviral production and infection were performed as described (24) in this and following examples unless otherwise indicated. For some experiments, sorted populations of infected E10 cells were maintained in long-term culture. E10 cells pLL3.7 CD8 (CD8 RNAi virus) were sorted four days after infection for GFP expression and low CD8 expression, while cells infected with control virus were sorted for GFP expression only. Each population was cultured for 1 month and analyzed for CD8 expression via flow cytometry at weekly intervals.

Results

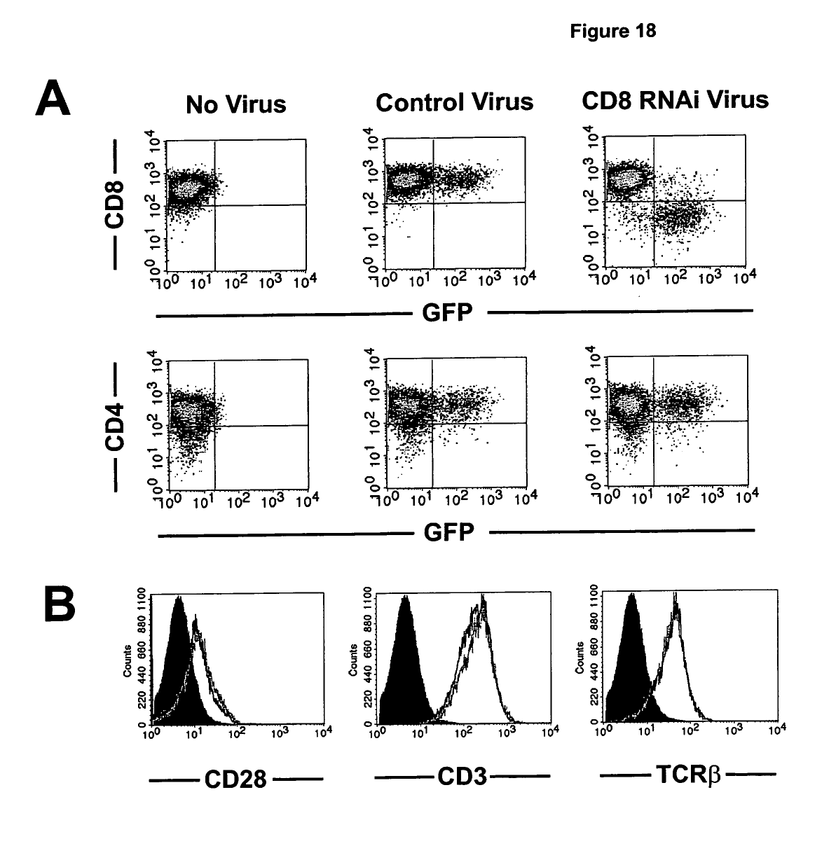

We examined whether the LentiLox system could be used to silence gene expression upon infection of mammalian cells. To accomplish this, E10 cells were infected with pLL3.7 and pLL3.7 CD8 viruses. A low viral titre was used so that only a fraction of cells became infected, as gauged by GFP expression (FIG. 18A). This allowed us to follow the fate of both infected and non-infected cells simultaneously. Infected (GFP⁺) cells on average showed a 16-fold reduction of CD8 expression. FIG. 2a shows density plots indicating the expression levels of CD4 and CD8 48 hours post-infection.

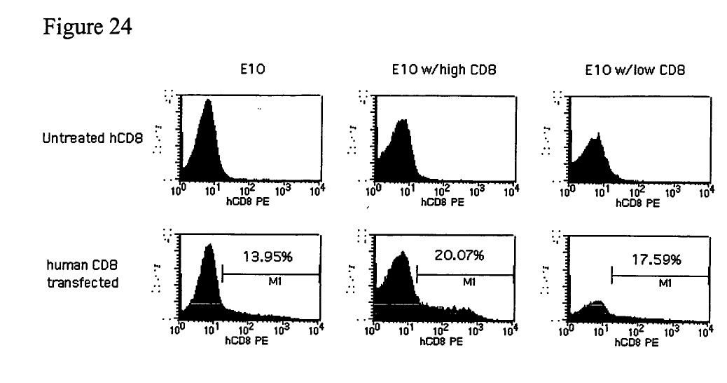

Expression of CD8 and of other surface proteins was measured at 48 hours and at various times thereafter. Inhibition of CD8 expression in E10 cells infected with pLL3.7 CD8 was specific since levels of other surface proteins were not altered (FIG. 18B). Furthermore, in a subline of E10 cells engineered to express human CD8, which differs from mouse CD8 by 4 out of 19 nucleotides in the region that we targeted, we showed that pLL3.7 CD8 selectively reduced expression of the mouse protein. FIG. 24 shows expression of human CD8 in 3 populations of cells that either were (lower panels) or were not (upper panels) transfected with a construct encoding human CD8 (hCD8). The leftmost panels show expression of human CD8 in wild type ES cells, illustrating expression of hCD8 in transfected cells (lower left panel; cells below bar display hCD8 expression. The middle panels show expression of human CD8 in a population of ES cells that were infected with pLL3.7 CD8 and displayed effective silencing of mouse CD8 (low CD8). As shown in the lower middle panel, this population of cells did not display silencing of human CD8. The rightmost panels show expression of human CD8 in a population of ES cells that were infected with pLL3.7 CD8 and did not display extensive silencing of mouse CD8 (high CD8). As shown in the lower right panel, this population of cells also did not display silencing of human CD8. These data show that an shRNA targeted to mouse CD8 does not silence human CD8, confirming the specificity of silencing. Cells infected with a control virus (pLL3.7) or a virus that expressed a neuron-specific shRNA (pLL3.7 Mena+) showed no decrease in CD8 levels (FIG. 18A and data not shown).

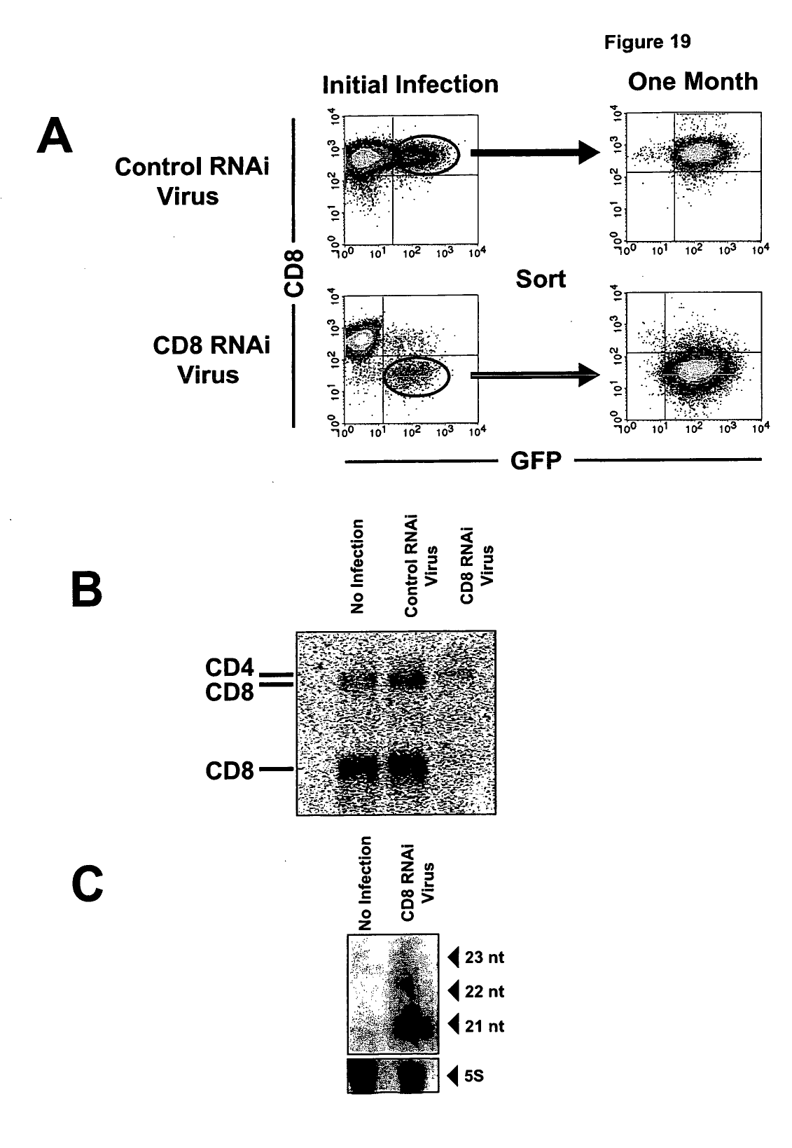

To confirm that the decrease in surface CD8 expression seen in E10 cells resulted from mRNA degradation, we assayed CD8 mRNA levels in sorted (GFP+) cell populations infected either with a control virus (pLL3.7) or CD8 RNAi virus (pLL3.7 CD8) (FIG. 19A). Consistent with results showing significant reduction of CD8 protein levels, the amount of CD8 transcripts in E10 cells infected with pLL3.7 CD8 was 13-fold lower then in controls (FIG. 19B). The same cells expressed normal amounts of CD4 transcripts (FIG. 19B), confirming the specificity of the RNAi knockdown. We also examined whether the shRNAs encoded by pLL3.7 CD8 were processed into the approximately 21 nucleotide-long RNAs reported to mediate RNAi (12) by blotting cellular RNA extracts with a probe directed against the anti-sense strand of the CD8 stem loop. Only the pLL3.7 CD8 infected cells produced CD8 siRNAs (FIG. 19C). The dominant population of siRNAs detected was 21 bases in length; although 1-2 bp longer species of siRNAs were also present (FIG. 19C). No precursor shRNA was visible on the autoradiogram.

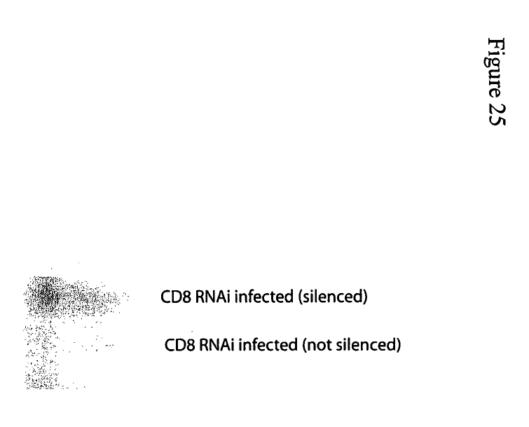

to test the stability of lentivirus-mediated RNAi in mammalian cells, we followed expression of CD8 in long-term cultures of E10 cells infected with pLL3.7 or pLL3.7 CD8. Cells were sorted based on GFP and CD8 expression levels four days after infection with lentivirus, and subsequently monitored for expression of these proteins weekly. No change in expression of CD8 was observed and these cells remained uniformly GFP positive, demonstrating that RNAi mediated by the integrated lentivirus was stable (FIG. 19A). In each experiment a small fraction (2 to 15%) of E10 cells infected with pLL3.7 CD8 showed no evidence of gene silencing, maintaining wild type CD8 expression (FIG. 19A). This was not necessarily the result of a low copy number of integrated viruses or poor expression of viral genes since some of these cells expressed very high levels of GFP (FIG. 19A). As shown in the Northern blot in FIG. 25, we were able to determine that these cells expressed little, if any shRNAs directed against CD8, suggesting that the activity of the polIII promoter was reduced.

### EXAMPLE 6

### Functional Gene Silencing in Differentiated Mammalian Cells Induced by Lentiviruses

Materials and Methods

T-Cell Purification and Stimulation.

Cells were harvested from spleen and lymph nodes. They were plated in RPMI with 10% FBS supplemented with 1 ug/ml ova peptide. Cells were infected 24 and 48 hours after plating and analyzed 72 hours after plating. This activation method yields >90% purity of T-cells.

Viral Infection.

Spin infection was performed as described for retrovirus in van Parijs, L., et al., Immunity, 11:281, 1999 using 50 ul of concentrated lentivirus.

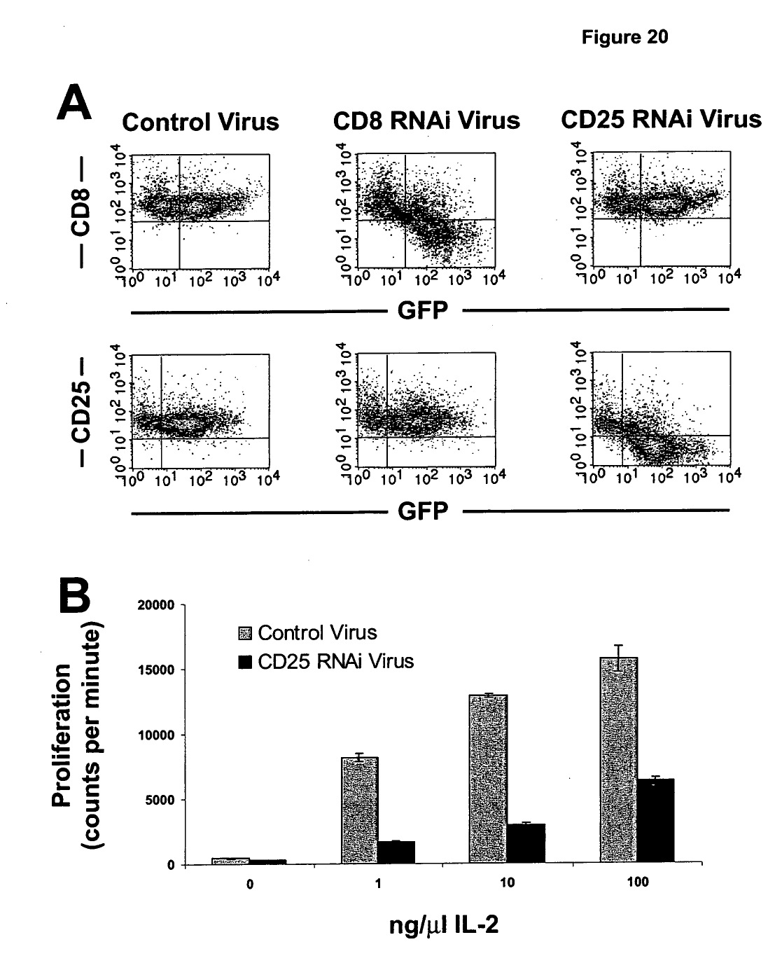

We tested whether the LentiLox-based RNAi system could be used to silence gene expression in primary mammalian cells. In these experiments, we purified CD8⁺ T cells from the spleens of OTI T-cell receptor (TCR) transgenic mice, activated them with cognate peptide antigen, and then infected these cells with pLL3.7 or pLL3.7 CD8. After three days in culture, the T cells were harvested and analysed for GFP and CD8 expression by flow cytometry. Between 68 and 82% of the cells were infected, as gauged by GFP expression (FIG. 4a). The infected (GFP⁺) population reproducibly showed approximately a 14-fold reduction in CD8 expression, demonstrating that lentivirus-driven expression of shRNAs efficiently silenced gene expression in primary T cells (FIG. 20A). This effect of pLL3.7 CD8 was specific since infected cells showed normal expression of other T cell surface markers (FIG. 20A).

We next examined whether the degree of gene silencing achieved in primary mammalian cells using the LentiLox system was functionally relevant. To accomplish this we performed an experiment aimed at studying the biological effects of targeting the IL-2 receptor (IL-2R) in T cells using lentivirus-mediated RNAi. IL-2 is an important growth factor for T cells, and T cells derived from mice that lack the receptor for this cytokine fail to proliferate in vitro (31). To determine whether we could phenocopy IL-2R-deficiency in primary T cells using lentivirus-mediated RNAi, we designed a shRNA against the alpha chain of the IL-2 receptor (CD25) and used this sequence to create pLL3.7 CD25. The shRNA sequences were as follows:

CD25 sense: 5′-tgcattcacctaatcggctgttcaagagacagccgattaggtgaatgcttttttg-3′ (SEQ ID NO: 38)

CD25 antisense: 5′-gtcaccaaaaaagcattcacctaatcggctgtctcttgaacagccgattaggtgaatgca-3′ (SEQ ID NO: 39)

In most experiments between 70 and 85% of activated CD8+TCR transgenic T cells were infected with this virus (FIG. 20A). Infected cells on average showed a 25-fold reduction in IL-2Rα chain expression, but expressed normal levels of other surface markers (FIG. 20A). These cells were challenged with increasing concentrations of IL-2, resulting in a 4- to 5-fold reduction in the response to this cytokine (FIG. 20B). Therefore, the LentiLox RNAi system can be used to phenocopy loss-of-function in primary T cells.

### EXAMPLE 7

### Functional Silencing of Genes in Embryonic Stem Cell-Derived Mice by a Lentiviral Vector

Materials and Methods

Generation of Lentiviral Transfer Plasmids Containing shRNAs Targeted to Mena+, Beta-Catenin, and p53.

Oligonucleotides having the following sequences were inserted into pLL3.7 as described above to produce lentiviral transfer plasmids capable of directing expression of shRNAs targeted to Mena+, Beta-catenin, or p53 transcripts.

Mena+sense: 5′-tgtcctgtgcctggcctactttcaagagaagtaggccaggcacaggactttttggaaac-3′ (SEQ ID NO: 26)

| | |
|---|---|
| Mena + antisense: | |
| 5′-tcgagttcccaaaaagtcctgtgcctggcctac | (SEQ ID NO:27) |
| ttctcttgaaagtaggccaggcacaggaca-3′ | |
| Beta-catenin sense: | |
| 5′-tgtccagcgcttggctgaacttcaagagtgttc | (SEQ ID NO:28) |
| agccaagcgctggactttttggaaa-3′ | |
| Beta antisense: | |
| 5′-tcgatttccaaaaagtccagcgcttggctgaac | (SEQ ID NO:29) |
| actcttgaagttcagccaagcgctggaca-3′ | |
| P53 sense: | |
| 5′-tggtctaagtggagcccttcgagtgttagaagc | (SEQ ID NO:30) |
| ttgtgacactcggagggcttcacttgggccttt | |
| ttggaaa-3′ | |
| P53 antisense: | |
| 5′-tcgatttccaaaaaggcccaagtgaagccctcc | (SEQ ID NO:31) |
| gagtgtcacaagcttctaacactcgaagggctccac | |
| ttgacca-3′ | |

ES Cells:

AK7 ES cells were maintained and infected as described (23). Clones of ES cells were picked, expanded, and analyzed by flow cytometry for GFP expression. If the clone contained a mixed population of infected and uninfected cells, the GFP population was purified by fluorescence activated cell sorting.

Production of Transgenic Mice:

Transgenic mice were generated essentially as described in reference 24.

Results

A unique feature of lentivirus-based vectors is that they can stably express transgenes in stem cells and are not silenced during development, allowing for the efficient generation of transgenic mice (23, 24). We tested whether the LentiLox RNAi system could be used to silence gene expression in stem cells, as well as animals generated from these cells. To accomplish this we infected embryonic stem cells with versions of the pLL3.7 vector that expressed shRNAs against CD8 (pLL3.7 CD8), Mena+(pLL3.7 Mena+), or p53 (pLL3.7 p53).

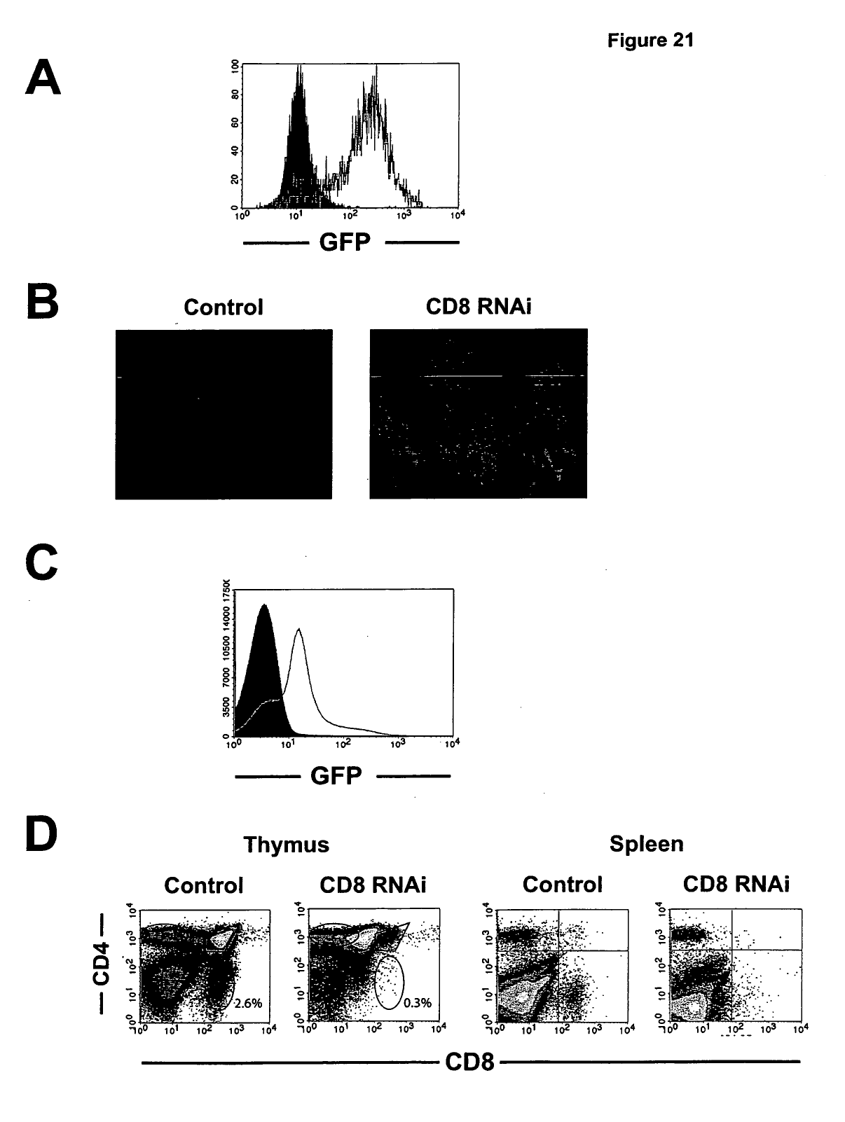

We found that these vectors could efficiently infect embryonic stem cells, and we are able to generate and maintain stable lines of infected ES cells (FIG. 21A, and data not shown).

To test whether ES cells infected with RNAi lentivirus were capable of giving rise to progeny that showed gene silencing, we generated uniformly GFP+ES cell populations infected with pLL3.7 CD8, pLL3.7 Mena+, or the empty vector, pLL3.7, by cell sorting. Ten to twelve of these cells were injected into day 3 blastocysts, which were subsequently implanted into pseudopregnant recipients. To ensure that the lentivirus-infected ES cells contributed to immune tissues in the chimeric offspring, we used RAG2^{-/-} blastocysts in these experiments. This genetic lesion blocks the development of B and T cells, so that any immune cells present in the chimeric progeny must be derived from the injected (wild type) ES cells (32). Using this approach we generated mice derived from ES cells infected with pLL3.7 CD8, pLL3.7 Mena+, and pLL3.7. The degree of chimerism in these animals was between 50 and 90% as gauged by GFP fluorescence analysis of whole mice, as well as dissected organs (FIG. 21B and data not shown). This result demonstrated that cells expressing siRNAs were not selected against during development and that these cells were able to contribute to all tissues in the body.

In our chimeric mice, almost all cells in the lymphoid organs expressed GFP, indicating that they were derived from the injected ES cells (FIG. 21C and data not shown). To examine whether lentivirus-mediated expression of shRNAs resulted in the silencing of CD8 in vivo, we harvested the thymus and spleen of 7 day-old chimeric mice and stained the cells present in these organs with antibodies against CD8 and CD4 according to standard techniques. We found that developing T cells in the thymus of pLL3.7 CD8 mice showed a 7-fold reduction in CD8 expression (FIG. 21D). Furthermore, no mature CD8+ T cells were detected in this organ or in the spleen (FIG. 21D). In contrast, thymocytes from these mice showed normal expression of CD4 and normal numbers of mature CD4+T cells were found in their lymphoid organs. (FIG. 21D). T cell differentiation and numbers were normal in mice derived from pLL3.7 Mena+ and pLL3.7 infected ES cells (FIG. 21D and data not shown).

### EXAMPLE 8

### Cre-Mediated Extinguishing of a Transgene Expressed from a Lentiviral Vector

This example demonstrates that introduction of Cre recombinase into cells expressing a transgene from a lentiviral vector of the invention extinguishes expression of the transgene.

Materials and Methods

Expression of EGFP Using a Lentiviral Vector.

A 50% confluent 10 cm plate of D7 cells (See Bear J E, et al., Cell 2000 Jun. 23;101(7):717-2 for description of cells and culture conditions.), was infected with 100 ul of concentrated pLL3.7 B-catenin lentivirus, which expressed GFP as a transgene between two LoxP sites. Infected cells (pLL3.7 B-catenin containing cells) were sorted based upon expression of EGFP.

Introduction of Cre.

A 50% confluent 6 cm plate of sorted D7 pLL3.7 B-catenin containing cells was infected with adenovirus expressing the Cre recombinase (Jackson EL, et al., Genes Dev 2001 Dec. 15;15(24):3243-8×10⁵ infectious units were used in the infection. Cells were expanded for 10 days to allow time for expression of Cre protein, deletion of the loxP-CMVegfp-loxP cassette, and depletion of EGFP protein pools. Cells were then analyzed by flow cytometry for expression of EGFP. Cells were also sorted based upon loss of EGFP expression and expanded.

Results

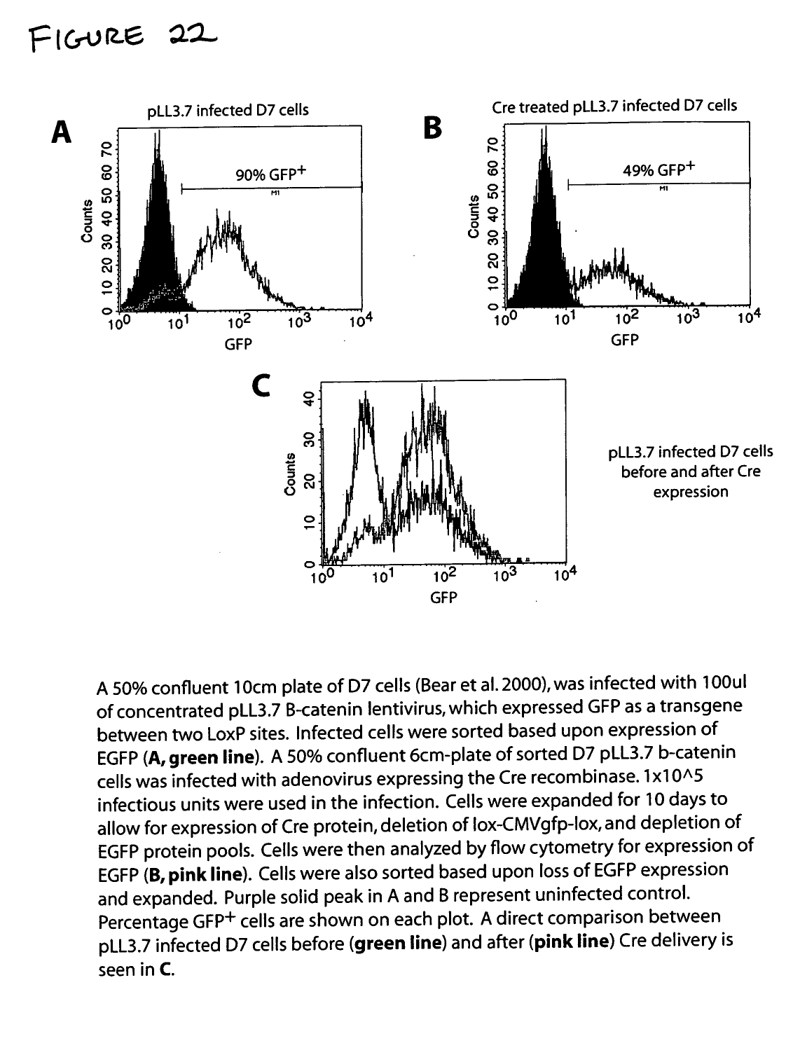

FIG. 22A shows flow cytometric analysis of EGFP expression in cells infected with an EGFP-expressing lentiviral vector in which the promoter and EGFP coding sequences are floxed. Flow cytometry was performed at least 48 hours after infection. The solid purple peaks in FIG. 22A and 22B represent uninfected cells. As shown in FIG. 22A, ninety percent of the infected cells express EGFP. The population of cells expressing EGFP is shown with a green line. FIG. 22B shows flow cytometric analysis of EGFP expression in cells infected with an EGFP-expressing lentiviral vector 10 days after induction of Cre expression. The percentage of EGFP-expressing cells is reduced to 49%. FIG. 22C shows a direct comparison between pLL3.7 infected D7 cells before (green line) and after (pink line) Cre delivery. Induction of Cre extinguished expression of the floxed transgene in approximately half the cells. (The adenoviral titer was not high enough to infect all cells, thus cells in which the transgene was not extinguished were probably not infected with adenovirus.)

Sequences of pBFGW and pLL3.0-pLL3.7.

This section presents the sequences of plasmids pBFGW and pLL3.0-pLL3.7 in the form of GenBank files.

pBFGW

| LOCUS PBFGW.GB | | 10441 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV 1” |
| | gene | 9448..10308 |
| | | /note=“AmpR” |
| | rep_origin | 8630..9303 |
| | | /note=“pUC” |
| | polyA_signal | 6452..6666 |
| | | /note=“BGH pA” |
| | gene | 7613..7987 |
| | | /note=“ZeoR” |
| | polyA_signal | 8117..8246 |
| | | /note=“5V40 pA” |
| | rep_origin | 6729..7142 |
| | | /note=“f1 origin” |
| | promoter | 7205..7530 |
| | | /note=“SV40 ori” |
| | LTR | 835..1509 |
| | | /note=“5′ LRT” |
| | misc_feature | 1533..2390 |
| | | /note=“psi sequence” |
| | misc_feature | 2416..2593 |
| | | /note=“FLAP” |
| | promoter | 2612..2974 |
| | | /note=“CMV 2” |
| | promoter | 2798..4229 |
| | | /note=“Beta actin promoter” |
| | intron | 4234..4327 |
| | | /note=“beta globin intron” |
| | gene | 4373..5089 |
| | | /note=“EGFP” |
| | misc_feature | 5132..5721 |
| | | /note=“WRE” |
| | misc_feature | 5737..6426 |
| | | /note=“3′ LTR” |

| | |
|---|---|
| BASE COUNT 2414 A | 2697 C 2911 G 2419 T 0 OTHER |
| ORIGIN - | |

| (SEQ ID NO:1) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG CTGATCTTCA GACCTGGAGG AGGAGATATG |
| | 1561 AGGGACAATT GGAGAAGTGA ATTATATAAA TATAAAGTAG TAAAAATTGA ACCATTAGGA |
| | 1621 GTAGCACCCA CCAAGGCAAA GAGAAGAGTG GTGCAGAGAG AAAAAAGAGC AGTGGGAATA |
| | 1681 GGAGCTTTGT TCCTTGGGTT CTTGGGAGCA GCAGGAAGCA CTATGGGCGC AGCGTCAATG |
| | 1741 ACGCTGACGG TACAGGCCAG ACAATTATTG TCTGGTATAG TGCAGCAGCA GAACAATTTG |
| | 1801 CTGAGGGCTA TTGAGGCGCA ACAGCATCTG TTGCAACTCA CAGTCTGGGG CATCAAGCAG |
| | 1861 CTCCAGGCAA GAATCCTGGC TGTGGAAAGA TACCTAAAGG ATCAACAGCT CCTGGGGATT |
| | 1921 TGGGGTTGCT CTGGAAAACT CATTTGCACC ACTGCTGTGC CTTGGAATGC TAGTTGGAGT |
| | 1981 AATAAATCTC TGGAACAGAT TTGGAATCAC ACGACCTGGA TGGAGTGGGA CAGAGAAATT |
| | 2041 AACAATTACA CAAGCTTAAT ACACTCCTTA ATTGAAGAAT CGCAAAACCA GCAAGAAAAG |
| | 2101 AATGAACAAG AATTATTGGA ATTAGATAAA TGGGCAAGTT TGTGGAATTG GTTTAACATA |
| | 2161 ACAAATTGGC TGTGGTATAT AAAATTATTC ATAATGATAG TAGGAGGCTT GGTAGGTTTA |
| | 2221 AGAATAGTTT TTGCTGTACT TTCTATAGTG AATAGAGTTA GGCAGGGATA TTCACCATTA |
| | 2281 TCGTTTCAGA CCCACCTCCC AACCCCGAGG GGACCCGACA GGCCCGAAGG AATAGAAGAA |
| | 2341 GAAGGTGGAG AGAGAGACAG AGACAGATCC ATTCGATTAG TGAACGGATC GGCACTGCGT |
| | 2401 GCGCCAATTC TGCAGACAAA TGGCAGTATT CATCCACAAT TTTAAAAGAA AAGGGGGGAT |
| | 2461 TGGGGGGTAC AGTGCAGGGG AAAGAATAGT AGACATAATA GCAACAGACA TACAAACTAA |
| | 2521 AGAATTACAA AAACAAATTA CAAAAATTCA AAATTTTCGG GTTTATTACA GGGACAGCAG |
| | 2581 AGATCCAGTT TGGTTAATTA ACTGCAGGAA TCTAGTTATT AATAGTAATC AATTACGGGG |
| | 2641 TCATTAGTTC ATAGCCCATA TATGGAGTTC CGCGTTACAT AACTTACGGT AAATGGCCCG |
| | 2701 CCTGGCTGAC CGCCCAACGA CCCCCGCCCA TTGACGTCAA TAATGACGTA TGTTCCCATA |
| | 2761 GTAACGCCAA TAGGGACTTT CCATTGACGT CAATGGGTGG AGTATTTACG GTAAACTGCC |
| | 2821 CACTTGGCAG TACATCAAGT GTATCATATG CCAAGTACGC CCCCTATTGA CGTCAATGAC |
| | 2881 GGTAAATGGC CCGCCTGGCA TTATGCCCAG TACATGACCT TATGGGACTT TCCTACTTGG |
| | 2941 CAGTACATCT ACGTATTAGT CATCGCTATT ACCATGGTCG AGGTGAGCCC CACGTTCTGC |
| | 3001 TTCACTCTCC CCATCTCCCC CCCCTCCCCA CCCCCAATTT TGTATTTATT TATTTTTTAA |
| | 3061 TTATTTTGTG CAGCGATGGG GGCGGGGGGG GGGGGGGGGC GCGCGCCAGG CGGGGCGGGG |
| | 3121 CGGGGCGAGG GGCGGGGCGG GGCGAGGCGG AGAGGTGCGG CGGCAGCCAA TCAGAGCGGC |
| | 3181 GCGCTCCGAA AGTTTCCTTT TATGGCGAGG CGGCGGCGGC GGCGGCCCTA TAAAAAGCGA |
| | 3241 AGCGCGCGGC GGGCGGGGAG TCGCTGCGAC GCTGCCTTCG CCCCGTGCCC CGCTCCGCCG |
| | 3301 CCGCCTCGCG CCGCCCGCCC CGGCTCTGAC TGACCGCGTT ACTCCCACAG GTGAGCGGGC |
| | 3361 GGGACGGCCC TTCTCCTCCG GGCTGTAATT AGCGCTTGGT TTAATGACGG CTTGTTTCTT |
| | 3421 TTCTGTGGCT GCGTGAAAGC CTTGAGGGGC TCCGGGAGGG CCCTTTGTGC GGGGGGAGCG |
| | 3481 GCTCGGGGGG TGCGTGCGTG TGTGTGTGCG TGGGGAGCGC CGCGTGCGGC TCCGCGCTGC |
| | 3541 CCGGCGGCTG TGAGCGCTGC GGGCGCGGCG CGGGGCTTTG TGCGCTCCGC AGTGTGCGCG |
| | 3601 AGGGGAGCGC GGCCGGGGGC GGTGCCCCGC GGTGCGGGGG GGGCTGCGAG GGGAACAAAG |
| | 3661 GCTGCGTGCG GGGTGTGTGC GTGGGGGGGT GAGCAGGGGG TGTGGGCGCG TCGGTCGGGC |
| | 3721 TGCAACCCCC CCTGCACCCC CCTCCCCGAG TTGCTGAGCA CGGCCCGGCT TCGGGTGCGG |
| | 3781 GGCTCCGTAC GGGGCGTGGC GCGGGGCTCG CCGTGCCGGG CGGGGGGTGG CGGCAGGTGG |
| | 3841 GGGTGCCGGG CGGGGCGGGG CCGCCTCGGG CCGGGGAGGG CTCGGGGGAG GGGCGCGGCG |
| | 3901 GCCCCCGGAG CGCCGGCGGC TGTCGAGGCG CGGCGAGCCG CAGCCATTGC CTTTTATGGT |
| | 3961 AATCGTGCGA GAGGGCGCAG GGACTTCCTT TGTCCCAAAT CTGTGCGGAG CCGAAATCTG |
| | 4021 GGAGGCGCCG CCGCACCCCC TCTAGCGGGC GCGGGGCGAA GCGGTGCGGC GCCGGCAGGA |
| | 4081 AGGAAATGGG CGGGGAGGGC CTTCGTGCGT CGCCGCGCCG CCGTCCCCTT CTCCCTCTCC |
| | 4141 AGCCTCGGGG CTGTCCGCGG GGGGACGGCT GCCTTCGGGG GGGACGGGGC AGGGCGGGGT |
| | 4201 TCGGCTTCTG GCGTGTGACC GGCGGCTCTA GAGCCTCTGC TAACCATGTT CATGCCTTCT |
| | 4261 TCTTTTTCCT ACAGCTCCTG GGCAACGTGC TGGTTATTGT GCTGTCTCAT CATTTTGGCA |
| | 4321 AAGAATTGAT TTGATACCGC GGGCCCGGGA TCCCCGGGTA CCGGTCGCCA CCATGGTGAG |
| | 4381 CAAGGGCGAG GAGCTGTTCA CCGGGGTGGT GCCCATCCTG GTCGAGCTGG ACGGCGACGT |
| | 4441 AAACGGCCAC AAGTTCAGCG TGTCCGGCGA GGGCGAGGGC GATGCCACCT ACGGCAAGCT |
| | 4501 GACCCTGAAG TTCATCTGCA CCACCGGCAA GCTGCCCGTG CCCTGGCCCA CCCTCGTGAC |
| | 4561 CACCCTGACC TACGGCGTGC AGTGCTTCAG CCGCTACCCC GACCACATGA AGCAGCACGA |
| | 4621 CTTCTTCAAG TCCGCCATGC CCGAAGGCTA CGTCCAGGAG CGCACCATCT TCTTCAAGGA |
| | 4681 CGACGGCAAC TACAAGACCC GCGCCGAGGT GAAGTTCGAG GGCGACACCC TGGTGAACCG |
| | 4741 CATCGAGCTG AAGGGCATCG ACTTCAAGGA GGACGGCAAC ATCCTGGGGC ACAAGCTGGA |
| | 4801 GTACAACTAC AACAGCCACA ACGTCTATAT CATGGCCGAC AAGCAGAAGA ACGGCATCAA |
| | 4861 GGTGAACTTC AAGATCCGCC ACAACATCGA GGACGGCAGC GTGCAGCTCG CCGACCACTA |
| | 4921 CCAGCAGAAC ACCCCCATCG GCGACGGCCC CGTGCTGCTG CCCGACAACC ACTACCTGAG |
| | 4981 CACCCAGTCC GCCCTGAGCA AAGACCCCAA CGAGAAGCGC GATCACATGG TCCTGCTGGA |
| | 5041 GTTCGTGACC GCCGCCGGGA TCACTCTCGG CATGGACGAG CTGTACAAGT AAAGCGGCCG |
| | 5101 CGACTCTAGA ATTCGATATC AAGCTTATCG ATAATCAACC TCTGGATTAC AAAATTTGTG |
| | 5161 AAAGATTGAC TGGTATTCTT AACTATGTTG CTCCTTTTAC GCTATGTGGA TACGCTGCTT |
| | 5221 TAATGCCTTT GTATCATGCT ATTGCTTCCC GTATGGCTTT CATTTTCTCC TCCTTGTATA |
| | 5281 AATCCTGGTT GCTGTCTCTT TATGAGGAGT TGTGGCCCGT TGTCAGGCAA CGTGGCGTGG |
| | 5341 TGTGCACTGT GTTTGCTGAC GCAACCCCCA CTGGTTGGGG CATTGCCACC ACCTGTCAGC |
| | 5401 TCCTTTCCGG GACTTTCGCT TTCCCCCTCC CTATTGCCAC GGCGGAACTC ATCGCCGCCT |
| | 5461 GCCTTGCCCG CTGCTGGACA GGGGCTCGGC TGTTGGGCAC TGACAATTCC GTGGTGTTGT |
| | 5521 CGGGGAAATC ATCGTCCTTT CCTTGGCTGC TCGCCTGTGT TGCCACCTGG ATTCTGCGCG |
| | 5581 GGACGTCCTT CTGCTACGTC CCTTCGGCCC TCAATCCAGC GGACCTTCCT TCCCGCGGCC |
| | 5641 TGCTGCCGGC TCTGCGGCCT CTTCCGCGTC TTCGCCTTCG CCCTCAGACG AGTCGGATCT |
| | 5701 CCCTTTGGGC CGCCTCCCCG CATCGATACC GTCGACCTCG AGACCTAGAA AAACATGGAG |
| | 5761 CAATCACAAG TAGCAATACA GCAGCTACCA ATGCTGATTG TGCCTGGCTA GAAGCACAAG |
| | 5821 AGGAGGAGGA GGTGGGTTTT CCAGTCACAC CTCAGGTACC TTTAAGACCA ATGACTTACA |
| | 5881 AGGCAGCTGT AGATCTTAGC CACTTTTTAA AAGAAAAGGG GGGACTGGAA GGGCTAATTC |
| | 5941 ACTCCCAACG AAGACAAGAT ATCCTTGATC TGTGGATCTA CCACACACAA GGCTACTTCC |
| | 6001 CTGATTGGCA GAACTACACA CCAGGGCCAG GGATCAGATA TCCACTGACC TTTGGATGGT |
| | 6061 GCTACAAGCT AGTACCAGTT GAGCAAGAGA AGGTAGAAGA AGCCAATGAA GGAGAGAACA |
| | 6121 CCCGCTTGTT ACACCCTGTG AGCCTGCATG GGATGGATGA CCCGGAGAGA GAAGTATTAG |
| | 6181 AGTGGAGGTT TGACAGCCGC CTAGCATTTC ATCACATGGC CCGAGAGCTG CATCCGGACT |
| | 6241 GTACTGGGTC TCTCTGGTTA GACCAGATCT GAGCCTGGGA GCTCTCTGGC TAACTAGGGA |
| | 6301 ACCCACTGCT TAAGCCTCAA TAAAGCTTGC CTTGAGTGCT TCAAGTAGTG TGTGCCCGTC |
| | 6361 TGTTGTGTGA CTCTGGTAAC TAGAGATCCC TCAGACCCTT TTAGTCAGTG TGGAAAATCT |
| | 6421 CTAGCAGGGC CCGTTTAAAC CCGCTGATCA GCCTCGACTG TGCCTTCTAG TTGCCAGCCA |
| | 6481 TCTGTTGTTT GCCCCTCCCC CGTGCCTTCC TTGACCCTGG AAGGTGCCAC TCCCACTGTC |
| | 6541 CTTTCCTAAT AAAATGAGGA AATTGCATCG CATTGTCTGA GTAGGTGTCA TTCTATTCTG |
| | 6601 GGGGGTGGGG TGGGGCAGGA CAGCAAGGGG GAGGATTGGG AAGACAATAG CAGGCATGCT |
| | 6661 GGGGATGCGG TGGGCTCTAT GGCTTCTGAG GCGGAAAGAA CCAGCTGGGG CTCTAGGGGG |
| | 6721 TATCCCCACG CGCCCTGTAG CGGCGCATTA AGCGCGGCGG GTGTGGTGGT TACGCGCAGC |
| | 6781 GTGACCGCTA CACTTGCCAG CGCCCTAGCG CCCGCTCCTT TCGCTTTCTT CCCTTCCTTT |
| | 6841 CTCGCCACGT TCGCCGGCTT TCCCCGTCAA GCTCTAAATC GGGGGCTCCC TTTAGGGTTC |
| | 6901 CGATTTAGTG CTTTACGGCA CCTCGACCCC AAAAAACTTG ATTAGGGTGA TGGTTCACGT |
| | 6961 AGTGGGCCAT CGCCCTGATA GACGGTTTTT CGCCCTTTGA CGTTGGAGTC CACGTTCTTT |
| | 7021 AATAGTGGAC TCTTGTTCCA AACTGGAACA ACACTCAACC CTATCTCGGT CTATTCTTTT |
| | 7081 GATTTATAAG GGATTTTGCC GATTTCGGCC TATTGGTTAA AAAATGAGCT GATTTAACAA |
| | 7141 AAATTTAACG CGAATTAATT CTGTGGAATG TGTGTCAGTT AGGGTGTGGA AAGTCCCCAG |
| | 7201 GCTCCCCAGC AGGCAGAAGT ATGCAAAGCA TGCATCTCAA TTAGTCAGCA ACCAGGTGTG |
| | 7261 GAAAGTCCCC AGGCTCCCCA GCAGGCAGAA GTATGCAAAG CATGCATCTC AATTAGTCAG |
| | 7321 CAACCATAGT CCCGCCCCTA ACTCCGCCCA TCCCGCCCCT AACTCCGCCC AGTTCCGCCC |
| | 7381 ATTCTCCGCC CCATGGCTGA CTAATTTTTT TTATTTATGC AGAGGCCGAG GCCGCCTCTG |
| | 7441 CCTCTGAGCT ATTCCAGAAG TAGTGAGGAG GCTTTTTTGG AGGCCTAGGC TTTTGCAAAA |
| | 7501 AGCTCCCGGG AGCTTGTATA TCCATTTTCG GATCTGATCA GCACGTGTTG ACAATTAATC |
| | 7561 ATCGGCATAG TATATCGGCA TAGTATAATA CGACAAGGTG AGGAACTAAA CCATGGCCAA |
| | 7621 GTTGACCAGT GCCGTTCCGG TGCTCACCGC GCGCGACGTC GCCGGAGCGG TCGAGTTCTG |
| | 7681 GACCGACCGG CTCGGGTTCT CCCGGGACTT CGTGGAGGAC GACTTCGCCG GTGTGGTCCG |
| | 7741 GGACGACGTG ACCCTGTTCA TCAGCGCGGT CCAGGACCAG GTGGTGCCGG ACAACACCCT |
| | 7801 GGCCTGGGTG TGGGTGCGCG GCCTGGACGA GCTGTACGCC GAGTGGTCGG AGGTCGTGTC |
| | 7861 CACGAACTTC CGGGACGCCT CCGGGCCGGC CATGACCGAG ATCGGCGAGC AGCCGTGGGG |
| | 7921 GCGGGAGTTC GCCCTGCGCG ACCCGGCCGG CAACTGCGTG CACTTCGTGG CCGAGGAGCA |
| | 7981 GGACTGACAC GTGCTACGAG ATTTCGATTC CACCGCCGCC TTCTATGAAA GGTTGGGCTT |
| | 8041 CGGAATCGTT TTCCGGGACG CCGGCTGGAT GATCCTCCAG CGCGGGGATC TCATGCTGGA |
| | 8101 GTTCTTCGCC CACCCCAACT TGTTTATTGC AGCTTATAAT GGTTACAAAT AAAGCAATAG |
| | 8161 CATCACAAAT TTCACAAATA AAGCATTTTT TTCACTGCAT TCTAGTTGTG GTTTGTCCAA |
| | 8221 ACTCATCAAT GTATCTTATC ATGTCTGTAT ACCGTCGACC TCTAGCTAGA GCTTGGCGTA |
| | 8281 ATCATGGTCA TAGCTGTTTC CTGTGTGAAA TTGTTATCCG CTCACAATTC CACACAACAT |
| | 8341 ACGAGCCGGA AGCATAAAGT GTAAAGCCTG GGGTGCCTAA TGAGTGAGCT AACTCACATT |
| | 8401 AATTGCGTTG CGCTCACTGC CCGCTTTCCA GTCGGGAAAC CTGTCGTGCC AGCTGCATTA |
| | 8461 ATGAATCGGC CAACGCGCGG GGAGAGGCGG TTTGCGTATT GGGCGCTCTT CCGCTTCCTC |
| | 8521 GCTCACTGAC TCGCTGCGCT CGGTCGTTCG GCTGCGGCGA GCGGTATCAG CTCACTCAAA |
| | 8581 GGCGGTAATA CGGTTATCCA CAGAATCAGG GGATAACGCA GGAAAGAACA TGTGAGCAAA |
| | 8641 AGGCCAGCAA AAGGCCAGGA ACCGTAAAAA GGCCGCGTTG CTGGCGTTTT TCCATAGGCT |
| | 8701 CCGCCCCCCT GACGAGCATC ACAAAAATCG ACGCTCAAGT CAGAGGTGGC GAAACCCGAC |
| | 8761 AGGACTATAA AGATACCAGG CGTTTCCCCC TGGAAGCTCC CTCGTGCGCT CTCCTGTTCC |
| | 8821 GACCCTGCCG CTTACCGGAT ACCTGTCCGC CTTTCTCCCT TCGGGAAGCG TGGCGCTTTC |
| | 8881 TCATAGCTCA CGCTGTAGGT ATCTCAGTTC GGTGTAGGTC GTTCGCTCCA AGCTGGGCTG |
| | 8941 TGTGCACGAA CCCCCCGTTC AGCCCGACCG CTGCGCCTTA TCCGGTAACT ATCGTCTTGA |
| | 9001 GTCCAACCCG GTAAGACACG ACTTATCGCC ACTGGCAGCA GCCACTGGTA ACAGGATTAG |
| | 9061 CAGAGCGAGG TATGTAGGCG GTGCTACAGA GTTCTTGAAG TGGTGGCCTA ACTACGGCTA |
| | 9121 CACTAGAAGA ACAGTATTTG GTATCTGCGC TCTGCTGAAG CCAGTTACCT TCGGAAAAAG |
| | 9181 AGTTGGTAGC TCTTGATCCG GCAAACAAAC CACCGCTGGT AGCGGTGGTT TTTTTGTTTG |
| | 9241 CAAGCAGCAG ATTACGCGCA GAAAAAAAGG ATCTCAAGAA GATCCTTTGA TCTTTTCTAC |
| | 9301 GGGGTCTGAC GCTCAGTGGA ACGAAAACTC ACGTTAAGGG ATTTTGGTCA TGAGATTATC |
| | 9361 AAAAAGGATC TTCACCTAGA TCCTTTTAAA TTAAAAATGA AGTTTTAAAT CAATCTAAAG |
| | 9421 TATATATGAG TAAACTTGGT CTGACAGTTA CCAATGCTTA ATCAGTGAGG CACCTATCTC |
| | 9481 AGCGATCTGT CTATTTCGTT CATCCATAGT TGCCTGACTC CCCGTCGTGT AGATAACTAC |
| | 9541 GATACGGGAG GGCTTACCAT CTGGCCCCAG TGCTGCAATG ATACCGCGAG ACCCACGCTC |
| | 9601 ACCGGCTCCA GATTTATCAG CAATAAACCA GCCAGCCGGA AGGGCCGAGC GCAGAAGTGG |
| | 9661 TCCTGCAACT TTATCCGCCT CCATCCAGTC TATTAATTGT TGCCGGGAAG CTAGAGTAAG |
| | 9721 TAGTTCGCCA GTTAATAGTT TGCGCAACGT TGTTGCCATT GCTACAGGCA TCGTGGTGTC |
| | 9781 ACGCTCGTCG TTTGGTATGG CTTCATTCAG CTCCGGTTCC CAACGATCAA GGCGAGTTAC |
| | 9841 ATGATCCCCC ATGTTGTGCA AAAAAGCGGT TAGCTCCTTC GGTCCTCCGA TCGTTGTCAG |
| | 9901 AAGTAAGTTG GCCGCAGTGT TATCACTCAT GGTTATGGCA GCACTGCATA ATTCTCTTAC |
| | 9961 TGTCATGCCA TCCGTAAGAT GCTTTTCTGT GACTGGTGAG TACTCAACCA AGTCATTCTG |
| | 10021 AGAATAGTGT ATGCGGCGAC CGAGTTGCTC TTGCCCGGCG TCAATACGGG ATAATACCGC |
| | 10081 GCCACATAGC AGAACTTTAA AAGTGCTCAT CATTGGAAAA CGTTCTTCGG GGCGAAAACT |
| | 10141 CTCAAGGATC TTACCGCTGT TGAGATCCAG TTCGATGTAA CCCACTCGTG CACCCAACTG |
| | 10201 ATCTTCAGCA TCTTTTACTT TCACCAGCGT TTCTGGGTGA GCAAAAACAG GAAGGCAAAA |
| | 10261 TGCCGCAAAA AAGGGAATAA GGGCGACACG GAAATGTTGA ATACTCATAC TCTTCCTTTT |
| | 10321 TCAATATTAT TGAAGCATTT ATCAGGGTTA TTGTCTCATG AGCGGATACA TATTTGAATG |
| | 10381 TATTTAGAAA AATAAACAAA TAGGGGTTCC GCGCACATTT CCCCGAAAAG TGCCACCTGA |
| | 10441 C |

//

pLL3.0

| LOCUS PLENTILOX | | 6027 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | gene | 5034..5894 |
| | | /note=“AmpR” |
| | rep_origin | 4216..4889 |
| | | /note=“pUC” |
| | misc_recomb | 2710..2743 |
| | | /note=“LoxP” |
| | misc_recomb | 2827..2860 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 2915..3504 |
| | | /note=“WRE element” |
| | LTR | 3524..4213 |
| | | /note=“3′ SIN LTR” |

| | |
|---|---|
| BASE COUNT 1612 A | 1408 C 1518 G 1489 T 0 OTHER |
| ORIGIN - | |

| (SEQ ID NO:2) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTAACG CGCTAGCCGT |
| | 2641 TAATTAAGCC TCGAGGTCGA CGGTATCGAT AAGCTCGCTT CACGAGATTC CAGCAGGTCG |
| | 2701 AGGGACCTAA TAACTTCGTA TAGCATACAT TATACGAAGT TATATTAAGG GTTCCAAGCT |
| | 2761 TAAGCGGCCG CCGATGCATG CCCCGGGATG GCGCGCCATG GATCCGCGAA TTCGTCGAGG |
| | 2821 GACCTAATAA CTTCGTATAG CATACATTAT ACGAAGTTAT ACATGTTTAA GGGTTCCGGT |
| | 2881 TCCACTAGGT ACAATTCGAT ATCAAGCTTA TCGATAATCA ACCTCTGGAT TACAAAATTT |
| | 2941 GTGAAAGATT GACTGGTATT CTTAACTATG TTGCTCCTTT TACGCTATGT GGATACGCTG |
| | 3001 CTTTAATGCC TTTGTATCAT GCTATTGCTT CCCGTATGGC TTTCATTTTC TCCTCCTTGT |
| | 3061 ATAAATCCTG GTTGCTGTCT CTTTATGAGG AGTTGTGGCC CGTTGTCAGG CAACGTGGCG |
| | 3121 TGGTGTGCAC TGTGTTTGCT GACGCAACCC CCACTGGTTG GGGCATTGCC ACCACCTGTC |
| | 3181 AGCTCCTTTC CGGGACTTTC GCTTTCCCCC TCCCTATTGC CACGGCGGAA CTCATCGCCG |
| | 3241 CCTGCCTTGC CCGCTGCTGG ACAGGGGCTC GGCTGTTGGG CACTGACAAT TCCGTGGTGT |
| | 3301 TGTCGGGGAA ATCATCGTCC TTTCCTTGGC TGCTCGCCTG TGTTGCCACC TGGATTCTGC |
| | 3361 GCGGGACGTC CTTCTGCTAC GTCCCTTCGG CCCTCAATCC AGCGGACCTT CCTTCCCGCG |
| | 3421 GCCTGCTGCC GGCTCTGCGG CCTCTTCCGC GTCTTCGCCT TCGCCCTCAG ACGAGTCGGA |
| | 3481 TCTCCCTTTG GGCCGCCTCC CCGCATCGAT ACCGTCGACC TCGATCGAGA CCTAGAAAAA |
| | 3541 CATGGAGCAA TCACAAGTAG CAATACAGCA GCTACCAATG CTGATTGTGC CTGGCTAGAA |
| | 3601 GCACAAGAGG AGGAGGAGGT GGGTTTTCCA GTCACACCTC AGGTACCTTT AAGACCAATG |
| | 3661 ACTTACAAGG CAGCTGTAGA TCTTAGCCAC TTTTTAAAAG AAAAGGGGGG ACTGGAAGGG |
| | 3721 CTAATTCACT CCCAACGAAG ACAAGATATC CTTGATCTGT GGATCTACCA CACACAAGGC |
| | 3781 TACTTCCCTG ATTGGCAGAA CTACACACCA GGGCCAGGGA TCAGATATCC ACTGACCTTT |
| | 3841 GGATGGTGCT ACAAGCTAGT ACCAGTTGAG CAAGAGAAGG TAGAAGAAGC CAATGAAGGA |
| | 3901 GAGAACACCC GCTTGTTACA CCCTGTGAGC CTGCATGGGA TGGATGACCC GGAGAGAGAA |
| | 3961 GTATTAGAGT GGAGGTTTGA CAGCCGCCTA GCATTTCATC ACATGGCCCG AGAGCTGCAT |
| | 4021 CCGGACTGTA CTGGGTCTCT CTGGTTAGAC CAGATCTGAG CCTGGGAGCT CTCTGGCTAA |
| | 4081 CTAGGGAACC CACTGCTTAA GCCTCAATAA AGCTTGCCTT GAGTGCTTCA AGTAGTGTGT |
| | 4141 GCCCGTCTGT TGTGTGACTC TGGTAACTAG AGATCCCTCA GACCCTTTTA GTCAGTGTGG |
| | 4201 AAAATCTCTA GCAGCATGTG AGCAAAAGGC CAGCAAAAGG CCAGGAACCG TAAAAAGGCC |
| | 4261 GCGTTGCTGG CGTTTTTCCA TAGGCTCCGC CCCCCTGACG AGCATCACAA AAATCGACGC |
| | 4321 TCAAGTCAGA GGTGGCGAAA CCCGACAGGA CTATAAAGAT ACCAGGCGTT TCCCCCTGGA |
| | 4381 AGCTCCCTCG TGCGCTCTCC TGTTCCGACC CTGCCGCTTA CCGGATACCT GTCCGCCTTT |
| | 4441 CTCCCTTCGG GAAGCGTGGC GCTTTCTCAT AGCTCACGCT GTAGGTATCT CAGTTCGGTG |
| | 4501 TAGGTCGTTC GCTCCAAGCT GGGCTGTGTG CACGAACCCC CCGTTCAGCC CGACCGCTGC |
| | 4561 GCCTTATCCG GTAACTATCG TCTTGAGTCC AACCCGGTAA GACACGACTT ATCGCCACTG |
| | 4621 GCAGCAGCCA CTGGTAACAG GATTAGCAGA GCGAGGTATG TAGGCGGTGC TACAGAGTTC |
| | 4681 TTGAAGTGGT GGCCTAACTA CGGCTACACT AGAAGAACAG TATTTGGTAT CTGCGCTCTG |
| | 4741 CTGAAGCCAG TTACCTTCGG AAAAAGAGTT GGTAGCTCTT GATCCGGCAA ACAAACCACC |
| | 4801 GCTGGTAGCG GTGGTTTTTT TGTTTGCAAG CAGCAGATTA CGCGCAGAAA AAAAGGATCT |
| | 4861 CAAGAAGATC CTTTGATCTT TTCTACGGGG TCTGACGCTC AGTGGAACGA AAACTCACGT |
| | 4921 TAAGGGATTT TGGTCATGAG ATTATCAAAA AGGATCTTCA CCTAGATCCT TTTAAATTAA |
| | 4981 AAATGAAGTT TTAAATCAAT CTAAAGTATA TATGAGTAAA CTTGGTCTGA CAGTTACCAA |
| | 5041 TGCTTAATCA GTGAGGCACC TATCTCAGCG ATCTGTCTAT TTCGTTCATC CATAGTTGCC |
| | 5101 TGACTCCCCG TCGTGTAGAT AACTACGATA CGGGAGGGCT TACCATCTGG CCCCAGTGCT |
| | 5161 GCAATGATAC CGCGAGACCC ACGCTCACCG GCTCCAGATT TATCAGCAAT AAACCAGCCA |
| | 5221 GCCGGAAGGG CCGAGCGCAG AAGTGGTCCT GCAACTTTAT CCGCCTCCAT CCAGTCTATT |
| | 5281 AATTGTTGCC GGGAAGCTAG AGTAAGTAGT TCGCCAGTTA ATAGTTTGCG CAACGTTGTT |
| | 5341 GCCATTGCTA CAGGCATCGT GGTGTCACGC TCGTCGTTTG GTATGGCTTC ATTCAGCTCC |
| | 5401 GGTTCCCAAC GATCAAGGCG ACTTACATGA TCCCCCATGT TGTGCAAAAA AGCGGTTAGC |
| | 5461 TCCTTCGGTC CTCCGATCGT TGTCAGAAGT AAGTTGGCCG CAGTGTTATC ACTCATGGTT |
| | 5521 ATGGCAGCAC TGCATAATTC TCTTACTGTC ATGCCATCCG TAAGATGCTT TTCTGTGACT |
| | 5581 GGTGAGTACT CAACCAAGTC ATTCTGAGAA TAGTGTATGC GGCGACCGAG TTGCTCTTGC |
| | 5641 CCGGCGTCAA TACGGGATAA TACCGCGCCA CATAGCAGAA CTTTAAAAGT GCTCATCATT |
| | 5701 GGAAAACGTT CTTCGGGGCG AAAACTCTCA AGGATCTTAC CGCTGTTGAG ATCCAGTTCG |
| | 5761 ATGTAACCCA CTCGTGCACC CAACTGATCT TCAGCATCTT TTACTTTCAC CAGCGTTTCT |
| | 5821 GGGTGAGCAA AAACAGGAAG GCAAAATGCC GCAAAAAAGG GAATAAGGGC GACACGGAAA |
| | 5881 TGTTGAATAC TCATACTCTT CCTTTTTCAA TATTATTGAA GCATTTATCA GGGTTATTGT |
| | 5941 CTCATGAGCG GATACATATT TGAATGTATT TAGAAAAATA AACAAATAGG GGTTCCGCGC |
| | 6001 ACATTTCCCC GAAAAGTGCC ACCTGAC |

//

pLL3.1

| LOCUS PLENTILOX | | 6748 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | misc_recomb | 3548..3581 |
| | | /note=“LoxP” |
| | gene | 5755..6615 |
| | | /note=“AmpR” |
| | rep_origin | 4937..5610 |
| | | /note=“pUC” |
| | misc_recomb | 2710..2745 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 3636..4225 |
| | | /note=“WRE element” |
| | LTR | 4245..4934 |
| | | /note=“3′ SIN LTR” |
| | gene | 2772..3494 |
| | | /note=“EGFP” |

| | |
|---|---|
| BASE COUNT 1785 A | 1651 C 1721 G 1591 T 0 OTHER |
| ORIGIN - | |

| (SEQ ID NO:3) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTAACG CGCTAGCCGT |
| | 2641 TAATTAAGCC TCGAGGTCGA CGGTATCGAT AAGCTCGCTT CACGAGATTC CAGCAGGTCG |
| | 2701 AGGGACCTAA TAACTTCGTA TAGCATACAT TATACGAAGT TATATTAAGG GTTCCAAGCT |
| | 2761 TAAGCGGCCG CGCCACCATG GTGAGCAAGG GCGAGGAGCT GTTCACCGGG GTGGTGCCCA |
| | 2821 TCCTGGTCGA GCTGGACGGC GACGTAAACG GCCACAAGTT CAGCGTGTCC GGCGAGGGCG |
| | 2881 AGGGCGATGC CACCTACGGC AAGCTGACCC TGAAGTTCAT CTGCACCACC GGCAAGCTGC |
| | 2941 CCGTGCCCTG GCCCACCCTC GTGACCACCC TGACCTACGG CGTGCAGTGC TTCAGCCGCT |
| | 3001 ACCCCGACCA CATGAAGCAG CACGACTTCT TCAAGTCCGC CATGCCCGAA GGCTACGTCC |
| | 3061 AGGAGCGCAC CATCTTCTTC AAGGACGACG GCAACTACAA GACCCGCGCC GAGGTGAAGT |
| | 3121 TCGAGGGCGA CACCCTGGTG AACCGCATCG AGCTGAAGGG CATCGACTTC AAGGAGGACG |
| | 3181 GCAACATCCT GGGGCACAAG CTGGAGTACA ACTACAACAG CCACAACGTC TATATCATGG |
| | 3241 CCGACAAGCA GAAGAACGGC ATCAAGGTGA ACTTCAAGAT CCGCCACAAC ATCGAGGACG |
| | 3301 GCAGCGTGCA GCTCGCCGAC CACTACCAGC AGAACACCCC CATCGGCGAC GGCCCCGTGC |
| | 3361 TGCTGCCCGA CAACCACTAC CTGAGCACCC AGTCCGCCCT GAGCAAAGAC CCCAACGAGA |
| | 3421 AGCGCGATCA CATGGTCCTG CTGGAGTTCG TGACCGCCGC CGGGATCACT CTCGGCATGG |
| | 3481 ACGAGCTGTA CAAGATGCAT GCCCCGGGAT GGCGCGCCAT GGATCCGCGA ATTCGTCGAG |
| | 3541 GGACCTAATA ACTTCGTATA GCATACATTA TACGAAGTTA TACATGTTTA AGGGTTCCGG |
| | 3601 TTCCACTAGG TACAATTCGA TATCAAGCTT ATCGATAATC AACCTCTGGA TTACAAAATT |
| | 3661 TGTGAAAGAT TGACTGGTAT TCTTAACTAT GTTGCTCCTT TTACGCTATG TGGATACGCT |
| | 3721 GCTTTAATGC CTTTGTATCA TGCTATTGCT TCCCGTATGG CTTTCATTTT CTCCTCCTTG |
| | 3781 TATAAATCCT GGTTGCTGTC TCTTTATGAG GAGTTGTGGC CCGTTGTCAG GCAACGTGGC |
| | 3841 GTGGTGTGCA CTGTGTTTGC TGACGCAACC CCCACTGGTT GGGGCATTGC CACCACCTGT |
| | 3901 CAGCTCCTTT CCGGGACTTT CGCTTTCCCC CTCCCTATTG CCACGGCGGA ACTCATCGCC |
| | 3961 GCCTGCCTTG CCCGCTGCTG GACAGGGGCT CGGCTGTTGG GCACTGACAA TTCCGTGGTG |
| | 4021 TTGTCGGGGA AATCATCGTC CTTTCCTTGG CTGCTCGCCT GTGTTGCCAC CTGGATTCTG |
| | 4081 CGCGGGACGT CCTTCTGCTA CGTCCCTTCG GCCCTCAATC CAGCGGACCT TCCTTCCCGC |
| | 4141 GGCCTGCTGC CGGCTCTGCG GCCTCTTCCG CGTCTTCGCC TTCGCCCTCA GACGAGTCGG |
| | 4201 ATCTCCCTTT GGGCCGCCTC CCCGCATCGA TACCGTCGAC CTCGATCGAG ACCTAGAAAA |
| | 4261 ACATGGAGCA ATCACAAGTA GCAATACAGC AGCTACCAAT GCTGATTGTG CCTGGCTAGA |
| | 4321 AGCACAAGAG GAGGAGGAGG TGGGTTTTCC AGTCACACCT CAGGTACCTT TAAGACCAAT |
| | 4381 GACTTACAAG GCAGCTGTAG ATCTTAGCCA CTTTTTAAAA GAAAAGGGGG GACTGGAAGG |
| | 4441 GCTAATTCAC TCCCAACGAA GACAAGATAT CCTTGATCTG TGGATCTACC ACACACAAGG |
| | 4501 CTACTTCCCT GATTGGCAGA ACTACACACC AGGGCCAGGG ATCAGATATC CACTGACCTT |
| | 4561 TGGATGGTGC TACAAGCTAG TACCAGTTGA GCAAGAGAAG GTAGAAGAAG CCAATGAAGG |
| | 4621 AGAGAACACC CGCTTGTTAC ACCCTGTGAG CCTGCATGGG ATGGATGACC CGGAGAGAGA |
| | 4681 AGTATTAGAG TGGAGGTTTG ACAGCCGCCT AGCATTTCAT CACATGGCCC GAGAGCTGCA |
| | 4741 TCCGGACTGT ACTGGGTCTC TCTGGTTAGA CCAGATCTGA GCCTGGGAGC TCTCTGGCTA |
| | 4801 ACTAGGGAAC CCACTGCTTA AGCCTCAATA AAGCTTGCCT TGAGTGCTTC AAGTAGTGTG |
| | 4861 TGCCCGTCTG TTGTGTGACT CTGGTAACTA GAGATCCCTC AGACCCTTTT AGTCAGTGTG |
| | 4921 GAAAATCTCT AGCAGCATGT GAGCAAAAGG CCAGCAAAAG GCCAGGAACC GTAAAAAGGC |
| | 4981 CGCGTTGCTG GCGTTTTTCC ATAGGCTCCG CCCCCCTGAC GAGCATCACA AAAATCGACG |
| | 5041 CTCAAGTCAG AGGTGGCGAA ACCCGACAGG ACTATAAAGA TACCAGGCGT TTCCCCCTGG |
| | 5101 AAGCTCCCTC GTGCGCTCTC CTGTTCCGAC CCTGCCGCTT ACCGGATACC TGTCCGCCTT |
| | 5161 TCTCCCTTCG GGAAGCGTGG CGCTTTCTCA TAGCTCACGC TGTAGGTATC TCAGTTCGGT |
| | 5221 GTAGGTCGTT CGCTCCAAGC TGGGCTGTGT GCACGAACCC CCCGTTCAGC CCGACCGCTG |
| | 5281 CGCCTTATCC GGTAACTATC GTCTTGAGTC CAACCCGGTA AGACAGGACT TATCGCCACT |
| | 5341 GGCAGCAGCC ACTGGTAACA GGATTAGCAG AGCGAGGTAT GTAGGCGGTG CTACAGAGTT |
| | 5401 CTTGAAGTGG TGGCCTAACT ACGGCTACAC TAGAAGAACA GTATTTGGTA TCTGCGCTCT |
| | 5461 GCTGAAGCCA GTTACCTTCG GAAAAAGAGT TGGTAGCTCT TGATCCGGCA AACAAACCAC |
| | 5521 CGCTGGTAGC GGTGGTTTTT TTGTTTGCAA GCAGCAGATT ACGCGCAGAA AAAAAGGATC |
| | 5581 TCAAGAAGAT CCTTTGATCT TTTCTACGGG GTCTGACGCT CAGTGGAACG AAAACTCACG |
| | 5641 TTAAGGGATT TTGGTCATGA GATTATCAAA AAGGATCTTC ACCTAGATCC TTTTAAATTA |
| | 5701 AAAATGAAGT TTTAAATCAA TCTAAAGTAT ATATGAGTAA ACTTGGTCTG ACAGTTACCA |
| | 5761 ATGCTTAATC AGTGAGGCAC CTATCTCAGC GATCTGTCTA TTTCGTTCAT CCATAGTTGC |
| | 5821 CTGACTCCCC GTCGTGTAGA TAACTACGAT ACGGGAGGGC TTACCATCTG GCCCCAGTGC |
| | 5881 TGCAATGATA CCGCGAGACC CACGCTCACC GGCTCCAGAT TTATCAGCAA TAAACCAGCC |
| | 5941 AGCCGGAAGG GCCGAGCGCA GAAGTGGTCC TGCAACTTTA TCCGCCTCCA TCCAGTCTAT |
| | 6001 TAATTGTTGC CGGGAAGCTA GAGTAAGTAG TTCGCCAGTT AATAGTTTGC GCAACGTTGT |
| | 6061 TGCCATTGCT ACAGGCATCG TGGTGTCACG CTCGTCGTTT GGTATGGCTT CATTCAGCTC |
| | 6121 CGGTTCCCAA CGATCAAGGC GAGTTACATG ATCCCCCATG TTGTGCAAAA AAGCGGTTAG |
| | 6181 CTCCTTCGGT CCTCCGATCG TTGTCAGAAG TAAGTTGGCC GCAGTGTTAT CACTCATGGT |
| | 6241 TATGGCAGCA CTGCATAATT CTCTTACTGT CATGCCATCC GTAAGATGCT TTTCTGTGAC |
| | 6301 TGGTGAGTAC TCAACCAAGT CATTCTGAGA ATAGTGTATG CGGCGACCGA GTTGCTCTTG |
| | 6361 CCCGGCGTCA ATACGGGATA ATACCGCGCC ACATAGCAGA ACTTTAAAAG TCCTCATCAT |
| | 6421 TGGAAAACGT TCTTCGGGGC GAAAACTCTC AAGGATCTTA CCGCTGTTGA GATCCAGTTC |
| | 6481 GATGTAACCC ACTCGTGCAC CCAACTGATC TTCAGCATCT TTTACTTTCA CCAGCGTTTC |
| | 6541 TGGGTGAGCA AAAACAGGAA GGCAAAATGC CGCAAAAAAG GGAATAAGGG CGACACGGAA |
| | 6601 ATGTTGAATA CTCATACTCT TCCTTTTTCA ATATTATTGA AGCATTTATC AGGGTTATTG |
| | 6661 TCTCATGAGC GGATACATAT TTGAATGTAT TTAGAAAAAT AAACAAATAG GGGTTCCGCG |
| | 6721 CACATTTCCC CGAAAAGTGC CACCTGAC |

//

pLL3.2

| LOCUS PLENTILOX | | 6706 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | misc_recomb | 3506..3539 |
| | | /note=“LoxP” |
| | gene | 5713..6573 |
| | | /note=“AmpR” |
| | rep_origin | 4895..5568 |
| | | /note=“pUC” |
| | misc_recomb | 2710..2745 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 3594..4183 |
| | | /note=“WRE element” |
| | LTR | 4203..4892 |
| | | /note=“3′SIN LTR” |
| | gene | 2772..3452 |
| | | /note=“dsRed2” |

| | |
|---|---|
| BASE COUNT 1755 A | 1638 C 1722 G 1591 T 0 OTHER |
| ORIGIN - | |

| (SEQ ID NO:4) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTAACG CGCTAGCCGT |
| | 2641 TAATTAAGCC TCGAGGTCGA CGGTATCGAT AAGCTCGCTT CACGAGATTC CAGCAGGTCG |
| | 2701 AGGGACCTAA TAACTTCGTA TAGCATACAT TATACGAAGT TATATTAAGG GTTCCAAGCT |
| | 2761 TAAGCGGCCG CGCCACCATG GCCTCCTCCG AGAACGTCAT CACCGAGTTC ATGCGCTTCA |
| | 2821 AGGTGCGCAT GGAGGGCACC GTGAACGGCC ACGAGTTCGA GATCGAGGGC GAGGGCGAGG |
| | 2881 GCCGCCCCTA CGAGGGCCAC AACACCGTGA AGCTGAAGGT GACCAAGGGC GGCCCCCTGC |
| | 2941 CCTTCGCCTG GGACATCCTG TCCCCCCAGT TCCAGTACGG CTCCAAGGTG TACGTGAAGC |
| | 3001 ACCCCGCCGA CATCCCCGAC TACAAGAAGC TGTCCTTCCC CGAGGGCTTC AAGTGGGAGC |
| | 3061 GCGTGATGAA CTTCGAGGAC GGCGGCGTGG CGACCGTGAC CCAGGACTCC TCCCTGCAGG |
| | 3121 ACGGCTGCTT CATCTACAAG GTGAAGTTCA TCGGCGTGAA CTTCCCCTCC GACGGCCCCG |
| | 3181 TGATGCAGAA GAAGACCATG GGCTGGGAGG CCTCCACCGA GCGCCTGTAC CCCCGCGACG |
| | 3241 GCGTGCTGAA GGGCGAGACC CACAAGGCCC TGAAGCTGAA GGACGGCGGC CACTACCTGG |
| | 3301 TGGAGTTCAA GTCCATCTAC ATGGCCAAGA AGCCCGTGCA GCTGCCCGGC TACTACTACG |
| | 3361 TGGACGCCAA GCTGGACATC ACCTCCCACA ACGAGGACTA CACCATCGTG GAGCAGTACG |
| | 3421 AGCGCACCGA GGGCCGCCAC CACCTGTTCC TGATGCATGC CCCGGGATGG CGCGCCATGG |
| | 3481 ATCCGCGAAT TCGTCGAGGG ACCTAATAAC TTCGTATAGC ATACATTATA CGAAGTTATA |
| | 3541 CATGTTTAAG GGTTCCGGTT CCACTAGGTA CAATTCGATA TCAAGCTTAT CGATAATCAA |
| | 3601 CCTCTGGATT ACAAAATTTG TGAAAGATTG ACTGGTATTC TTAACTATGT TGCTCCTTTT |
| | 3661 ACGCTATGTG GATACGCTGC TTTAATGCCT TTGTATCATG CTATTGCTTC CCGTATGGCT |
| | 3721 TTCATTTTCT CCTCCTTGTA TAAATCCTGG TTGCTGTCTC TTTATGAGGA GTTGTGGCCC |
| | 3781 GTTGTCAGGC AACGTGGCGT GGTGTGCACT GTGTTTGCTG ACGCAACCCC CACTGGTTGG |
| | 3841 GGCATTGCCA CCACCTGTCA GCTCCTTTCC GGGACTTTCG CTTTCCCCCT CCCTATTGCC |
| | 3901 ACGGCGGAAC TCATCGCCGC CTGCCTTGCC CGCTGCTGGA CAGGGGCTCG GCTGTTGGGC |
| | 3961 ACTGACAATT CCGTGGTGTT GTCGGGGAAA TCATCGTCCT TTCCTTGGCT GCTCGCCTGT |
| | 4021 GTTGCCACCT GGATTCTGCG CGGGACGTCC TTCTGCTACG TCCCTTCGGC CCTCAATCCA |
| | 4081 GCGGACCTTC CTTCCCGCGG CCTGCTGCCG GCTCTGCGGC CTCTTCCGCG TCTTCGCCTT |
| | 4141 CGCCCTCAGA CGAGTCGGAT CTCCCTTTGG GCCGCCTCCC CGCATCGATA CCGTCGACCT |
| | 4201 CGATCGAGAC CTAGAAAAAC ATGGAGCAAT CACAAGTAGC AATACAGCAG CTACCAATGC |
| | 4261 TGATTGTGCC TGGCTAGAAG CACAAGAGGA GGAGGAGGTG GGTTTTCCAG TCACACCTCA |
| | 4321 GGTACCTTTA AGACCAATGA CTTACAAGGC AGCTGTAGAT CTTAGCCACT TTTTAAAAGA |
| | 4381 AAAGGGGGGA CTGGAAGGGC TAATTCACTC CCAACGAAGA CAAGATATCC TTGATCTGTG |
| | 4441 GATCTACCAC ACACAAGGCT ACTTCCCTGA TTGGCAGAAC TACACACCAG GGCCAGGGAT |
| | 4501 CAGATATCCA CTGACCTTTG GATGGTGCTA CAAGCTAGTA CCAGTTGAGC AAGAGAAGGT |
| | 4561 AGAAGAAGCC AATGAAGGAG AGAACACCCG CTTGTTACAC CCTGTGAGCC TGCATGGGAT |
| | 4621 GGATGACCCG GAGAGAGAAG TATTAGAGTG GAGGTTTGAC AGCCGCCTAG CATTTCATCA |
| | 4681 CATGGCCCGA GAGCTGCATC CGGACTGTAC TGGGTCTCTC TGGTTAGACC AGATCTGAGC |
| | 4741 CTGGGAGCTC TCTGGCTAAC TAGGGAACCC ACTGCTTAAG CCTCAATAAA GCTTGCCTTG |
| | 4801 AGTGCTTCAA GTAGTGTGTG CCCGTCTGTT GTGTGACTCT GGTAACTAGA GATCCCTCAG |
| | 4861 ACCCTTTTAG TCAGTGTGGA AAATCTCTAG CAGCATGTGA GCAAAAGGCC AGCAAAAGGC |
| | 4921 CAGGAACCGT AAAAAGGCCG CGTTGCTGGC GTTTTTCCAT AGGCTCCGCC CCCCTGACGA |
| | 4981 GCATCACAAA AATCGACGCT CAAGTCAGAG GTGGCGAAAC CCGACAGGAC TATAAAGATA |
| | 5041 CCAGGCGTTT CCCCCTGGAA GCTCCCTCGT GCGCTCTCCT GTTCCGACCC TGCCGCTTAC |
| | 5101 CGGATACCTG TCCGCCTTTC TCCCTTCGGG AAGCGTGGCG CTTTCTCATA GCTCACGCTG |
| | 5161 TAGGTATCTC AGTTCGGTGT AGGTCGTTCG CTCCAAGCTG GGCTGTGTGC ACGAACCCCC |
| | 5221 CGTTCAGCCC GACCGCTGCG CCTTATCCGG TAACTATCGT CTTGAGTCCA ACCCGGTAAG |
| | 5281 ACACGACTTA TCGCCACTGG CAGCAGCCAC TGGTAACAGG ATTAGCAGAG CGAGGTATGT |
| | 5341 AGGCGGTGCT ACAGAGTTCT TGAAGTGGTG GCCTAACTAC GGCTACACTA GAAGAACAGT |
| | 5401 ATTTGGTATC TGCGCTCTGC TGAAGCCAGT TACCTTCGGA AAAAGAGTTG GTAGCTCTTG |
| | 5461 ATCCGGCAAA CAAACCACCG CTGGTAGCGG TGGTTTTTTT GTTTGCAAGC AGCAGATTAC |
| | 5521 GCGCAGAAAA AAAGGATCTC AAGAAGATCC TTTGATCTTT TCTACGGGGT CTGACGCTCA |
| | 5581 GTGGAACGAA AACTCACGTT AAGGGATTTT GGTCATGAGA TTATCAAAAA GGATCTTCAC |
| | 5641 CTAGATCCTT TTAAATTAAA AATGAAGTTT TAAATCAATC TAAAGTATAT ATGAGTAAAC |
| | 5701 TTGGTCTGAC AGTTACCAAT GCTTAATCAG TGAGGCACCT ATCTCAGCGA TCTGTCTATT |
| | 5761 TCGTTCATCC ATAGTTGCCT GACTCCCCGT CGTGTAGATA ACTACGATAC GGGAGGGCTT |
| | 5821 ACCATCTGGC CCCAGTGCTG CAATGATACC GCGAGACCCA CGCTCACCGG CTCCAGATTT |
| | 5881 ATCAGCAATA AACCAGCCAG CCGGAAGGGC CGAGCGCAGA AGTGGTCCTG CAACTTTATC |
| | 5941 CGCCTCCATC CAGTCTATTA ATTGTTGCCG GGAAGCTAGA GTAAGTAGTT CGCCAGTTAA |
| | 6001 TAGTTTGCGC AACGTTGTTG CCATTGCTAC AGGCATCGTG GTGTCACGCT CGTCGTTTGG |
| | 6061 TATGGCTTCA TTCAGCTCCG GTTCCCAACG ATCAAGGCGA GTTACATGAT CCCCCATGTT |
| | 6121 GTGCAAAAAA GCGGTTAGCT CCTTCGGTCC TCCGATCGTT GTCAGAAGTA AGTTGGCCGC |
| | 6181 AGTGTTATCA CTCATGGTTA TGGCAGCACT GCATAATTCT CTTACTGTCA TGCCATCCGT |
| | 6241 AAGATGCTTT TCTGTGACTG GTGAGTACTC AACCAAGTCA TTCTGAGAAT AGTGTATGCG |
| | 6301 GCGACCGAGT TGCTCTTGCC CGGCGTCAAT ACGGGATAAT ACCGCGCCAC ATAGCAGAAC |
| | 6361 TTTAAAAGTG CTCATCATTG GAAAACGTTC TTCGGGGCGA AAACTCTCAA GGATCTTACC |
| | 6421 GCTGTTGAGA TCCAGTTCGA TGTAACCCAC TCGTGCACCC AACTGATCTT CAGCATCTTT |
| | 6481 TACTTTCACC AGCGTTTCTG GGTGAGCAAA AACAGGAAGG CAAAATGCCG CAAAAAAGGG |
| | 6541 AATAAGGGCG ACACGGAAAT GTTGAATACT CATACTCTTC CTTTTTCAAT ATTATTGAAG |
| | 6601 CATTTATCAG GGTTATTGTC TCATGAGCGG ATACATATTT GAATGTATTT AGAAAAATAA |
| | 6661 ACAAATAGGG GTTCCGCGCA CATTTCCCCG AAAAGTGCCA CCTGAC |

//

pLL3.3

| LOCUS PLENTILOX | | 7248 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | gene | 6255..7115 |
| | | /note=“AmpR” |
| | rep_origin | 5437..6110 |
| | | /note=“pUC” |
| | misc_recomb | 3931..3966 |
| | | /note=“Lox 1” |
| | misc_recomb | 4048..4081 |
| | | /note=“Lox2” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 4136..4725 |
| | | /note=“WRE element” |
| | LTR | 4745..5434 |
| | | /note=“3′ SIN LTR” |
| | frag | 2627..3847 |
| | | /note=“13 to 1233 of pUB6/V5-HisA” |
| | promoter | 2632..3841 |
| | | /note=“UbC promoter” |

| | |
|---|---|
| BASE COUNT 1815 A | 1695 C 1947 G 1791 T 0 OTHER |
| ORIGIN - | |

| (SEQ ID NO:5) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAPAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTGATC TGGCCTCCGC |
| | 2641 GCCGGGTTTT GGCGCCTCCC GCGGGCGCCC CCCTCCTCAC GGCGAGCGCT GCCACGTCAG |
| | 2701 ACGAAGGGCG CAGGAGCGTC CTGATCCTTC CGCCCGGACG CTCAGGACAG CGGCCCGCTG |
| | 2761 CTCATAAGAC TCGGCCTTAG AACCCCAGTA TCAGCAGAAG GACATTTTAG GACGGGACTT |
| | 2821 GGGTGACTCT AGGGCACTGG TTTTCTTTCC AGAGAGCGGA ACAGGCGAGG AAAAGTAGTC |
| | 2881 CCTTCTCGGC GATTCTGCGG AGGGATCTCC GTGGGGCGGT GAACGCCGAT GATTATATAA |
| | 2941 GGACGCGCCG GGTGTGGCAC AGCTAGTTCC GTCGCAGCCG GGATTTGGGT CGCGGTTCTT |
| | 3001 GTTTGTGGAT CGCTGTGATC GTCACTTGGT GAGTAGCGGG CTGCTGGGCT GGCCGGGGCT |
| | 3061 TTCGTGGCCG CCGGGCCGCT CGGTGGGACG GAAGCGTGTG GAGAGACCGC CAAGGGCTGT |
| | 3121 AGTCTGGGTC CGCGAGCAAG GTTGCCCTGA ACTGGGGGTT GGGGGGAGCG CAGCAAAATG |
| | 3181 GCGGCTGTTC CCGAGTCTTG AATGGAAGAC GCTTGTGAGG CGGGCTGTGA GGTCGTTGAA |
| | 3241 ACAAGGTGGG GGGCATGGTG GGCGGCAAGA ACCCAAGGTC TTGAGGCCTT CGCTAATGCG |
| | 3301 GGAAAGCTCT TATTCGGGTG AGATGGGCTG GGGCACCATC TGGGGACCCT GACGTGAAGT |
| | 3361 TTGTCACTGA CTGGAGAACT CGGTTTGTCG TCTGTTGCGG GGGCGGCAGT TATGCGGTGC |
| | 3421 CGTTGGGCAG TGCACCCGTA CCTTTGGGAG CGCGCGCCCT CGTCGTGTCG TGACGTCACC |
| | 3481 CGTTCTGTTG GCTTATAATG CAGGGTGGGG CCACCTGCCG GTAGGTGTGC GGTAGGCTTT |
| | 3541 TCTCCGTCGC AGGACGCAGG GTTCGGGCCT AGGGTAGGCT CTCCTGAATC GACAGGCGCC |
| | 3601 GGACCTCTGG TGAGGGGAGG GATAAGTGAG GCGTCAGTTT CTTTGGTCGG TTTTATGTAC |
| | 3661 CTATCTTCTT AAGTAGCTGA AGCTCCGGTT TTGAACTATG CGCTCGGGGT TGGCGAGTGT |
| | 3721 GTTTTGTGAA GTTTTTTAGG CACCTTTTGA AATGTAATCA TTTGGGTCAA TATGTAATTT |
| | 3781 TCAGTGTTAG ACTAGTAAAT TGTCCGCTAA ATTCTGGCCG TTTTTGGCTT TTTTGTTAGA |
| | 3841 CGAAGCTAAC GCGCTAGCCG TTAATTAAGC CTCGAGGTCG ACGGTATCGA TAAGCTCGCT |
| | 3901 TCACGAGATT CCAGCAGGTC GAGGGACCTA ATAACTTCGT ATAGCATACA TTATACGAAG |
| | 3961 TTATATTAAG GGTTCCAAGC TTAAGCGGCC GCCGATGCAT GCCCCGGGAT GGCGCGCCAT |
| | 4021 GGATCCGCGA ATTCGTCGAG GGACCTAATA ACTTCGTATA GCATACATTA TACGAAGTTA |
| | 4081 TACATGTTTA AGGGTTCCGG TTCCACTAGG TACAATTCGA TATCAAGCTT ATCGATAATC |
| | 4141 AACCTCTGGA TTACAAAATT TGTGAAAGAT TGACTGGTAT TCTTAACTAT GTTGCTCCTT |
| | 4201 TTACGCTATG TGGATACGCT GCTTTAATGC CTTTGTATCA TGCTATTGCT TCCCGTATGG |
| | 4261 CTTTCATTTT CTCCTCCTTG TATAAATCCT GGTTGCTGTC TCTTTATGAG GAGTTGTGGC |
| | 4321 CCGTTGTCAG GCAACGTGGC GTGGTGTGCA CTGTGTTTGC TGACGCAACC CCCACTGGTT |
| | 4381 GGGGCATTGC CACCACCTGT CAGCTCCTTT CCGGGACTTT CGCTTTCCCC CTCCCTATTG |
| | 4441 CCACGGCGGA ACTCATCGCC GCCTGCCTTG CCCGCTGCTG GACAGGGGCT CGGCTGTTGG |
| | 4501 GCACTGACAA TTCCGTGGTG TTGTCGGGGA AATCATCGTC CTTTCCTTGG CTGCTCGCCT |
| | 4561 GTGTTGCCAC CTGGATTCTG CGCGGGACGT CCTTCTGCTA CGTCCCTTCG GCCCTCAATC |
| | 4621 CAGCGGACCT TCCTTCCCGC GGCCTGCTGC CGGCTCTGCG GCCTCTTCCG CGTCTTCGCC |
| | 4681 TTCGCCCTCA GACGAGTCGG ATCTCCCTTT GGGCCGCCTC CCCGCATCGA TACCGTCGAC |
| | 4741 CTCGATCGAG ACCTAGAAAA ACATGGAGCA ATCACAAGTA GCAATACAGC AGCTACCAAT |
| | 4801 GCTGATTGTG CCTGGCTAGA AGCACAAGAG GAGGAGGAGG TGGGTTTTCC AGTCACACCT |
| | 4861 CAGGTACCTT TAAGACCAAT GACTTACAAG GCAGCTGTAG ATCTTAGCCA CTTTTTAAAA |
| | 4921 GAAAAGGGGG GACTGGAAGG GCTAATTCAC TCCCAACGAA GACAAGATAT CCTTGATCTG |
| | 4981 TGGATCTACC ACACACAAGG CTACTTCCCT GATTGGCAGA ACTACACACC AGGGCCAGGG |
| | 5041 ATCAGATATC CACTGACCTT TGGATGGTGC TACAAGCTAG TACCAGTTGA GCAAGAGAAG |
| | 5102 GTAGAAGAAG CCAATGAAGG AGAGAACACC CGCTTGTTAC ACCCTGTGAG CCTGCATGGG |
| | 5161 ATGGATGACC CGGAGAGAGA AGTATTAGAG TGGAGGTTTG ACAGCCGCCT AGCATTTCAT |
| | 5221 CACATGGCCC GAGAGCTGCA TCCGGACTGT ACTGGGTCTC TCTGGTTAGA CCAGATCTGA |
| | 5281 GCCTGGGAGC TCTCTGGCTA ACTAGGGAAC CCACTGCTTA AGCCTCAATA AAGCTTGCCT |
| | 5341 TGAGTGCTTC AAGTAGTGTG TGCCCGTCTG TTGTGTGACT CTGGTAACTA GAGATCCCTC |
| | 5401 AGACCCTTTT AGTCAGTGTG GAAAATCTCT AGCAGCATGT GAGCAAAAGG CCAGCAAAAG |
| | 5461 GCCAGGAACC GTAAAAAGGC CGCGTTGCTG GCGTTTTTCC ATAGGCTCCG CCCCCCTGAC |
| | 5521 GAGCATCACA AAAATCGACG CTCAAGTCAG AGGTGGCGAA ACCCGACAGG ACTATAAAGA |
| | 5581 TACCAGGCGT TTCCCCCTGG AAGCTCCCTC GTGCGCTCTC CTGTTCCGAC CCTGCCGCTT |
| | 5641 ACCGGATACC TGTCCGCCTT TCTCCCTTCG GGAAGCGTGG CGCTTTCTCA TAGCTCACGC |
| | 5701 TGTAGGTATC TCAGTTCGGT GTAGGTCGTT CGCTCCAAGC TGGGCTGTGT GCACGAACCC |
| | 5761 CCCGTTCAGC CCGACCGCTG CGCCTTATCC GGTAACTATC GTCTTGAGTC CAACCCGGTA |
| | 5821 AGACACGACT TATCGCCACT GGCAGCAGCC ACTGGTAACA GGATTAGCAG AGCGAGGTAT |
| | 5881 GTAGGCGGTG CTACAGAGTT CTTGAAGTGG TGGCCTAACT ACGGCTACAC TAGAAGAACA |
| | 5941 GTATTTGGTA TCTGCGCTCT GCTGAAGCCA GTTACCTTCG GAAAAAGAGT TGGTAGCTCT |
| | 6001 TGATCCGGCA AACAAACCAC CGCTGGTAGC GGTGGTTTTT TTGTTTGCAA GCAGCAGATT |
| | 6061 ACGCGCAGAA AAAAAGGATC TCAAGAAGAT CCTTTGATCT TTTCTACGGG GTCTGACGCT |
| | 6121 CAGTGGAACG AAAACTCACG TTAAGGGATT TTGGTCATGA GATTATCAAA AAGGATCTTC |
| | 6181 ACCTAGATCC TTTTAAATTA AAAATGAAGT TTTAAATCAA TCTAAAGTAT ATATGAGTAA |
| | 6241 ACTTGGTCTG ACAGTTACCA ATGCTTAATC AGTGAGGCAC CTATCTCAGC GATCTGTCTA |
| | 6301 TTTCGTTCAT CCATAGTTGC CTGACTCCCC GTCGTGTAGA TAACTACGAT ACGGGAGGGC |
| | 6361 TTACCATCTG GCCCCAGTGC TGCAATGATA CCGCGAGACC CACGCTCACC GGCTCCAGAT |
| | 6421 TTATCAGCAA TAAACCAGCC AGCCGGAAGG GCCGAGCGCA GAAGTGGTCC TGCAACTTTA |
| | 6481 TCCGCCTCCA TCCAGTCTAT TAATTGTTGC CGGGAAGCTA GAGTAAGTAG TTCGCCAGTT |
| | 6541 AATAGTTTGC GCAACGTTGT TGCCATTGCT ACAGGCATCG TGGTGTCACG CTCGTCGTTT |
| | 6601 GGTATGGCTT CATTCAGCTC CGGTTCCCAA CGATCAAGGC GAGTTACATG ATCCCCCATG |
| | 6661 TTGTGCAAAA AAGCGGTTAG CTCCTTCGGT CCTCCGATCG TTGTCAGAAG TAAGTTGGCC |
| | 6721 GCAGTGTTAT CACTCATGGT TATGGCAGCA CTGCATAATT CTCTTACTGT CATGCCATCC |
| | 6781 GTAAGATGCT TTTCTGTGAC TGGTGAGTAC TCAACCAAGT CATTCTGAGA ATAGTGTATG |
| | 6841 CGGCGACCGA GTTGCTCTTG CCCGGCGTCA ATACGGGATA ATACCGCGCC ACATAGCAGA |
| | 6901 ACTTTAAAAG TGCTCATCAT TGGAAAACGT TCTTCGGGGC GAAAACTCTC AAGGATCTTA |
| | 6961 CCGCTGTTGA GATCCAGTTC GATGTAACCC ACTCGTGCAC CCAACTGATC TTCAGCATCT |
| | 7021 TTTACTTTCA CCAGCGTTTC TGGGTGAGCA AAAACAGGAA GGCAAAATGC CGCAAAAAAG |
| | 7081 GGAATAAGGG CGACACGGAA ATGTTGAATA CTCATACTCT TCCTTTTTCA ATATTATTGA |
| | 7141 AGCATTTATC AGGGTTATTG TCTCATGAGC GGATACATAT TTGAATGTAT TTAGAAAAAT |
| | 7201 AAACAAATAG GGGTTCCGCG CACATTTCCC CGAAAAGTGC CACCTGAC |

//

pLL3.4

| LOCUS PLENTILOX | | 7969 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | misc_recomb | 4769..4802 |
| | | /note=“LoxP” |
| | promoter | 2632..3841 |
| | | /note=“UbC promoter” |
| | gene | 6976..7836 |
| | | /note=“AmpR” |
| | rep_origin | 6158..6831 |
| | | /note=“pUC” |
| | misc_recomb | 3931..3966 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 4857..5446 |
| | | /note=“WRE element” |
| | LTR | 5466..6155 |
| | | /note=“3′ SIN LTR” |
| | gene | 3993..4715 |
| | | /note=“EGFP” |

| | |
|---|---|
| BASE COUNT 1988 A | 1938 C 2150 G 1893 T 0 OTHER |
| ORIGIN - | |

| (SEQ ID NO:6) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTGATC TGGCCTCCGC |
| | 2641 GCCGGGTTTT GGCGCCTCCC GCGGGCGCCC CCCTCCTCAC GGCGAGCGCT GCCACGTCAG |
| | 2701 ACGAAGGGCG CAGGAGCGTC CTGATCCTTC CGCCCGGACG CTCAGGACAG CGGCCCGCTG |
| | 2761 CTCATAAGAC TCGGCCTTAG AACCCCAGTA TCAGCAGAAG GACATTTTAG GACGGGACTT |
| | 2821 GGGTGACTCT AGGGCACTGG TTTTCTTTCC AGAGAGCGGA ACAGGCGAGG AAAAGTAGTC |
| | 2881 CCTTCTCGGC GATTCTGCGG AGGGATCTCC GTGGGGCGGT GAACGCCGAT GATTATATAA |
| | 2941 GGACGCGCCG GGTGTGGCAC AGCTAGTTCC GTCGCAGCCG GGATTTGGGT CGCGGTTCTT |
| | 3001 GTTTGTGGAT CGCTGTGATC GTCACTTGGT GAGTAGCGGG CTGCTGGGCT GGCCGGGGCT |
| | 3061 TTCGTGGCCG CCGGGCCGCT CGGTGGGACG GAAGCGTGTG GAGAGACCGC CAAGGGCTGT |
| | 3121 AGTCTGGGTC CGCGAGCAAG GTTGCCCTGA ACTGGGGGTT GGGGGGAGCG CAGCAAAATG |
| | 3181 GCGGCTGTTC CCGAGTCTTG AATGGAAGAC GCTTGTGAGG CGGGCTGTGA GGTCGTTGAA |
| | 3241 ACAAGGTGGG GGGCATGGTG GGCGGCAAGA ACCCAAGGTC TTGAGGCCTT CGCTAATGCG |
| | 3301 GGAAAGCTCT TATTCGGGTG AGATGGGCTG GGGCACCATC TGGGGACCCT GACGTGAAGT |
| | 3361 TTGTCACTGA CTGGAGAACT CGGTTTGTCG TCTGTTGCGG GGGCGGCAGT TATGCGGTGC |
| | 3421 CGTTGGGCAG TGCACCCGTA CCTTTGGGAG CGCGCGCCCT CGTCGTGTCG TGACGTCACC |
| | 3481 CGTTCTGTTG GCTTATAATG CAGGGTGGGG CCACCTGCCG GTAGGTGTGC GGTAGGCTTT |
| | 3541 TCTCCGTCGC AGGACGCAGG GTTCGGGCCT AGGGTAGGCT CTCCTGAATC GACAGGCGCC |
| | 3601 GGACCTCTGG TGAGGGGAGG GATAAGTGAG GCGTCAGTTT CTTTGGTCGG TTTTATGTAC |
| | 3661 CTATCTTCTT AAGTAGCTGA AGCTCCGGTT TTGAACTATG CGCTCGGGGT TGGCGAGTGT |
| | 3721 GTTTTGTGAA GTTTTTTAGG CACCTTTTGA AATGTAATCA TTTGGGTCAA TATGTAATTT |
| | 3781 TCAGTGTTAG ACTAGTAAAT TGTCCGCTAA ATTCTGGCCG TTTTTGGCTT TTTTGTTAGA |
| | 3841 CGAAGCTAAC GCGCTAGCCG TTAATTAAGC CTCGAGGTCG ACGGTATCGA TAAGCTCGCT |
| | 3901 TCACGAGATT CCAGCAGGTC GAGGGACCTA ATAACTTCGT ATAGCATACA TTATACGAAG |
| | 3961 TTATATTAAG GGTTCCAAGC TTAAGCGGCC GCGCCACCAT GGTGAGCAAG GGCGAGGAGC |
| | 4021 TGTTCACCGG GGTGGTGCCC ATCCTGGTCG AGCTGGACGG CGACGTAAAC GGCCACAAGT |
| | 4081 TCAGCGTGTC CGGCGAGGGC GAGGGCGATG CCACCTACGG CAAGCTGACC CTGAAGTTCA |
| | 4141 TCTGCACCAC CGGCAAGCTG CCCGTGCCCT GGCCCACCCT CGTGACCACC CTGACCTACG |
| | 4201 GCGTGCAGTG CTTCAGCCGC TACCCCGACC ACATGAAGCA GCACGACTTC TTCAAGTCCG |
| | 4261 CCATGCCCGA AGGCTACGTC CAGGAGCGCA CCATCTTCTT CAAGGACGAC GGCAACTACA |
| | 4321 AGACCCGCGC CGAGGTGAAG TTCGAGGGCG ACACCCTGGT GAACCGCATC GAGCTGAAGG |
| | 4381 GCATCGACTT CAAGGAGGAC GGCAACATCC TGGGGCACAA GCTGGAGTAC AACTACAACA |
| | 4441 GCCACAACGT CTATATCATG GCCGACAAGC AGAAGAACGG CATCAAGGTG AACTTCAAGA |
| | 4501 TCCGCCACAA CATCGAGGAC GGCAGCGTGC AGCTCGCCGA CCACTACCAG CAGAACACCC |
| | 4561 CCATCGGCGA CGGCCCCGTG CTGCTGCCCG ACAACCACTA CCTGAGCACC CAGTCCGCCC |
| | 4621 TGAGCAAAGA CCCCAACGAG AAGCGCGATC ACATGGTCCT GCTGGAGTTC GTGACCGCCG |
| | 4681 CCGGGATCAC TCTCGGCATG GACGAGCTGT ACAAGATGCA TGCCCCGGGA TGGCGCGCCA |
| | 4741 TGGATCCGCG AATTCGTCGA GGGACCTAAT AACTTCGTAT AGCATACATT ATACGAAGTT |
| | 4801 ATACATGTTT AAGGGTTCCG GTTCCACTAG GTACAATTCG ATATCAAGCT TATCGATAAT |
| | 4861 CAACCTCTGG ATTACAAAAT TTGTGAAAGA TTGACTGGTA TTCTTAACTA TGTTGCTCCT |
| | 4921 TTTACGCTAT GTGGATACGC TGCTTTAATG CCTTTGTATC ATGCTATTGC TTCCCGTATG |
| | 4981 GCTTTCATTT TCTCCTCCTT GTATAAATCC TGGTTGCTGT CTCTTTATGA GGAGTTGTGG |
| | 5041 CCCGTTGTCA GGCAACGTGG CGTGGTGTGC ACTGTGTTTG CTGACGCAAC CCCCACTGGT |
| | 5101 TGGGGCATTG CCACCACCTG TCAGCTCCTT TCCGGGACTT TCGCTTTCCC CCTCCCTATT |
| | 5161 GCCACGGCGG AACTCATCGC CGCCTGCCTT GCCCGCTGCT GGACAGGGGC TCGGCTGTTG |
| | 5221 GGCACTGACA ATTCCGTGGT GTTGTCGGGG AAATCATCGT CCTTTCCTTG GCTGCTCGCC |
| | 5281 TGTGTTGCCA CCTGGATTCT GCGCGGGACG TCCTTCTGCT ACGTCCCTTC GGCCCTCAAT |
| | 5341 CCAGCGGACC TTCCTTCCCG CGGCCTGCTG CCGGCTCTGC GGCCTCTTCC GCGTCTTCGC |
| | 5401 CTTCGCCCTC AGACGAGTCG GATCTCCCTT TGGGCCGCCT CCCCGCATCG ATACCGTCGA |
| | 5461 CCTCGATCGA GACCTAGAAA AACATGGAGC AATCACAAGT AGCAATACAG CAGCTACCAA |
| | 5521 TGCTGATTGT GCCTGGCTAG AAGCACAAGA GGAGGAGGAG GTGGGTTTTC CAGTCACACC |
| | 5581 TCAGGTACCT TTAAGACCAA TGACTTACAA GGCAGCTGTA GATCTTAGCC ACTTTTTAAA |
| | 5641 AGAAAAGGGG GGACTGGAAG GGCTAATTCA CTCCCAACGA AGACAAGATA TCCTTGATCT |
| | 5701 GTGGATCTAC CACACACAAG GCTACTTCCC TGATTGGCAG AACTACACAC CAGGGCCAGG |
| | 5761 GATCAGATAT CCACTGACCT TTGGATGGTG CTACAAGCTA GTACCAGTTG AGCAAGAGAA |
| | 5821 GGTAGAAGAA GCCAATGAAG GAGAGAACAC CCGCTTGTTA CACCCTGTGA GCCTGCATGG |
| | 5881 GATGGATGAC CCGGAGAGAG AAGTATTAGA GTGGAGGTTT GACAGCCGCC TAGCATTTCA |
| | 5941 TCACATGGCC CGAGAGCTGC ATCCGGACTG TACTGGGTCT CTCTGGTTAG ACCAGATCTG |
| | 6001 AGCCTGGGAG CTCTCTGGCT AACTAGGGAA CCCACTGCTT AAGCCTCAAT AAAGCTTGCC |
| | 6061 TTGAGTGCTT CAAGTAGTGT GTGCCCGTCT GTTGTGTGAC TCTGGTAACT AGAGATCCCT |
| | 6121 CAGACCCTTT TAGTCAGTGT GGAAAATCTC TAGCAGCATG TGAGCAAAAG GCCAGCAAAA |
| | 6181 GGCCAGGAAC CGTAAAAAGG CCGCGTTGCT GGCGTTTTTC CATAGGCTCC GCCCCCCTGA |
| | 6241 CGAGCATCAC AAAAATCGAC GCTCAAGTCA GAGGTGGCGA AACCCGACAG GACTATAAAG |
| | 6301 ATACCAGGCG TTTCCCCCTG GAAGCTCCCT CGTGCGCTCT CCTGTTCCGA CCCTGCCGCT |
| | 6361 TACCGGATAC CTGTCCGCCT TTCTCCCTTC GGGAAGCGTG GCGCTTTCTC ATAGCTCACG |
| | 6421 CTGTAGGTAT CTCAGTTCGG TGTAGGTCGT TCGCTCCAAG CTGGGCTGTG TGCACGAACC |
| | 6481 CCCCGTTCAG CCCGACCGCT GCGCCTTATC CGGTAACTAT CGTCTTGAGT CCAACCCGGT |
| | 6541 AAGACACGAC TTATCGCCAC TGGCAGCAGC CACTGGTAAC AGGATTAGCA GAGCGAGGTA |
| | 6601 TGTAGGCGGT GCTACAGAGT TCTTGAAGTG GTGGCCTAAC TACGGCTACA CTAGAAGAAC |
| | 6661 AGTATTTGGT ATCTGCGCTC TGCTGAAGCC AGTTACCTTC GGAAAAAGAG TTGGTAGCTC |
| | 6721 TTGATCCGGC AAACAAACCA CCGCTGGTAG CGGTGGTTTT TTTGTTTGCA AGCAGCAGAT |
| | 6781 TACGCGCAGA AAAAAAGGAT CTCAAGAAGA TCCTTTGATC TTTTCTACGG GGTCTGACGC |
| | 6841 TCAGTGGAAC GAAAACTCAC GTTAAGGGAT TTTGGTCATG AGATTATCAA AAAGGATCTT |
| | 6901 CACCTAGATC CTTTTAAATT AAAAATGAAG TTTTAAATCA ATCTAAAGTA TATATGAGTA |
| | 6961 AACTTGGTCT GACAGTTACC AATGCTTAAT CAGTGAGGCA CCTATCTCAG CGATCTGTCT |
| | 7021 ATTTCGTTCA TCCATAGTTG CCTGACTCCC CGTCGTGTAG ATAACTACGA TACGGGAGGG |
| | 7081 CTTACCATCT GGCCCCAGTG CTGCAATGAT ACCGCGAGAC CCACGCTCAC CGGCTCCAGA |
| | 7141 TTTATCAGCA ATAAACCAGC CAGCCGGAAG GGCCGAGCGC AGAAGTGGTC CTGCAACTTT |
| | 7201 ATCCGCCTCC ATCCAGTCTA TTAATTGTTG CCGGGAAGCT AGAGTAAGTA GTTCGCCAGT |
| | 7261 TAATAGTTTG CGCAACGTTG TTGCCATTGC TACAGGCATC GTGGTGTCAC GCTCGTCGTT |
| | 7321 TGGTATGGCT TCATTCAGCT CCGGTTCCCA ACGATCAAGG CGAGTTACAT GATCCCCCAT |
| | 7381 GTTGTGCAAA AAAGCGGTTA GCTCCTTCGG TCCTCCGATC GTTGTCAGAA GTAAGTTGGC |
| | 7441 CGCAGTGTTA TCACTCATGG TTATGGCAGC ACTGCATAAT TCTCTTACTG TCATGCCATC |
| | 7501 CGTAAGATGC TTTTCTGTGA CTGGTGAGTA CTCAACCAAG TCATTCTGAG AATAGTGTAT |
| | 7561 GCGGCGACCG AGTTGCTCTT GCCCGGCGTC AATACGGGAT AATACCGCGC CACATAGCAG |
| | 7621 AACTTTAAAA GTGCTCATCA TTGGAAAACG TTCTTCGGGG CGAAAACTCT CAAGGATCTT |
| | 7681 ACCGCTGTTG AGATCCAGTT CGATGTAACC CACTCGTGCA CCCAACTGAT CTTCAGCATC |
| | 7741 TTTTACTTTC ACCAGCGTTT CTGGGTGAGC AAAAACAGGA AGGCAAAATG CCGCAAAAAA |
| | 7801 GGGAATAAGG GCGACACGGA AATGTTGAAT ACTCATACTC TTCCTTTTTC AATATTATTG |
| | 7861 AAGCATTTAT CAGGGTTATT GTCTCATGAG CGGATACATA TTTGAATGTA TTTAGAAAAA |
| | 7921 TAAACAAATA GGGGTTCCGC GCACATTTCC CCGAAAAGTG CCACCTGAC |

//

pLL3.5

| LOCUS PLL3.5.GB | | 7927 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | misc_recomb | 4727..4760 |
| | | /note=“LoxP” |
| | promoter | 2632..3841 |
| | | /note=“UbC promoter” |
| | gene | 6934..7794 |
| | | /note=“AmpR” |
| | rep_origin | 6116..6789 |
| | | /note=“pUC” |
| | misc_recomb | 3931..3966 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 4815..5404 |
| | | /note=“WRE element” |
| | LTR | 5424..6113 |
| | | /note=“3′ SIN LTR” |
| | gene | 3993..4673 |
| | | /note=“dsRed2” |

| | |
|---|---|
| BASE COUNT 1958 A | 1925 C 2151 G 1893 T 0 OTHER |
| ORIGIN - | |

| (SEQ ID NO:7) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCACTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTGATC TGGCCTCCGC |
| | 2641 GCCGGGTTTT GGCGCCTCCC GCGGGCGCCC CCCTCCTCAC GGCGAGCGCT GCCACGTCAG |
| | 2701 ACGAAGGGCG CAGGAGCGTC CTGATCCTTC CGCCCGGACG CTCAGGACAG CGGCCCGCTG |
| | 2761 CTCATAAGAC TCGGCCTTAG AACCCCAGTA TCAGCAGAAG GACATTTTAG GACGGGACTT |
| | 2821 GGGTGACTCT AGGGCACTGG TTTTCTTTCC AGAGAGCGGA ACAGGCGAGG AAAAGTAGTC |
| | 2881 CCTTCTCGGC GATTCTGCGG AGGGATCTCC GTGGGGCGGT GAACGCCGAT GATTATATAA |
| | 2941 GGACGCGCCG GGTGTGGCAC AGCTAGTTCC GTCGCAGCCG GGATTTGGGT CGCGGTTCTT |
| | 3001 GTTTGTGGAT CGCTGTGATC GTCACTTGGT GAGTAGCGGG CTGCTGGGCT GGCCGGGGCT |
| | 3061 TTCGTGGCCG CCGGGCCGCT CGGTGGGACG GAAGCGTGTG GAGAGACCGC CAAGGGCTGT |
| | 3121 AGTCTGGGTC CGCGAGCAAG GTTGCCCTGA ACTGGGGGTT GGGGGGAGCG CAGCAAAATG |
| | 3181 GCGGCTGTTC CCGAGTCTTG AATGGAAGAC GCTTGTGAGG CGGGCTGTGA GGTCGTTGAA |
| | 3241 ACAAGGTGGG GGGCATGGTG GGCGGCAAGA ACCCAAGGTC TTGAGGCCTT CGCTAATGCG |
| | 3301 GGAAAGCTCT TATTCGGGTG AGATGGGCTG GGGCACCATC TGGGGACCCT GACGTGAAGT |
| | 3361 TTGTCACTGA CTGGAGAACT CGGTTTGTCG TCTGTTGCGG GGGCGGCAGT TATGCGGTGC |
| | 3421 CGTTGGGCAG TGCACCCGTA CCTTTGGGAG CGCGCGCCCT CGTCGTGTCG TGACGTCACC |
| | 3481 CGTTCTGTTG GCTTATAATG CAGGGTGGGG CCACCTGCCG GTAGGTGTGC GGTAGGCTTT |
| | 3541 TCTCCGTCGC AGGACGCAGG GTTCGGGCCT AGGGTAGGCT CTCCTGAATC GACAGGCGCC |
| | 3601 GGACCTCTGG TGAGGGGAGG GATAAGTGAG GCGTCAGTTT CTTTGGTCGG TTTTATGTAC |
| | 3661 CTATCTTCTT AAGTAGCTGA AGCTCCGGTT TTGAACTATG CGCTCGGGGT TGGCGAGTGT |
| | 3721 GTTTTGTGAA GTTTTTTAGG CACCTTTTGA AATGTAATCA TTTGGGTCAA TATGTAATTT |
| | 3781 TCAGTGTTAG ACTAGTAAAT TGTCCGCTAA ATTCTGGCCG TTTTTGGCTT TTTTGTTAGA |
| | 3841 CGAAGCTAAC GCGCTAGCCG TTAATTAAGC CTCGAGGTCG ACGGTATCGA TAAGCTCGCT |
| | 3901 TCACGAGATT CCAGCAGGTC GAGGGACCTA ATAACTTCGT ATAGCATACA TTATACGAAG |
| | 3961 TTATATTAAG GGTTCCAAGC TTAAGCGGCC GCGCCACCAT GGCCTCCTCC GAGAACGTCA |
| | 4021 TCACCGAGTT CATGCGCTTC AAGGTGCGCA TGGAGGGCAC CGTGAACGGC CACGAGTTCG |
| | 4081 AGATCGAGGG CGAGGGCGAG GGCCGCCCCT ACGAGGGCCA CAACACCGTG AAGCTGAAGG |
| | 4141 TGACCAAGGG CGGCCCCCTG CCCTTCGCCT GGGACATCCT GTCCCCCCAG TTCCAGTACG |
| | 4201 GCTCCAAGGT GTACGTGAAG CACCCCGCCG ACATCCCCGA CTACAAGAAG CTGTCCTTCC |
| | 4261 CCGAGGGCTT CAAGTGGGAG CGCGTGATGA ACTTCGAGGA CGGCGGCGTG GCGACCGTGA |
| | 4321 CCCAGGACTC CTCCCTGCAG GACGGCTGCT TCATCTACAA GGTGAAGTTC ATCGGCGTGA |
| | 4381 ACTTCCCCTC CGACGGCCCC GTGATGCAGA AGAAGACCAT GGGCTGGGAG GCCTCCACCG |
| | 4441 AGCGCCTGTA CCCCCGCGAC GGCGTGCTGA AGGGCGAGAC CCACAAGGCC CTGAAGCTGA |
| | 4501 AGGACGGCGG CCACTACCTG GTGGAGTTCA AGTCCATCTA CATGGCCAAG AAGCCCGTGC |
| | 4561 AGCTGCCCGG CTACTACTAC GTGGACGCCA AGCTGGACAT CACCTCCCAC AACGAGGACT |
| | 4621 ACACCATCGT GGAGCAGTAC GAGCGCACCG AGGGCCGCCA CCACCTGTTC CTGATGCATG |
| | 4681 CCCCGGGATG GCGCGCCATG GATCCGCGAA TTCGTCGAGG GACCTAATAA CTTCGTATAG |
| | 4741 CATACATTAT ACGAAGTTAT ACATGTTTAA GGGTTCCGGT TCCACTAGGT ACAATTCGAT |
| | 4801 ATCAAGCTTA TCGATAATCA ACCTCTGGAT TACAAAATTT GTGAAAGATT GACTGGTATT |
| | 4861 CTTAACTATG TTGCTCCTTT TACGCTATGT GGATACGCTG CTTTAATGCC TTTGTATCAT |
| | 4921 GCTATTGCTT CCCGTATGGC TTTCATTTTC TCCTCCTTGT ATAAATCCTG GTTGCTGTCT |
| | 4981 CTTTATGAGG AGTTGTGGCC CGTTGTCAGG CAACGTGGCG TGGTGTGCAC TGTGTTTGCT |
| | 5041 GACGCAACCC CCACTGGTTG GGGCATTGCC ACCACCTGTC AGCTCCTTTC CGGGACTTTC |
| | 5101 GCTTTCCCCC TCCCTATTGC CACGGCGGAA CTCATCGCCG CCTGCCTTGC CCGCTGCTGG |
| | 5161 ACAGGGGCTC GGCTGTTGGG CACTGACAAT TCCGTGGTGT TGTCGGGGAA ATCATCGTCC |
| | 5221 TTTCCTTGGC TGCTCGCCTG TGTTGCCACC TGGATTCTGC GCGGGACGTC CTTCTGCTAC |
| | 5281 GTCCCTTCGG CCCTCAATCC AGCGGACCTT CCTTCCCGCG GCCTGCTGCC GGCTCTGCGG |
| | 5341 CCTCTTCCGC GTCTTCGCCT TCGCCCTCAG ACGAGTCGGA TCTCCCTTTG GGCCGCCTCC |
| | 5401 CCGCATCGAT ACCGTCGACC TCGATCGAGA CCTAGAAAAA CATGGAGCAA TCACAAGTAG |
| | 5461 CAATACAGCA GCTACCAATG CTGATTGTGC CTGGCTAGAA GCACAAGAGG AGGAGGAGGT |
| | 5521 GGGTTTTCCA GTCACACCTC AGGTACCTTT AAGACCAATG ACTTACAAGG CAGCTGTAGA |
| | 5581 TCTTAGCCAC TTTTTAAAAG AAAAGGGGGG ACTGGAAGGG CTAATTCACT CCCAACGAAG |
| | 5641 ACAAGATATC CTTGATCTGT GGATCTACCA CACACAAGGC TACTTCCCTG ATTGGCAGAA |
| | 5701 CTACACACCA GGGCCAGGGA TCAGATATCC ACTGACCTTT GGATGGTGCT ACAAGCTAGT |
| | 5761 ACCAGTTGAG CAAGAGAAGG TAGAAGAAGC CAATGAAGGA GAGAACACCC GCTTGTTACA |
| | 5821 CCCTGTGAGC CTGCATGGGA TGGATGACCC GGAGAGAGAA GTATTAGAGT GGAGGTTTGA |
| | 5881 CAGCCGCCTA GCATTTCATC ACATGGCCCG AGAGCTGCAT CCGGACTGTA CTGGGTCTCT |
| | 5941 CTGGTTAGAC CAGATCTGAG CCTGGGAGCT CTCTGGCTAA CTAGGGAACC CACTGCTTAA |
| | 6001 GCCTCAATAA AGCTTGCCTT GAGTGCTTCA AGTAGTGTGT GCCCGTCTGT TGTGTGACTC |
| | 6061 TGGTAACTAG AGATCCCTCA GACCCTTTTA GTCAGTGTGG AAAATCTCTA GCAGCATGTG |
| | 6121 AGCAAAAGGC CAGCAAAAGG CCAGGAACCG TAAAAAGGCC GCGTTGCTGG CGTTTTTCCA |
| | 6181 TAGGCTCCGC CCCCCTGACG AGCATCACAA AAATCGACGC TCAAGTCAGA GGTGGCGAAA |
| | 6241 CCCGACAGGA CTATAAAGAT ACCAGGCGTT TCCCCCTGGA AGCTCCCTCG TGCGCTCTCC |
| | 6301 TGTTCCGACC CTGCCGCTTA CCGGATACCT GTCCGCCTTT CTCCCTTCGG GAAGCGTGGC |
| | 6361 GCTTTCTCAT AGCTCACGCT GTAGGTATCT CAGTTCGGTG TAGGTCGTTC GCTCCAAGCT |
| | 6421 GGGCTGTGTG CACGAACCCC CCGTTCAGCC CGACCGCTGC GCCTTATCCG GTAACTATCG |
| | 6481 TCTTGAGTCC AACCCGGTAA GACACGACTT ATCGCCACTG GCAGCAGCCA CTGGTAACAG |
| | 6541 GATTAGCAGA GCGAGGTATG TAGGCGGTGC TACAGAGTTC TTGAAGTGGT GGCCTAACTA |
| | 6601 CGGCTACACT AGAAGAACAG TATTTGGTAT CTGCGCTCTG CTGAAGCCAG TTACCTTCGG |
| | 6661 AAAAAGAGTT GGTAGCTCTT GATCCGGCAA ACAAACCACC GCTGGTAGCG GTGGTTTTTT |
| | 6721 TGTTTGCAAG CAGCAGATTA CGCGCAGAAA AAAAGGATCT CAAGAAGATC CTTTGATCTT |
| | 6781 TTCTACGGGG TCTGACGCTC AGTGGAACGA AAACTCACGT TAAGGGATTT TGGTCATGAG |
| | 6841 ATTATCAAAA AGGATCTTCA CCTAGATCCT TTTAAATTAA AAATGAAGTT TTAAATCAAT |
| | 6901 CTAAAGTATA TATGAGTAAA CTTGGTCTGA CAGTTACCAA TGCTTAATCA GTGAGGCACC |
| | 6961 TATCTCAGCG ATCTGTCTAT TTCGTTCATC CATAGTTGCC TGACTCCCCG TCGTGTAGAT |
| | 7021 AACTACGATA CGGGAGGGCT TACCATCTGG CCCCAGTGCT GCAATGATAC CGCGAGACCC |
| | 7081 ACGCTCACCG GCTCCAGATT TATCAGCAAT AAACCAGCCA GCCGGAAGGG CCGAGCGCAG |
| | 7141 AAGTGGTCCT GCAACTTTAT CCGCCTCCAT CCAGTCTATT AATTGTTGCC GGGAAGCTAG |
| | 7201 AGTAAGTAGT TCGCCAGTTA ATAGTTTGCG CAACGTTGTT GCCATTGCTA CAGGCATCGT |
| | 7261 GGTGTCACGC TCGTCGTTTG GTATGGCTTC ATTCAGCTCC GGTTCCCAAC GATCAAGGCG |
| | 7321 AGTTACATGA TCCCCCATGT TGTGCAAAAA AGCGGTTAGC TCCTTCGGTC CTCCGATCGT |
| | 7381 TGTCAGAAGT AAGTTGGCCG CAGTGTTATC ACTCATGGTT ATGGCAGCAC TGCATAATTC |
| | 7441 TCTTACTGTC ATGCCATCCG TAAGATGCTT TTCTGTGACT GGTGAGTACT CAACCAAGTC |
| | 7501 ATTCTGAGAA TAGTGTATGC GGCGACCGAG TTGCTCTTGC CCGGCGTCAA TACGGGATAA |
| | 7561 TACCGCGCCA CATAGCAGAA CTTTAAAAGT GCTCATCATT GGAAAACGTT CTTCGGGGCG |
| | 7621 AAAACTCTCA AGGATCTTAC CGCTGTTGAG ATCCAGTTCG ATGTAACCCA CTCGTGCACC |
| | 7681 CAACTGATCT TCAGCATCTT TTACTTTCAC CAGCGTTTCT GGGTGAGCAA AAACAGGAAG |
| | 7741 GCAAAATGCC GCAAAAAAGG GAATAAGGGC GACACGGAAA TGTTGAATAC TCATACTCTT |
| | 7801 CCTTTTTCAA TATTATTGAA GCATTTATCA GGGTTATTGT CTCATGAGCG GATACATATT |
| | 7861 TGAATGTATT TAGAAAAATA AACAAATAGG GGTTCCGCGC ACATTTCCCC GAAAAGTGCC |
| | 7921 ACCTGAC |

//

pLL3.6

| LOCUS PLENTILOX | | 7350 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | promoter | 2799..3387 |
| | | /note=“CMV” |
| | gene | 6357..7217 |
| | | /note=“AmpR” |
| | rep_origin | 5539.. 6212 |
| | | /note=“pUC” |
| | misc_recomb | 2710..2745 |
| | | /note=“Lox 1” |
| | misc_recomb | 4150..4183 |
| | | /note=“LoxP” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 4238..4827 |
| | | /note=“WRE element” |
| | LTR | 4847..5536 |
| | | /note=“3′ SIN LTR” |
| | frag | 2772..4130 |
| | | /note=“1 to 1359 of Untitled1” |
| | frag | 2772..2798 |
| | | /note=“4705 to 4731 of pEGFP-C1” |
| | frag | 2799..4127 |
| | | /note=“1 to 1329 of pEGFP-C1” |
| | gene | 3404..4127 |
| | | /note=“EGFP” |

| | |
|---|---|
| BASE COUNT 1939 A | 1795 C 1862 G 1754 T 0 OTHER |
| ORIGIN - | |

| (SEQ ID NO:8) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG ACGGTTAACG CGCTAGCCGT |
| | 2641 TAATTAAGCC TCGAGGTCGA CGGTATCGAT AAGCTCGCTT CACGAGATTC CAGCAGGTCG |
| | 2701 AGGGACCTAA TAACTTCGTA TAGCATACAT TATACGAAGT TATATTAAGG GTTCCAAGCT |
| | 2761 TAAGCGGCCG CGTGGATAAC CGTATTACCG CCATGCATTA GTTATTAATA GTAATCAATT |
| | 2821 ACGGGGTCAT TAGTTCATAG CCCATATATG GAGTTCCGCG TTACATAACT TACGGTAAAT |
| | 2881 GGCCCGCCTG GCTGACCGCC CAACGACCCC CGCCCATTGA CGTCAATAAT GACGTATGTT |
| | 2941 CCCATAGTAA CGCCAATAGG GACTTTCCAT TGACGTCAAT GGGTGGAGTA TTTACGGTAA |
| | 3001 ACTGCCCACT TGGCAGTACA TCAAGTGTAT CATATGCCAA GTACGCCCCC TATTGACGTC |
| | 3061 AATGACGGTA AATGGCCCGC CTGGCATTAT GCCCAGTACA TGACCTTATG GGACTTTCCT |
| | 3121 ACTTGGCAGT ACATCTACGT ATTAGTCATC GCTATTACCA TGGTGATGCG GTTTTGGCAG |
| | 3181 TACATCAATG GGCGTGGATA GCGGTTTGAC TCACGGGGAT TTCCAAGTCT CCACCCCATT |
| | 3241 GACGTCAATG GGAGTTTGTT TTGGCACCAA AATCAACGGG ACTTTCCAAA ATGTCGTAAC |
| | 3301 AACTCCGCCC CATTGACGCA AATGGGCGGT AGGCGTGTAC GGTGGGAGGT CTATATAAGC |
| | 3361 AGAGCTGGTT TAGTGAACCG TCAGATCCGC TAGCGCTACC GGTCGCCACC ATGGTGAGCA |
| | 3421 AGGGCGAGGA GCTGTTCACC GGGGTGGTGC CCATCCTGGT CGAGCTGGAC GGCGACGTAA |
| | 3481 ACGGCCACAA GTTCAGCGTG TCCGGCGAGG GCGAGGGCGA TGCCACCTAC GGCAAGCTGA |
| | 3541 CCCTGAAGTT CATCTGCACC ACCGGCAAGC TGCCCGTGCC CTGGCCCACC CTCGTGACCA |
| | 3601 CCCTGACCTA CGGCGTGCAG TGCTTCAGCC GCTACCCCGA CCACATGAAG CAGCACGACT |
| | 3661 TCTTCAAGTC CGCCATGCCC GAAGGCTACG TCCAGGAGCG CACCATCTTC TTCAAGGACG |
| | 3721 ACGGCAACTA CAAGACCCGC GCCGAGGTGA AGTTCGAGGG CGACACCCTG GTGAACCGCA |
| | 3781 TCGAGCTGAA GGGCATCGAC TTCAAGGAGG ACGGCAACAT CCTGGGGCAC AAGCTGGAGT |
| | 3841 ACAACTACAA CAGCCACAAC GTCTATATCA TGGCCGACAA GCAGAAGAAC GGCATCAAGG |
| | 3901 TGAACTTCAA GATCCGCCAC AACATCGAGG ACGGCAGCGT GCAGCTCGCC GACCACTACC |
| | 3961 AGCAGAACAC CCCCATCGGC GACGGCCCCG TGCTGCTGCC CGACAACCAC TACCTGAGCA |
| | 4021 CCCAGTCCGC CCTGAGCAAA GACCCCAACG AGAAGCGCGA TCACATGGTC CTGCTGGAGT |
| | 4081 TCGTGACCGC CGCCGGGATC ACTCTCGGCA TGGACGAGCT GTACAAGTAG GAATTCGTCG |
| | 4141 AGGGACCTAA TAACTTCGTA TAGCATACAT TATACGAAGT TATACATGTT TAAGGGTTCC |
| | 4201 GGTTCCACTA GGTACAATTC GATATCAAGC TTATCGATAA TCAACCTCTG GATTACAAAA |
| | 4261 TTTGTGAAAG ATTGACTGGT ATTCTTAACT ATGTTGCTCC TTTTACGCTA TGTGGATACG |
| | 4321 CTGCTTTAAT GCCTTTGTAT CATGCTATTG CTTCCCGTAT GGCTTTCATT TTCTCCTCCT |
| | 4381 TGTATAAATC CTGGTTGCTG TCTCTTTATG AGGAGTTGTG GCCCGTTGTC AGGCAACGTG |
| | 4441 GCGTGGTGTG CACTGTGTTT GCTGACGCAA CCCCCACTGG TTGGGGCATT GCCACCACCT |
| | 4501 GTCAGCTCCT TTCCGGGACT TTCGCTTTCC CCCTCCCTAT TGCCACGGCG GAACTCATCG |
| | 4561 CCGCCTGCCT TGCCCGCTGC TGGACAGGGG CTCGGCTGTT GGGCACTGAC AATTCCGTGG |
| | 4621 TGTTGTCGGG GAAATCATCG TCCTTTCCTT GGCTGCTCGC CTGTGTTGCC ACCTGGATTC |
| | 4681 TGCGCGGGAC GTCCTTCTGC TACGTCCCTT CGGCCCTCAA TCCAGCGGAC CTTCCTTCCC |
| | 4741 GCGGCCTGCT GCCGGCTCTG CGGCCTCTTC CGCGTCTTCG CCTTCGCCCT CAGACGAGTC |
| | 4801 GGATCTCCCT TTGGGCCGCC TCCCCGCATC GATACCGTCG ACCTCGATCG AGACCTAGAA |
| | 4861 AAACATGGAG CAATCACAAG TAGCAATACA GCAGCTACCA ATGCTGATTG TGCCTGGCTA |
| | 4921 GAAGCACAAG AGGAGGAGGA GGTGGGTTTT CCAGTCACAC CTCAGGTACC TTTAAGACCA |
| | 4981 ATGACTTACA AGGCAGCTGT AGATCTTAGC CACTTTTTAA AAGAAAAGGG GGGACTGGAA |
| | 5041 GGGCTAATTC ACTCCCAACG AAGACAAGAT ATCCTTGATC TGTGGATCTA CCACACACAA |
| | 5101 GGCTACTTCC CTGATTGGCA GAACTACACA CCAGGGCCAG GGATCAGATA TCCACTGACC |
| | 5161 TTTGGATGGT GCTACAAGCT AGTACCAGTT GAGCAAGAGA AGGTAGAAGA AGCCAATGAA |
| | 5221 GGAGAGAACA CCCGCTTGTT ACACCCTGTG AGCCTGCATG GGATGGATGA CCCGGAGAGA |
| | 5281 GAAGTATTAG AGTGGAGGTT TGACAGCCGC CTAGCATTTC ATCACATGGC CCGAGAGCTG |
| | 5341 CATCCGGACT GTACTGGGTC TCTCTGGTTA GACCAGATCT GAGCCTGGGA GCTCTCTGGC |
| | 5401 TAACTAGGGA ACCCACTGCT TAAGCCTCAA TAAAGCTTGC CTTGAGTGCT TCAAGTAGTG |
| | 5461 TGTGCCCGTC TGTTGTGTGA CTCTGGTAAC TAGAGATCCC TCAGACCCTT TTAGTCAGTG |
| | 5521 TGGAAAATCT CTAGCAGCAT GTGAGCAAAA GGCCAGCAAA AGGCCAGGAA CCGTAAAAAG |
| | 5581 GCCGCGTTGC TGGCGTTTTT CCATAGGCTC CGCCCCCCTG ACGAGCATCA CAAAAATCGA |
| | 5641 CGCTCAAGTC AGAGGTGGCG AAACCCGACA GGACTATAAA GATACCAGGC GTTTCCCCCT |
| | 5701 GGAAGCTCCC TCGTGCGCTC TCCTGTTCCG ACCCTGCCGC TTACCGGATA CCTGTCCGCC |
| | 5761 TTTCTCCCTT CGGGAAGCGT GGCGCTTTCT CATAGCTCAC GCTGTAGGTA TCTCAGTTCG |
| | 5821 GTGTAGGTCG TTCGCTCCAA GCTGGGCTGT GTGCACGAAC CCCCCGTTCA GCCCGACCGC |
| | 5881 TGCGCCTTAT CCGGTAACTA TCGTCTTGAG TCCAACCCGG TAAGACACGA CTTATCGCCA |
| | 5941 CTGGCAGCAG CCACTGGTAA CAGGATTAGC AGAGCGAGGT ATGTAGGCGG TGCTACAGAG |
| | 6001 TTCTTGAAGT GGTGGCCTAA CTACGGCTAC ACTAGAAGAA CAGTATTTGG TATCTGCGCT |
| | 6061 CTGCTGAAGC CAGTTACCTT CGGAAAAAGA GTTGGTAGCT CTTGATCCGG CAAACAAACC |
| | 6121 ACCGCTGGTA GCGGTGGTTT TTTTGTTTGC AAGCAGCAGA TTACGCGCAG AAAAAAAGGA |
| | 6181 TCTCAAGAAG ATCCTTTGAT CTTTTCTACG GGGTCTGACG CTCAGTGGAA CGAAAACTCA |
| | 6241 CGTTAAGGGA TTTTGGTCAT GAGATTATCA AAAAGGATCT TCACCTAGAT CCTTTTAAAT |
| | 6301 TAAAAATGAA GTTTTAAATC AATCTAAAGT ATATATGAGT AAACTTGGTC TGACAGTTAC |
| | 6361 CAATGCTTAA TCAGTGAGGC ACCTATCTCA GCGATCTGTC TATTTCGTTC ATCCATAGTT |
| | 6421 GCCTGACTCC CCGTCGTGTA GATAACTACG ATACGGGAGG GCTTACCATC TGGCCCCAGT |
| | 6481 GCTGCAATGA TACCGCGAGA CCCACGCTCA CCGGCTCCAG ATTTATCAGC AATAAACCAG |
| | 6541 CCAGCCGGAA GGGCCGAGCG CAGAAGTGGT CCTGCAACTT TATCCGCCTC CATCCAGTCT |
| | 6601 ATTAATTGTT GCCGGGAAGC TAGAGTAAGT AGTTCGCCAG TTAATAGTTT GCGCAACGTT |
| | 6661 GTTGCCATTG CTACAGGCAT CGTGGTGTCA CGCTCGTCGT TTGGTATGGC TTCATTCAGC |
| | 6721 TCCGGTTCCC AACGATCAAG GCGAGTTACA TGATCCCCCA TGTTGTGCAA AAAAGCGGTT |
| | 6781 AGCTCCTTCG GTCCTCCGAT CGTTGTCAGA AGTAAGTTGG CCGCAGTGTT ATCACTCATG |
| | 6841 GTTATGGCAG CACTGCATAA TTCTCTTACT GTCATGCCAT CCGTAAGATG CTTTTCTGTG |
| | 6901 ACTGGTGAGT ACTCAACCAA GTCATTCTGA GAATAGTGTA TGCGGCGACC GAGTTGCTCT |
| | 6961 TGCCCGGCGT CAATACGGGA TAATACCGCG CCACATAGCA GAACTTTAAA AGTGCTCATC |
| | 7021 ATTGGAAAAC GTTCTTCGGG GCGAAAACTC TCAAGGATCT TACCGCTGTT GAGATCCAGT |
| | 7081 TCGATGTAAC CCACTCGTGC ACCCAACTGA TCTTCAGCAT CTTTTACTTT CACCAGCGTT |
| | 7141 TCTGGGTGAG CAAAAACAGG AAGGCAAAAT GCCGCAAAAA AGGGAATAAG GGCGACACGG |
| | 7201 AAATGTTGAA TACTCATACT CTTCCTTTTT CAATATTATT GAAGCATTTA TCAGGGTTAT |
| | 7261 TGTCTCATGA GCGGATACAT ATTTGAATGT ATTTAGAAAA ATAAACAAAT AGGGGTTCCG |
| | 7321 CGCACATTTC CCCGAAAAGT GCCACCTGAC |

//

pLL3.7

| LOCUS PLL3.7.GB | | 7650 BP DS-DNA CIRCULAR SYN 23-JAN-2002 |
|---|---|---|
| DEFINITION - | | |
| ACCESSION - | | |
| KEYWORDS - | | |
| SOURCE - | | |
| FEATURES | | Location/Qualifiers |
| | promoter | 212..816 |
| | | /note=“CMV promoter/enhancer 1” |
| | misc_recomb | 4450..4483 |
| | | /note=“Lox 2” |
| | promoter | 3099..3687 |
| | | /note=“CMV” |
| | gene | 6657..7517 |
| | | /note=“AmpR” |
| | rep_origin | 5839..6512 |
| | | /note=“pUC” |
| | misc_recomb | 3010..3045 |
| | | /note=“Lox 1” |
| | LTR | 835..1509 |
| | | /note=“5′ HIV R-U5-del gag (HIV NL4-3/454-1126)” |
| | misc_feature | 1539..2396 |
| | | /note=“HIV RRE (HIV NL4-3/7622-8459)” |
| | misc_feature | 2422..2599 |
| | | /note=“HIV Flap” |
| | misc_feature | 4538..5127 |
| | | /note=“WRE element” |
| | LTR | 5147..5836 |
| | | /note=“3′ SIN LTR” |
| | frag | 3072..4430 |
| | | /note=“1 to 1359 of Untitled1” |
| | frag | 3072..3098 |
| | | /note=“4705 to 4731 of pEGFP-C1” |
| | frag | 3099..4427 |
| | | /note=“1 to 1329 of pEGFP-C1” |
| | gene | 3704..4427 |
| | | /note=“EGFP” |
| | frag | 2617..2950 |
| | | /note=“1 to 334 of Untitled2” |
| | frag | 2622..2935 |
| | | /note=“1 to 314 of mouseu6” |
| | source | 2622..>2935 |
| | | /organism=“Mus musculus” |
| | | /db_xref=“taxon:10090” |
| | | /clone=“pmU6-52BE [Split]” |
| | promoter | 2622.. >2935 |
| | | /note=“U6 Promoter [Split]” |
| | misc_feature | 2648..2658 |
| | | /note=“pot. SPI binding site” |
| | misc_feature | 2692..2701 |
| | | /note=“pot. SPI binding site” |
| | misc_feature | 2707..2714 |
| | | /note=“pot. enhancer” |
| | promoter | 2869..2888 |
| | | /note=“pot. promoter region; sequence homologous to |
| PSE or | | |
| | | element ′B′” |
| | promoter | 2906..2911 |
| | | /note=“put. TATA-box” |

| | |
|---|---|
| BASE COUNT 2032 A | 1861 C 1917 G 1840 T 0 OTHER |
| ORIGIN - | |

| (SEQ ID NO:9) | |
|---|---|
| | 1 GTCGACGGAT CGGGAGATCT CCCGATCCCC TATGGTGCAC TCTCAGTACA ATCTGCTCTG |
| | 61 ATGCCGCATA GTTAAGCCAG TATCTGCTCC CTGCTTGTGT GTTGGAGGTC GCTGAGTAGT |
| | 121 GCGCGAGCAA AATTTAAGCT ACAACAAGGC AAGGCTTGAC CGACAATTGC ATGAAGAATC |
| | 181 TGCTTAGGGT TAGGCGTTTT GCGCTGCTTC GCGATGTACG GGCCAGATAT ACGCGTTGAC |
| | 241 ATTGATTATT GACTAGTTAT TAATAGTAAT CAATTACGGG GTCATTAGTT CATAGCCCAT |
| | 301 ATATGGAGTT CCGCGTTACA TAACTTACGG TAAATGGCCC GCCTGGCTGA CCGCCCAACG |
| | 361 ACCCCCGCCC ATTGACGTCA ATAATGACGT ATGTTCCCAT AGTAACGCCA ATAGGGACTT |
| | 421 TCCATTGACG TCAATGGGTG GAGTATTTAC GGTAAACTGC CCACTTGGCA GTACATCAAG |
| | 481 TGTATCATAT GCCAAGTACG CCCCCTATTG ACGTCAATGA CGGTAAATGG CCCGCCTGGC |
| | 541 ATTATGCCCA GTACATGACC TTATGGGACT TTCCTACTTG GCAGTACATC TACGTATTAG |
| | 601 TCATCGCTAT TACCATGGTG ATGCGGTTTT GGCAGTACAT CAATGGGCGT GGATAGCGGT |
| | 661 TTGACTCACG GGGATTTCCA AGTCTCCACC CCATTGACGT CAATGGGAGT TTGTTTTGGC |
| | 721 ACCAAAATCA ACGGGACTTT CCAAAATGTC GTAACAACTC CGCCCCATTG ACGCAAATGG |
| | 781 GCGGTAGGCG TGTACGGTGG GAGGTCTATA TAAGCAGCGC GTTTTGCCTG TACTGGGTCT |
| | 841 CTCTGGTTAG ACCAGATCTG AGCCTGGGAG CTCTCTGGCT AACTAGGGAA CCCACTGCTT |
| | 901 AAGCCTCAAT AAAGCTTGCC TTGAGTGCTT CAAGTAGTGT GTGCCCGTCT GTTGTGTGAC |
| | 961 TCTGGTAACT AGAGATCCCT CAGACCCTTT TAGTCAGTGT GGAAAATCTC TAGCAGTGGC |
| | 1021 GCCCGAACAG GGACTTGAAA GCGAAAGGGA AACCAGAGGA GCTCTCTCGA CGCAGGACTC |
| | 1081 GGCTTGCTGA AGCGCGCACG GCAAGAGGCG AGGGGCGGCG ACTGGTGAGT ACGCCAAAAA |
| | 1141 TTTTGACTAG CGGAGGCTAG AAGGAGAGAG ATGGGTGCGA GAGCGTCAGT ATTAAGCGGG |
| | 1201 GGAGAATTAG ATCGCGATGG GAAAAAATTC GGTTAAGGCC AGGGGGAAAG AAAAAATATA |
| | 1261 AATTAAAACA TATAGTATGG GCAAGCAGGG AGCTAGAACG ATTCGCAGTT AATCCTGGCC |
| | 1321 TGTTAGAAAC ATCAGAAGGC TGTAGACAAA TACTGGGACA GCTACAACCA TCCCTTCAGA |
| | 1381 CAGGATCAGA AGAACTTAGA TCATTATATA ATACAGTAGC AACCCTCTAT TGTGTGCATC |
| | 1441 AAAGGATAGA GATAAAAGAC ACCAAGGAAG CTTTAGACAA GATAGAGGAA GAGCAAAACA |
| | 1501 AAAGTAAGAC CACCGCACAG CAAGCGGCCG GCCGCGCTGA TCTTCAGACC TGGAGGAGGA |
| | 1561 GATATGAGGG ACAATTGGAG AAGTGAATTA TATAAATATA AAGTAGTAAA AATTGAACCA |
| | 1621 TTAGGAGTAG CACCCACCAA GGCAAAGAGA AGAGTGGTGC AGAGAGAAAA AAGAGCAGTG |
| | 1681 GGAATAGGAG CTTTGTTCCT TGGGTTCTTG GGAGCAGCAG GAAGCACTAT GGGCGCAGCG |
| | 1741 TCAATGACGC TGACGGTACA GGCCAGACAA TTATTGTCTG GTATAGTGCA GCAGCAGAAC |
| | 1801 AATTTGCTGA GGGCTATTGA GGCGCAACAG CATCTGTTGC AACTCACAGT CTGGGGCATC |
| | 1861 AAGCAGCTCC AGGCAAGAAT CCTGGCTGTG GAAAGATACC TAAAGGATCA ACAGCTCCTG |
| | 1921 GGGATTTGGG GTTGCTCTGG AAAACTCATT TGCACCACTG CTGTGCCTTG GAATGCTAGT |
| | 1981 TGGAGTAATA AATCTCTGGA ACAGATTTGG AATCACACGA CCTGGATGGA GTGGGACAGA |
| | 2041 GAAATTAACA ATTACACAAG CTTAATACAC TCCTTAATTG AAGAATCGCA AAACCAGCAA |
| | 2101 GAAAAGAATG AACAAGAATT ATTGGAATTA GATAAATGGG CAAGTTTGTG GAATTGGTTT |
| | 2161 AACATAACAA ATTGGCTGTG GTATATAAAA TTATTCATAA TGATAGTAGG AGGCTTGGTA |
| | 2221 GGTTTAAGAA TAGTTTTTGC TGTACTTTCT ATAGTGAATA GAGTTAGGCA GGGATATTCA |
| | 2281 CCATTATCGT TTCAGACCCA CCTCCCAACC CCGAGGGGAC CCGACAGGCC CGAAGGAATA |
| | 2341 GAAGAAGAAG GTGGAGAGAG AGACAGAGAC AGATCCATTC GATTAGTGAA CGGATCGGCA |
| | 2401 CTGCGTGCGC CAATTCTGCA GACAAATGGC AGTATTCATC CACAATTTTA AAAGAAAAGG |
| | 2461 GGGGATTGGG GGGTACAGTG CAGGGGAAAG AATAGTAGAC ATAATAGCAA CAGACATACA |
| | 2521 AACTAAAGAA TTACAAAAAC AAATTACAAA AATTCAAAAT TTTCGGGTTT ATTACAGGGA |
| | 2581 CAGCAGAGAT CCAGTTTGGT TAGTACCGGG CCCGCTCTAG AGATCCGACG CCGCCATCTC |
| | 2641 TAGGCCCGCG CCGGCCCCCT CGCACAGACT TGTGGGAGAA GCTCGGCTAC TCCCCTGCCC |
| | 2701 CGGTTAATTT GCATATAATA TTTCCTAGTA ACTATAGAGG CTTAATGTGC GATAAAAGAC |
| | 2761 AGATAATCTG TTCTTTTTAA TACTAGCTAC ATTTTACATG ATAGGCTTGG ATTTCTATAA |
| | 2821 GAGATACAAA TACTAAATTA TTATTTTAAA AAACAGCACA AAAGGAAACT CACCCTAACT |
| | 2881 GTAAAGTAAT TGTGTGTTTT GAGACTATAA ATATCCCTTG GAGAAAAGCC TTGTTAACGC |
| | 2941 GCGGTGACCC TCGAGGTCGA CGGTATCGAT AAGCTCGCTT CACGAGATTC CAGCAGGTCG |
| | 3001 AGGGACCTAA TAACTTCGTA TAGCATACAT TATACGAAGT TATATTAAGG GTTCCAAGCT |
| | 3061 TAAGCGGCCG CGTGGATAAC CGTATTACCG CCATGCATTA GTTATTAATA GTAATCAATT |
| | 3121 ACGGGGTCAT TAGTTCATAG CCCATATATG GAGTTCCGCG TTACATAACT TACGGTAAAT |
| | 3181 GGCCCGCCTG GCTGACCGCC CAACGACCCC CGCCCATTGA CGTCAATAAT GACGTATGTT |
| | 3241 CCCATAGTAA CGCCAATAGG GACTTTCCAT TGACGTCAAT GGGTGGAGTA TTTACGGTAA |
| | 3301 ACTGCCCACT TGGCAGTACA TCAAGTGTAT CATATGCCAA GTACGCCCCC TATTGACGTC |
| | 3361 AATGACGGTA AATGGCCCGC CTGGCATTAT GCCCAGTACA TGACCTTATG GGACTTTCCT |
| | 3421 ACTTGGCAGT ACATCTACGT ATTAGTCATC GCTATTACCA TGGTGATGCG GTTTTGGCAG |
| | 3481 TACATCAATG GGCGTGGATA GCGGTTTGAC TCACGGGGAT TTCCAAGTCT CCACCCCATT |
| | 3541 GACGTCAATG GGAGTTTGTT TTGGCACCAA AATCAACGGG ACTTTCCAAA ATGTCGTAAC |
| | 3601 AACTCCGCCC CATTGACGCA AATGGGCGGT AGGCGTGTAC GGTGGGAGGT CTATATAAGC |
| | 3661 AGAGCTGGTT TAGTGAACCG TCAGATCCGC TAGCGCTACC GGTCGCCACC ATCGTGAGCA |
| | 3721 AGGGCGAGGA GCTGTTCACC GGGGTGGTGC CCATCCTGGT CGAGCTGGAC GGCGACGTAA |
| | 3781 ACGGCCACAA GTTCAGCGTG TCCGGCGAGG GCGAGGGCGA TGCCACCTAC GGCAAGCTGA |
| | 3841 CCCTGAAGTT CATCTGCACC ACCGGCAAGC TGCCCGTGCC CTGGCCCACC CTCGTGACCA |
| | 3901 CCCTGACCTA CGGCGTGCAG TGCTTCAGCC GCTACCCCGA CCACATGAAG CAGCACGACT |
| | 3961 TCTTCAAGTC CGCCATGCCC GAAGGCTACG TCCAGGAGCG CACCATCTTC TTCAAGGACG |
| | 4021 ACGGCAACTA CAAGACCCGC GCCGAGGTGA AGTTCGAGGG CGACACCCTG GTGAACCGCA |
| | 4081 TCGAGCTGAA GGGCATCGAC TTCAAGGAGG ACGGCAACAT CCTGGGGCAC AAGCTGGAGT |
| | 4141 ACAACTACAA CAGCCACAAC GTCTATATCA TGGCCGACAA GCAGAAGAAC GGCATCAAGG |
| | 4201 TGAACTTCAA GATCCGCCAC AACATCGAGG ACGGCAGCGT GCAGCTCGCC GACCACTACC |
| | 4261 AGCAGAACAC CCCCATCGGC GACGGCCCCG TGCTGCTGCC CGACAACCAC TACCTGAGCA |
| | 4321 CCCAGTCCGC CCTGAGCAAA GACCCCAACG AGAAGCGCGA TCACATGGTC CTGCTGGAGT |
| | 4381 TCGTGACCGC CGCCGGGATC ACTCTCGGCA TGGACGAGCT GTACAAGTAG GAATTCGTCG |
| | 4441 AGGGACCTAA TAACTTCGTA TAGCATACAT TATACGAAGT TATACATGTT TAAGGGTTCC |
| | 4501 GGTTCCACTA GGTACAATTC GATATCAAGC TTATCGATAA TCAACCTCTG GATTACAAAA |
| | 4561 TTTGTGAAAG ATTGACTGGT ATTCTTAACT ATGTTGCTCC TTTTACGCTA TGTGGATACG |
| | 4621 CTGCTTTAAT GCCTTTGTAT CATGCTATTG CTTCCCGTAT GGCTTTCATT TTCTCCTCCT |
| | 4681 TGTATAAATC CTGGTTGCTG TCTCTTTATG AGGAGTTGTG GCCCGTTGTC AGGCAACGTG |
| | 4741 GCGTGGTGTG CACTGTGTTT GCTGACGCAA CCCCCACTGG TTGGGGCATT GCCACCACCT |
| | 4801 GTCAGCTCCT TTCCGGGACT TTCGCTTTCC CCCTCCCTAT TGCCACGGCG GAACTCATCG |
| | 4861 CCGCCTGCCT TGCCCGCTGC TGGACAGGGG CTCGGCTGTT GGGCACTGAC AATTCCGTGG |
| | 4921 TGTTGTCGGG GAAATCATCG TCCTTTCCTT GGCTGCTCGC CTGTGTTGCC ACCTGGATTC |
| | 4981 TGCGCGGGAC GTCCTTCTGC TACGTCCCTT CGGCCCTCAA TCCAGCGGAC CTTCCTTCCC |
| | 5041 GCGGCCTGCT GCCGGCTCTG CGGCCTCTTC CGCGTCTTCG CCTTCGCCCT CAGACGAGTC |
| | 5101 GGATCTCCCT TTGGGCCGCC TCCCCGCATC GATACCGTCG ACCTCGATCG AGACCTAGAA |
| | 5161 AAACATGGAG CAATCACAAG TAGCAATACA GCAGCTACCA ATGCTGATTG TGCCTGGCTA |
| | 5221 GAAGCACAAG AGGAGGAGGA GGTGGGTTTT CCAGTCACAC CTCAGGTACC TTTAAGACCA |
| | 5281 ATGACTTACA AGGCAGCTGT AGATCTTAGC CACTTTTTAA AAGAAAAGGG GGGACTGGAA |
| | 5341 GGGCTAATTC ACTCCCAACG AAGACAAGAT ATCCTTGATC TGTGGATCTA CCACACACAA |
| | 5401 GGCTACTTCC CTGATTGGCA GAACTACACA CCAGGGCCAG GGATCAGATA TCCACTGACC |
| | 5461 TTTGGATGGT GCTACAAGCT AGTACCAGTT GAGCAAGAGA AGGTAGAAGA AGCCAATGAA |
| | 5521 GGAGAGAACA CCCGCTTGTT ACACCCTGTG AGCCTGCATG GGATGGATGA CCCGGAGAGA |
| | 5581 GAAGTATTAG AGTGGAGGTT TGACAGCCGC CTAGCATTTC ATCACATGGC CCGAGAGCTG |
| | 5641 CATCCGGACT GTACTGGGTC TCTCTGGTTA GACCAGATCT GAGCCTGGGA GCTCTCTGGC |
| | 5701 TAACTAGGGA ACCCACTGCT TAAGCCTCAA TAAAGCTTGC CTTGAGTGCT TCAAGTAGTG |
| | 5761 TGTGCCCGTC TGTTGTGTGA CTCTGGTAAC TAGAGATCCC TCAGACCCTT TTAGTCAGTG |
| | 5821 TGGAAAATCT CTAGCAGCAT GTGAGCAAAA GGCCAGCAAA AGGCCAGGAA CCGTAAAAAG |
| | 5881 GCCGCGTTGC TGGCGTTTTT CCATAGGCTC CGCCCCCCTG ACGAGCATCA CAAAAATCGA |
| | 5941 CGCTCAAGTC AGAGGTGGCG AAACCCGACA GGACTATAAA GATACCAGGC GTTTCCCCCT |
| | 6001 GGAAGCTCCC TCGTGCGCTC TCCTGTTCCG ACCCTGCCGC TTACCGGATA CCTGTCCGCC |
| | 6061 TTTCTCCCTT CGGGAAGCGT GGCGCTTTCT CATAGCTCAC GCTGTAGGTA TCTCAGTTCG |
| | 6121 GTGTAGGTCG TTCGCTCCAA GCTGGGCTGT GTGCACGAAC CCCCCGTTCA GCCCGACCGC |
| | 6181 TGCGCCTTAT CCGGTAACTA TCGTCTTGAG TCCAACCCGG TAAGACACGA CTTATCGCCA |
| | 6241 CTGGCAGCAG CCACTGGTAA CAGGATTAGC AGAGCGAGGT ATGTAGGCGG TGCTACAGAG |
| | 6301 TTCTTGAAGT GGTGGCCTAA CTACGGCTAC ACTAGAAGAA CAGTATTTGG TATCTGCGCT |
| | 6362 CTGCTGAAGC CAGTTACCTT CGGAAAAAGA GTTGGTAGCT CTTGATCCGG CAAACAAACC |
| | 6421 ACCGCTGGTA GCGGTGGTTT TTTTGTTTGC AAGCAGCAGA TTACGCGCAG AAAAAAAGGA |
| | 6481 TCTCAAGAAG ATCCTTTGAT CTTTTCTACG GGGTCTGACG CTCAGTGGAA CGAAAACTCA |
| | 6541 CGTTAAGGGA TTTTGGTCAT GAGATTATCA AAAAGGATCT TCACCTAGAT CCTTTTAAAT |
| | 6601 TAAAAATGAA GTTTTAAATC AATCTAAAGT ATATATGAGT AAACTTGGTC TGACAGTTAC |
| | 6661 CAATGCTTAA TCAGTGAGGC ACCTATCTCA GCGATCTGTC TATTTCGTTC ATCCATAGTT |
| | 6721 GCCTGACTCC CCGTCGTGTA GATAACTACG ATACGGGAGG GCTTACCATC TGGCCCCAGT |
| | 6781 GCTGCAATGA TACCGCGAGA CCCACGCTCA CCGGCTCCAG ATTTATCAGC AATAAACCAG |
| | 6841 CCAGCCGGAA GGGCCGAGCG CAGAAGTGGT CCTGCAACTT TATCCGCCTC CATCCAGTCT |
| | 6901 ATTAATTGTT GCCGGGAAGC TAGAGTAAGT AGTTCGCCAG TTAATAGTTT GCGCAACGTT |
| | 6961 GTTGCCATTG CTACAGGCAT CGTGGTGTCA CGCTCGTCGT TTGGTATGGC TTCATTCAGC |
| | 7021 TCCGGTTCCC AACGATCAAG GCGAGTTACA TGATCCCCCA TGTTGTGCAA AAAAGCGGTT |
| | 7081 AGCTCCTTCG GTCCTCCGAT CGTTGTCAGA AGTAAGTTGG CCGCAGTGTT ATCACTCATG |
| | 7141 GTTATGGCAG CACTGCATAA TTCTCTTACT GTCATGCCAT CCGTAAGATG CTTTTCTGTG |
| | 7201 ACTGGTGAGT ACTCAACCAA GTCATTCTGA GAATAGTGTA TGCGGCGACC GAGTTGCTCT |
| | 7261 TGCCCGGCGT CAATACGGGA TAATACCGCG CCACATAGCA GAACTTTAAA AGTGCTCATC |
| | 7321 ATTGGAAAAC GTTCTTCGGG GCGAAAACTC TCAAGGATCT TACCGCTGTT GAGATCCAGT |
| | 7381 TCGATGTAAC CCACTCGTGC ACCCAACTGA TCTTCAGCAT CTTTTACTTT CACCAGCGTT |
| | 7441 TCTGGGTGAG CAAAAACAGG AAGGCAAAAT GCCGCAAAAA AGGGAATAAG GGCGACACGG |
| | 7501 AAATGTTGAA TACTCATACT CTTCCTTTTT CAATATTATT GAAGCATTTA TCAGGGTTAT |
| | 7561 TGTCTCATGA GCGGATACAT ATTTGAATGT ATTTAGAAAA ATAAACAAAT AGGGGTTCCG |
| | 7621 CGCACATTTC CCCGAAAAGT GCCACCTGAC |

//

### References

1. Fire, A. et al. Potent and specific genetic interference by double-stranded RNA in Caenorhabditis elegans. Nature 391, 806-11. (1998).
2. McManus, M. T. & Sharp, P. A. Gene Silencing in Mammals by siRNAs. Nature Genetics Reviews In Press (2002).
3. Reinhart, B. J. et al. The 21-nucleotide let-7 RNA regulates developmental timing in Caenorhabditis elegans. Nature 403, 901-6. (2000).
4. Olsen, P. H. & Ambros, V. The lin-4 regulatory RNA controls developmental timing in Caenorhabditis elegans by blocking LIN-14 protein synthesis after the initiation of translation. Dev Biol 216, 671-80. (1999).
5. Moss, E. G., Lee, R. C. & Ambros, V. The cold shock domain protein LIN-28 controls developmental timing in C. elegans and is regulated by the lin-4 RNA. Cell 88, 637-46. (1997).
6. Wightman, B., Ha, I. & Ruvkun, G. Posttranscriptional regulation of the heterochronic gene lin-14 by lin-4 mediates temporal pattern formation in C. elegans. Cell 75, 855-62. (1993).
7. Kennerdell, J. R. & Carthew, R. W. Use of dsRNA-mediated genetic interference to demonstrate that frizzled and frizzled 2 act in the wingless pathway. Cell 95, 1017-26. (1998).
8. Stark, G. R., Kerr, I. M., Williams, B. R., Silverman, R. H. & Schreiber, R. D. How cells respond to interferons. Annu Rev Biochem 67, 227-64 (1998).
9. Elbashir, S. M., Lendeckel, W. & Tuschl, T. RNA interference is mediated by 21- and 22-nucleotide RNAs. Genes Dev 15, 188-200. (2001).
10. Elbashir, S. M. et al. Duplexes of 21-nucleotide RNAs mediate RNA interference in cultured mammalian cells. Nature 411, 494-8. (2001).
11. McManus, M. T., Haines, B. B., Dillon, C.P., Whitehurst, C.E., van Parijs, L., Chen, J. & Sharp, P. A. siRNA-mediated gene silencing in T-cells. The Journal of Immunology, 2002, 169: 5754-5760.
12. Brummelkamp, T. R., Bernards, R. & Agami, R. A System for Stable Expression of Short Interfering RNAs in Mammalian Cells. Science 21, 21 (2002).
13. Paddison, P. J., Caudy, A. A., Bernstein, E., Hannon, G. J. & Conklin, D. S. Short hairpin RNAs (shRNAs) induce sequence-specific silencing in mammalian cells. Genes Dev 16, 948-58. (2002).
14. Sui, G. et al. A DNA vector-based RNAi technology to suppress gene expression in mammalian cells. Proc Natl Acad Sci USA 99, 5515-20. (2002).
15. Yu, J. Y., DeRuiter, S. L. & Turner, D. L. RNA interference by expression of short-interfering RNAs and hairpin RNAs in mammalian cells. Proc Natl Acad Sci U S A 23, 23 (2002).
16. Paul, C. P., Good, P. D., Winer, I. & Engelke, D. R. Effective expression of small interfering RNA in human cells. Nat Biotechnol 20, 505-8. (2002).
17. Bernstein, E., Caudy, A. A., Hammond, S. M. & Hannon, G. J. Role for a bidentate ribonuclease in the initiation step of RNA interference. Nature 409, 363-6. (2001).
18. Martinez, J., Patkaniowska, A., Urlaub, H., Luhrrmann, R. & Tuschl, T. Single-Stranded Antisense siRNAs Guide Target RNA Cleavage in RNAi. Cell 110, 563-574 (2002).
19. Brummelkamp, T. R., Bernards, R., and Agami, R. Stable suppression of tumorigenicity by virus-mediated RNA interference. Cancer Cell (2002).
20. Naldini, L. Lentiviruses as gene transfer agents for delivery to non-dividing cells. Curr Opin Biotechnol 9, 457-63 (1998).
21. Naldini, L. et al. In vivo gene delivery and stable transduction of nondividing cells by a lentiviral vector. Science 272, 263-7 (1996).
22. Jaenisch, R., Fan, H. & Croker, B. Infection of preimplantation mouse embryos and of newborn mice with leukemia virus: tissue distribution of viral DNA and RNA and leukemogenesis in the adult animal. Proc Natl Acad Sci USA 72, 4008-12 (1975).
23. Pfeifer, A., Ikawa, M., Dayn, Y. & Verma, I. M. Transgenesis by lentiviral vectors: lack of gene silencing in mammalian embryonic stem cells and preimplantation embryos. Proc Natl Acad Sci USA 99, 2140-5 (2002).
24. Lois, C., Hong, E. J., Pease, S., Brown, E. J. & Baltimore, D. Germline transmission and tissue-specific expression of transgenes delivered by lentiviral vectors. Science 295, 868-72 (2002).
25. Hacein-Bey-Abina, S. et al. Sustained correction of X-linked severe combined immunodeficiency by ex vivo gene therapy. N Engl J Med 346, 1185-93 (2002).
26. Tuschl, T. Expanding small RNA interference. Nat Biotechnol 20, 446-8 (2002).
27. Schmidt, E. V., Christoph, G., Zeller, R. & Leder, P. The cytomegalovirus enhancer: a pan-active control element in transgenic mice. Mol Cell Biol 10, 4406-11 (1990).
28. McManus, M. T., Petersen, C. P., Haines, B. B., Chen, J. & Sharp, P. A. Gene silencing using micro-RNA designed hairpins. Rna 8, 842-50. (2002).
29. Miyoshi, H., Blomer, U., Takahashi, M., Gage, F. H. & Verma, I. M. Development of a self-inactivating lentivirus vector. J Virol 72, 8150-7 (1998).
30. Devroe, E. a. S., PA. Retrovirus-delivered siRNA. BMC Biotechnology 2 (2002).
31. Willerford, D. M. et al. Interleukin-2 receptor alpha chain regulates the size and content of the peripheral lymphoid compartment. Immunity 3, 521-30 (1995).
32. Chen, J., Lansford, R., Stewart, V., Young, F. & Alt, F. W. RAG-2-deficient blastocyst complementation: an assay of gene function in lymphocyte development. Proc Natl Acad Sci USA 90, 4528-32 (1993).
33. Paddison, P. J. & Hannon, G. J. RNA interference: the new somatic cell genetics? Cancer Cell 2, 17-23. (2002).
34. Hannon, G. J. RNA interference. Nature 418, 244-51. (2002).
35. Mitchell, T. C. et al. Immunological adjuvants promote activated T cell survival via induction of Bc1-3. Nat Immunol 2, 397-402 (2001).
36. Eszterhas, S. K., Bouhassira, E. E., Martin, D. I. & Fiering, S. Transcriptional interference by independently regulated genes occurs in any relative arrangement of the genes and is influenced by chromosomal integration position. Mol Cell Biol 22, 469-79 (2002).
37. Fung-Leung, W. P. et al. CD8 is needed for development of cytotoxic T cells but not helper T cells. Cell 65, 443-9 (1991).
38. Hogquist, K. A. Signal strength in thymic selection and lineage commitment. Curr Opin Immunol 13,225-31 (2001).
39. Paddison, P. J., Caudy, A. A., Bernstein, E., Hannon, G. J. & Conklin, D. S. Short hairpin RNAs (shRNAs) induce sequence-specific silencing in mammalian cells. Genes Dev 16, 948-58 (2002).
40. Dull, T., Zufferey, R., Kelly, M., Mandel, R. J., Nguyen, M., Trono, D., & Naldini, L., A Third-Generation Lentivirus Vector with a Conditional Packaging System. Journal of Virology, 72(11), 8463-8471 (1998).
41. Zufferey, R., D. Nagy, R. J. Mandel, L. Naldini, and D. Trono. 1997. Multiply attenuated lentiviral vector achieves efficient gene delivery in vivo. Nat. Biotechnol. 15:871-875

### Equivalents

Those skilled in the art will recognize, or be able to ascertain using no more than routine experimentation, many equivalents to the specific embodiments of the invention described herein. The Examples below are provided to illustrate the invention and are not limiting. Alternative procedures known to one of ordinary skill in the art might also be used. The scope of the present invention is not intended to be limited to the above Description, but rather is as set forth in the appended claims.

1. A lentiviral vector comprising the following elements: a nucleic acid whose sequence includes (i) a functional packaging signal; (ii) a multiple cloning site (MCS); and (iii) at least one additional element selected from the group consisting of: a second MCS, a second MCS into which a heterologous nucleic acid is inserted, an HIV FLAP element, an expression-enhancing posttranscriptional regulatory element, a target site for a site-specific recombinase, and a self-inactivating (SIN) LTR, wherein the lentiviral vector is a lentiviral transfer plasmid or an infectious lentiviral particle.

2. The lentiviral vector of claim 1, wherein the vector comprises at least two elements selected from the group consisting of: a second MCS, a second MCS into which a heterologous nucleic acid is inserted, an HIV FLAP element, an expression-enhancing posttranscriptional regulatory element, a target site for a site-specific recombinase, and a self-inactivating (SIN) LTR.

3. The lentiviral vector of claim 1, wherein the vector comprises at least three elements selected from the group consisting of: a second MCS, a second MCS into which a heterologous nucleic acid is inserted, an HIV FLAP element, an expression-enhancing posttranscriptional regulatory element, a target site for a site-specific recombinase, and a self-inactivating (SIN) LTR.

4. The lentiviral vector of claim 1, wherein the vector comprises at least four elements selected from the group consisting of: a second MCS, a second MCS into which a heterologous nucleic acid is inserted, an HIV FLAP element, an expression-enhancing posttranscriptional regulatory element, a target site for a site-specific recombinase, and a self-inactivating (SIN) LTR.

5. The lentiviral vector of claim 1, wherein the vector comprises a second MCS, an HIV FLAP element, an expression-enhancing posttranscriptional regulatory element, a target site for a site-specific recombinase, and a self-inactivating (SIN) LTR.

6. The lentiviral vector of claim 1, wherein the vector comprises a second MCS into which a heterologous nucleic acid is inserted, an HIV FLAP element, an expression-enhancing posttranscriptional regulatory element, a target site for a site-specific recombinase, and a self-inactivating (SIN) LTR.

7. The lentiviral vector of claim 1, wherein the additional element is a second MCS.

8. The lentiviral vector of claim 1, wherein the additional element is a second MCS into which a heterologous nucleic acid is inserted.

9. The lentiviral vector of claim 1, wherein the vector has unique restriction sites for at least 4 enzymes selected from the group consisting of NotI, ApaI, XhoI, XbaI, HpaI, NheI, PacI, NsiI, SphI, Sma/Xma, AccI, BamHI, and SphI.

10. The lentiviral vector of claim 1, wherein the vector has unique restriction sites for at least 5, at least 6, at least 7, at least 8, at least 9, at least 10, at least 11, at least 12, or at least 13 enzymes selected from the group consisting of NotI, Apal, XhoI, XbaI, HpaI, NheI, PacI, NsiI, SphI, Sma/Xma, AccI, BamHI, and SphI.

11. The lentiviral vector of claim 1, wherein the additional element is an HIV FLAP element.

12. The lentiviral vector of claim 1, wherein the additional element is an expression-enhancing posttranscriptional regulatory element.

13. The lentiviral vector of claim 12, wherein the expression-enhancing posttranscriptional regulatory element is a WRE.

14. The lentiviral vector of claim 1, wherein the additional element is a target site for a site-specific recombinase.

15. The lentiviral vector of claim 14, wherein the site is a loxP site.

16. The lentiviral vector of claim 1, wherein the lentiviral vector is a lentiviral transfer plasmid.

17. The lentiviral transfer plasmid of claim 16, wherein the plasmid has a size of less than 10 kB.

18. The lentiviral transfer plasmid of claim 16, wherein the plamid has a size of less than 9 kB.

19. The lentiviral transfer plasmid of claim 16, wherein the plasmid has a size of less than 8 kB.

20. The lentiviral transfer plasmid of claim 16, wherein the plasmid has a size of less than 7 kB.

21. The lentiviral transfer plasmid of claim 16, wherein the plasmid has a size of approximately 6 kB.

22. The lentiviral vector of claim 1, wherein the lentiviral vector is an infectious lentiviral particle.

23. The lentiviral vector of claim 1, further comprising: a heterologous promoter or promoter-enhancer.

24. The lentiviral vector of claim 23, wherein the heterologous promoter or promoter-enhancer is selected from the group consisting of: the CMV promoter, the CMV promoter-enhancer, and the ubiquitin C promoter.

25. The lentiviral vector of claim 24, wherein the heterologous promoter is an inducible promoter.

26. The lentiviral vector of claim 24, wherein the heterologous promoter is a cell type specific or tissue specific promoter.

27. The lentiviral vector of claim 23, wherein the heterologous promoter is an RNA polymerase promoter.

28. The lentiviral vector of claim 27, wherein the RNA polymerase promoter is an RNA polymerase III promoter.

29. The lentiviral vector of claim 28, wherein the RNA polymerase III promoter is a U6 promoter.

30. The lentiviral vector of claim 28, wherein the RNA polymerase III promoter is an H1 promoter.

31. The lentiviral vector of claim 27, wherein the RNA polymerase promoter is an RNA polymerase II promoter.

32. The lentiviral vector of claim 23, further comprising a second heterologous promoter or promoter-enhancer.

33. The lentiviral vector of claim 1, further comprising a heterologous nucleic acid encoding a selectable marker operably linked to a promoter.

34. The lentiviral vector of claim 1, further comprising a heterologous nucleic acid encoding a reporter molecule operably linked to a promoter.

35. The lentiviral vector of claim 34, wherein the reporter molecule is selected from the group consisting of: GFP, EGFP, dsRed, dsRed2, cyan fluorescent protein, yellow fluorescent protein, blue fluorescent protein, dsRed, dsRed2, luciferase, and aequorin.

36. The lentiviral vector of claim 34, further comprising an RNA polymerase promoter.

37. The lentiviral vector of claim 36, wherein the RNA polymerase promoter is an RNA polymerase III promoter.

38. The lentiviral vector of claim 1, wherein the lentiviral vector is a transfer plasmid, further comprising a genetic element sufficient for stable maintenance of the transfer plasmid as an episome within mammalian cells.

39. A lentiviral vector comprising an RNA polymerase III promoter.

40. The lentiviral vector of claim 39, wherein the RNA polymerase III promoter is a U6 promoter.

41. The lentiviral vector of claim 39, wherein the RNA polymerase III promoter is an H1 promoter.

42. The lentiviral vector of claim 39, further comprising a heterologous nucleic acid encoding a reporter molecule.

43. A lentiviral vector having a sequence as set forth in SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO:9.

44. A collection of at least two of the lentiviral vectors of claim 43.

45. The collection of claim 44, wherein the collection includes a vector comprising a first heterologous promoter element and a vector comprising a second heterologous promoter element different from the first promoter element.

46. The collection of claim 44, wherein the collection includes a vector comprising a first heterologous reporter gene and a vector comprising a second reporter gene different from the first reporter gene.

47. A lentiviral vector having a sequence that differs by not more than 100 nucleotides from the sequence set forth in SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

48. A collection of at least two of the lentiviral vectors of claim 47.

49. The collection of claim 47, wherein the collection includes a vector comprising a first heterologous promoter element and a vector comprising a second heterologous promoter element different from the first promoter element.

50. The collection of claim 47, wherein the collection includes a vector comprising a first heterologous reporter gene and a vector comprising a second reporter gene different from the first reporter gene.

51. A lentiviral vector having a sequence that differs by not more than X nucleotides from the sequence set forth in SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9, where X represents any number between 1 and 99, inclusive.

52. A collection of at least two of the lentiviral vectors of claim 51.

53. A collection of at least two of the lentiviral vectors of claim 51.

54. The collection of claim 53, wherein the collection includes a vector comprising a first heterologous promoter element and a vector comprising a second heterologous promoter element different from the first promoter element.

55. The collection of claim 53, wherein the collection includes a vector comprising a first heterologous reporter gene and a vector comprising a second reporter gene different from the first reporter gene.

56. A three-plasmid lentiviral expression system comprising:
(a) a first plasmid whose sequence comprises a nucleic acid sequence of at least part of a lentiviral genome, wherein the plasmid (i) contains at least one defect in at least one gene encoding a lentiviral structural protein, and (ii) lacks a functional packaging signal;
(b) a second plasmid whose sequence comprises a nucleic acid sequence of a virus, wherein the plasmid (i) expresses a viral envelope protein, and (ii) lacks a functional packaging signal; and
(c) a third plasmid whose nucleic acid sequence includes (i) a functional packaging signal; (ii) a multiple cloning site (MCS); and (iii) at least one additional element selected from the group consisting of: a second MCS, a second MCS into which a heterologous nucleic acid is inserted; an HIV FLAP element, an expression-enhancing posttranscriptional regulatory element, a target site for a site-specific recombinase, and a self-inactivating (SIN) LTR.

57. A four plasmid lentiviral expression system comprising the three plasmid lentiviral expression system of claim 56, further comprising a fourth plasmid comprising a nucleic acid segment that encodes Rev, operably linked to a promoter.

58. A cell comprising the lentiviral vector of claim 1.

59. The cell of claim 58, wherein the cell comprises a nucleic acid or nucleic acids having sequences encoding Gag, Pol, and Env proteins.

60. A cell comprising a provirus derived from the lentiviral vector of claim 1.

61. A transgenic animal, at least some of whose cells contain the lentiviral vector of claim 1.

62. A transgenic animal, at least some of whose cells contain a provirus derived from the lentiviral vector of claim 1.

63. A method of creating a producer cell line comprising introducing the lentiviral vector of claim 1 into a host cell, wherein the lentiviral vector is a transfer plasmid; and introducing a packaging plasmid and an envelope plasmid into the host cell.

64. A method of producing lentiviral particles comprising
(i) introducing the lentiviral vector of claim I into a helper cell, wherein the lentiviral vector is a transfer plasmid comprising a genetic element sufficient for stable maintenance of the plasmid as an episome in mammalian cells, into a helper cell that expresses proteins required for production of infectious lentiviral particles; and
(ii) culturing the cell for a period sufficient to allow production of lentiviral particles.

65. A method of producing lentiviral particles comprising
(i) introducing the lentiviral vector of claim 1, which lentiviral vector is a lentiviral transfer plasmid comprising a genetic element sufficient for stable maintenance of the transfer plasmid as an episome in mammalian cells, into a helper cell that expresses a protein required for production of lentiviral particles, wherein expression of the protein is under control of an inducible promoter;
(ii) inducing expression of the protein required for production of lentiviral particles; and
(iii) culturing the cell for a period sufficient to allow production of lentiviral particles.

66. A method of expressing a heterologous nucleic acid in a target cell comprising
introducing a lentiviral vector of claim 1 into the target cell, wherein the lentiviral vector comprises a heterologous nucleic acid operably linked to a promoter; and
expressing the heterologous nucleic acid therein.

67. A method for achieving controlled expression of a heterologous nucleic acid in a cell comprising steps of:
(i) providing a modified lentiviral vector comprising a heterologous nucleic acid inserted between sites for a recombinase;
(ii) introducing the modified lentiviral vector or a portion thereof including at least the sites for the recombinase and the region between the sites into the cell and;
(iii) subsequently inducing expression of the recombinase within the cell, thereby preventing expression of the heterologous nucleic acid within the cell.

68. The method of claim 67, wherein the providing step comprises inserting the heterologous nucleic acid into a lentiviral vector between sites for a recombinase, thereby producing a modified lentiviral vector.

69. The method of claim 67, wherein the cell is a mammalian cell.

70. A method for expressing a transcript in a mammal in a cell type or tissue-specific manner comprising:
(i) delivering a lentiviral vector to cells of the mammal, wherein the lentiviral vector comprises a heterologous nucleic acid operably linked to a promoter so that transcription from the promoter results in synthesis of the transcript, and wherein the heterologous nucleic acid is located between sites for a site-specific recombinase; and
(ii) inducing expression of the site-specific recombinase in a subset of the cells of the mammal, thereby preventing synthesis of the transcript within those cells.

71. The method of claim 67 or claim 70, wherein the step of inducing the site-specific recombinase comprises introducing a vector encoding the site-specific recombinase into the cell.

72. The method of claim 67 or claim 70, wherein expression of the site-specific recombinase is under control of a cell type specific or tissue specific promoter.

73. The method of claim 67 or claim 70, wherein the sites are loxP sites and the site-specific recombinase is loxP.

74. A lentiviral vector whose presence within a cell results in transcription of one or more ribonucleic acids (RNAs) that self-hybridize or hybridize to each other to form a short hairpin RNA or short interfering RNA that inhibits expression of at least one target transcript in the cell.

75. The lentiviral vector of claim 74, wherein the vector provides a template for synthesis of an RNA that self-hybridizes to form an shRNA that is targeted to the transcript.

76. The lentiviral vector of claim 75, wherein the shRNA comprises a loop having a sequence set forth in SEQ ID NO: 10.

77. The lentiviral vector of claim 74, wherein the vector provides a template for synthesis of complementary RNAs that hybridize with each other to form an siRNA that is targeted to the transcript.

78. The lentiviral vector of claim 74, wherein the vector comprises a nucleic acid segment operably linked to a promoter, so that transcription from the promoter results in synthesis of one or more RNAs that self-hybridize or hybridize with each other to form an shRNA or siRNA targeted to the transcript.

79. The composition of claim 74, wherein the lentiviral vector is a lentiviral transfer plasmid.

80. The composition of claim 74, wherein the lentiviral vector is an infectious lentiviral particle.

81. The lentiviral vector of claim 74, wherein:
the shRNA or siRNA comprises a base-paired region approximately 19 nucleotides long.

82. The lentiviral vector of claim 74, wherein:
the shRNA or siRNA comprises a base-paired region and at least one single-stranded overhang.

83. The lentiviral vector of claim 74 wherein:
the siRNA or shRNA comprises a 3′ overhang consisting of at least two pyrimidines.

84. The lentiviral vector of claim 83, wherein the 3′ overhang is UU.

85. The lentiviral vector of claim 74, wherein:
the shRNA or siRNA comprises a region that is precisely complementary with a region of the target transcript.

86. The lentiviral vector of claim 74, wherein the siRNA or shRNA is present at a level sufficient to reduce the level of the target transcript or its encoded protein by at least about 2 fold.

87. The lentiviral vector of claim 74, wherein the siRNA or shRNA is present at a level sufficient to reduce the level of the target transcript or its encoded protein by at least about 5 fold.

88. The lentiviral vector of claim 74, wherein the siRNA or shRNA is present at a level sufficient to reduce the level of the target transcript or its encoded protein by at least about 10 fold.

89. The lentiviral vector of claim 74, wherein the siRNA or shRNA is present at a level sufficient to reduce the level of the target transcript or its encoded protein by at least about 25 fold.

90. The lentiviral vector of claim 74, wherein the lentiviral vector comprises:
(i) a functional packaging signal;
(ii) a multiple cloning site (MCS); and
(iii) at least one additional element selected from the group consisting of: a second MCS, a second MCS into which a heterologous promoter or promoter-enhancer is inserted, an HIV FLAP element, an expression-enhancing posttranscriptional regulatory element, a target site for a site-specific recombinase, and a self-inactivating (SIN) LTR.

91. A composition comprising:
the lentiviral vector of claim 74; and
a delivery agent that enhances delivery of the vector to cells.

92. A pharmaceutical composition comprising:
the lentiviral vector of claim 74; and
a pharmaceutically acceptable carrier.

93. A three plasmid lentiviral expression system comprising (i) a lentiviral transfer plasmid comprising a heterologous nucleic acid operably linked to a promoter, so that transcription of the heterologous nucleic acid produces one or more RNAs that self-hybridize or hybridize with each other to form an shRNA or siRNA targeted to a target transcript; (ii) a packaging plasmid; and (iii) an Env-coding plasmid.

94. A four plasmid lentiviral expression system comprising the three plasmid lentiviral expression system of claim 93, further comprising a fourth plasmid comprising a nucleic acid segment that encodes Rev, operably linked to a promoter.

95. A method of inhibiting or reducing the expression of a target transcript in a cell comprising delivering the lentiviral vector of claim 74 to the cell.

96. The method of claim 95, wherein the lentiviral vector comprises:
(i) a functional packaging signal;
(ii) a multiple cloning site (MCS); and
(iii) at least one additional element selected from the group consisting of: a second MCS, a second MCS into which a heterologous promoter or promoter-enhancer is inserted, an HIV FLAP element, an expression-enhancing posttranscriptional regulatory element, a target site for a site-specific recombinase, and a self-inactivating (SIN) LTR.

97. The method of claim 95, wherein the cell is a mammalian cell.

98. The method of claim 95, wherein the cell is a primary cell.

99. The method of claim 95, wherein the primary cell is a T cell.

100. The method of claim 95, wherein the cell is a non-dividing cell.

101. The method of claim 95, wherein the cell is an embryonic stem cell.

102. The method of claim 95, wherein the cell is a single-cell embryo.

103. The method of claim 95, wherein the lentiviral vector is a lentiviral transfer plasmid.

104. The method of claim 95, wherein the lentiviral vector is an infectious lentiviral particle.

105. The method of claim 95, wherein the ribonucleic acid comprises complementary regions that self-hybridize to form a short hairpin RNA targeted to the transcript.

106. A method of reversibly inhibiting or reducing expression of a target transcript in a cell comprising steps of:
(i) delivering a lentiviral vector to the cell, wherein presence of the lentiviral vector within the cell results in synthesis of one or more RNAs that self-hybridize or hybridize with each other to form an shRNA or siRNA that inhibits expression of the target transcript, wherein the lentiviral vector comprises a nucleic acid segment located between sites for a site-specific recombinase, which nucleic acid segment provides a template for transcription of the one or more RNAs; and (ii) inducing expression of the site-specific recombinase within the cell, thereby preventing synthesis of at least one of the RNAs.

107. The method of claim 105, wherein the cell is a mammalian cell.

108. The method of claim 105, wherein the recombinase is Cre and the sites are loxP sites.

109. The method of claim 105, wherein the lentiviral vector is a lentiviral transfer plasmid.

110. The method of claim 105, wherein the lentiviral vector is a lentiviral particle.

111. The method of claim 105, wherein the lentiviral vector provides a template for synthesis of an RNA comprising complementary portions that hybridize to form an shRNA.

112. A method for reversibly inhibiting or reducing expression of a transcript in a mammal in a cell type or tissue-specific manner comprising:
(i) delivering to the mammal a lentiviral vector whose presence within a cell results in synthesis of one or more RNAs that self-hybridize or hybridize with each other to form an shRNA or siRNA that inhibits expression of the target transcript, wherein the lentiviral vector comprises a nucleic acid segment located between sites for a site-specific recombinase, which nucleic acid segment provides a template for transcription of the RNA; and
(ii) inducing expression of the site-specific recombinase in a subset of the cells of the mammal, thereby preventing synthesis of at least one of the RNAs within the subset of cells.

113. The method of claim 112, wherein the recombinase is Cre and the sites are loxP sites.

114. The method of claim 112, wherein the lentiviral vector is a lentiviral transfer plasmid.

115. The method of claim 112, wherein the lentiviral vector is a lentiviral particle.

116. The method of claim 112, wherein the lentiviral vector provides a template for synthesis of an RNA comprising complementary portions that hybridize to form an shRNA.

117. A method of treating or preventing infection by an infectious agent, the method comprising steps of:
administering to a subject prior to, simultaneously with, or after exposure of the subject to the infectious agent, a composition comprising an effective amount of a lentiviral vector, wherein presence of the lentiviral vector in a cell results in synthesis of one or more RNAs that self-hybridize or hybridize with each other to form an shRNA or siRNA that is targeted to a transcript produced during infection by the infectious agent, which transcript is characterized in that reduction in levels of the transcript delays, prevents, or inhibits one or more aspects of infection by or replication of the infectious agent.

118. The method of claim 117, wherein the lentiviral vector provides a template for synthesis of an RNA that comprises complementary portions that hybridize to form an shRNA.

119. A method of treating or preventing a disease or clinical condition, the method comprising:
removing a population of cells from a subject at risk of or suffering from disease or clinical condition;
engineering or manipulating the cells to contain an effective amount of an siRNA or shRNA targeted to a transcript, which transcript is characterized in that its degradation delays, prevents, or inhibits one or more aspects of the disease or clinical condition; and
returning at least a portion of the cells to the subject.

120. The method of claim 119 wherein:
the engineering or manipulating step comprises introducing a lentiviral vector into the cells, wherein presence of the lentiviral vector in a cell results in synthesis of one or more RNAs that self-hybridize or hybridize with each other to form an shRNA or siRNA targeted to the transcript.

121. The method of claim 119, wherein:
the cells comprise stem cells.

122. The method of claim 121, wherein:
the stem cells are peripheral blood stem cells.

123. The method of claim 119, further comprising:
expanding at least a portion of the cells in culture.

124. A kit comprising (a) a lentiviral transfer plasmid comprising a nucleic acid sequence including (i) a functional packaging signal; (ii) a multiple cloning site (MCS) into which a heterologous gene may be inserted; and (iii) at least one additional element selected from the group consisting of: a second MCS, an HIV FLAP element, a heterologous promoter, a heterologous enhancer, an expression-enhancing posttranscriptional regulatory element, a target site for a site-specific recombinase, and a self-inactivating (SIN) LTR; and one or more of the following items: (b) a packaging mix comprising one or more plasmids that collectively provide nucleic acid sequences coding for retroviral or lentiviral Gag and Pol proteins and an envelope protein; (c) cells (e.g., a cell line) that are permissive for production of lentiviral particles such as 293T cells; (d) packaging cells, e.g., a cell line that is permissive for production of lentiviral particles and provides the proteins Gag, Pol, Env, and, optionally, Rev; (e) cells suitable for use in titering lentiviral particles; a transfection-enhancing agent such as Lipofectamine; (f) a selection agent such as an antibiotic, preferably corresponding to an antibiotic resistanc gene in the lentiviral transfer plasmid; (g) instructions for use; (h) a positive control plasmid.


SEQUENCE LISTING (39 sequences)

SEQ ID NO: 1 - DNA, 9710 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcaggaatctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatttgataccgcgggcccgggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaag

SEQ ID NO: 2 - DNA, 6027 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttaacgcgctagccgttaattaagcctcgaggtcgacggtatcgataagctcgcttcacgagattccagcaggtcgagggacctaataacttcgtatagcatacattatacgaagttatattaagggttccaagcttaagcggccgccgatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 3 - DNA, 6748 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttaacgcgctagccgttaattaagcctcgaggtcgacggtatcgataagctcgcttcacgagattccagcaggtcgagggacctaataacttcgtatagcatacattatacgaagttatattaagggttccaagcttaagcggccgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 4 - DNA, 6706 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttaacgcgctagccgttaattaagcctcgaggtcgacggtatcgataagctcgcttcacgagattccagcaggtcgagggacctaataacttcgtatagcatacattatacgaagttatattaagggttccaagcttaagcggccgcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 5 - DNA, 7248 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttgatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaacgcgctagccgttaattaagcctcgaggtcgacggtatcgataagctcgcttcacgagattccagcaggtcgagggacctaataacttcgtatagcatacattatacgaagttatattaagggttccaagcttaagcggccgccgatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 6 - DNA, 7969 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttgatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaacgcgctagccgttaattaagcctcgaggtcgacggtatcgataagctcgcttcacgagattccagcaggtcgagggacctaataacttcgtatagcatacattatacgaagttatattaagggttccaagcttaagcggccgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 7 - DNA, 7927 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttgatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctaacgcgctagccgttaattaagcctcgaggtcgacggtatcgataagctcgcttcacgagattccagcaggtcgagggacctaataacttcgtatagcatacattatacgaagttatattaagggttccaagcttaagcggccgcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgatgcatgccccgggatggcgcgccatggatccgcgaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 8 - DNA, 7350 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacggttaacgcgctagccgttaattaagcctcgaggtcgacggtatcgataagctcgcttcacgagattccagcaggtcgagggacctaataacttcgtatagcatacattatacgaagttatattaagggttccaagcttaagcggccgcgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 9 - DNA, 7650 bp, Artificial
  lentiviral vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgcggtgaccctcgaggtcgacggtatcgataagctcgcttcacgagattccagcaggtcgagggacctaataacttcgtatagcatacattatacgaagttatattaagggttccaagcttaagcggccgcgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 10 - DNA, 9 bp, Artificial
  Loop sequence
ttcaagaga

SEQ ID NO: 11 - DNA, 38 bp, Artificial
  Sense oligonucleotides
cgctctagacggttaacgcgctagccgttaattaagcc

SEQ ID NO: 12 - DNA, 46 bp, Artificial
  Antisense oligonucleotide
tcgaggcttaattaacggctagcgcgttaaccgtctagagcgggcc

SEQ ID NO: 13 - DNA, 43 bp, Artificial
  MCS sense
ggccgccgatgcatgccccgggatggcgcgccatggatccgcg

SEQ ID NO: 14 - DNA, 43 bp, Artificial
  MCS antisense
aattcgcggatccatggcgcgccatcccggggcatgcatcggc

SEQ ID NO: 15 - DNA, 31 bp, Artificial
  EGFP/5′ NotI oligonucleotides
cggcggccgcgccaccatggtgagcaagggc

SEQ ID NO: 16 - DNA, 30 bp, Artificial
  EGFP/3′NsiI oligonucleotides
cgatgcatcttgtacagctcgtccatgccg

SEQ ID NO: 17 - DNA, 31 bp, Artificial
  deRed2/5′ NotI oligonucleotides
cggcggccgcgccaccatggcctcctccgag

SEQ ID NO: 18 - DNA, 30 bp, Artificial
  dsRed2/3′ Nsil oligonucleotides
cgatgcatcaggaacaggtggtggcggccc

SEQ ID NO: 19 - DNA, 34 bp, Artificial
  5′CMV/NotI oligonucleotides
cggcggccgcgtggataaccgtattaccgccatg

SEQ ID NO: 20 - DNA, 35 bp, Artificial
  3′ EGFP/stop/EcoRI oligonucleotides
cggaattcctacttgtacagctcgtccatgccgag

SEQ ID NO: 21 - DNA, 30 bp, Artificial
  5′ Xbal/U6 oligonucleotides
gctctagagatccgacgccgccatctctag

SEQ ID NO: 22 - DNA, 43 bp, Artificial
  3′ Xhol/BstEII/Hpal/U6 oligonucleotides
gcctcgagggtcaccgcgcgttaacaaggcttttctccaaggg

SEQ ID NO: 23 - DNA, 74 bp, Artificial
  5′ EcoRI/LoxFix/Pcil oligonucleotides
gcgaattcgtcgagggacctaataacttcgtatagcatacattatacgaagttatacatgtttaagggttccgg

SEQ ID NO: 24 - DNA, 25 bp, Artificial
  3′ Pf1m1/Rev oligonucleotide
aaggagctgacaggtggtggcaatg

SEQ ID NO: 25 - DNA, 20 bp, Artificial
  CD8 shRNA duplex sequence
tgctacaactactacatgac

SEQ ID NO: 26 - DNA, 59 bp, Artificial
  Mena + sense
tgtcctgtgcctggcctactttcaagagaagtaggccaggcacaggactttttggaaac

SEQ ID NO: 27 - DNA, 63 bp, Artificial
  Mena + antisense
tcgagttcccaaaaagtcctgtgcctggcctacttctcttgaaagtaggccaggcacaggaca

SEQ ID NO: 28 - DNA, 58 bp, Artificial
  Beta-catenin sense
tgtccagcgcttggctgaacttcaagagtgttcagccaagcgctggactttttggaaa

SEQ ID NO: 29 - DNA, 62 bp, Artificial
  Beta antisense
tcgatttccaaaaagtccagcgcttggctgaacactcttgaagttcagccaagcgctggaca

SEQ ID NO: 30 - DNA, 72 bp, Artificial
  P53 sense
tggtctaagtggagcccttcgagtgttagaagcttgtgacactcggagggcttcacttgggctttttggaaa

SEQ ID NO: 31 - DNA, 77 bp, Artificial
  P53 antisense
tcgatttccaaaaaggcccaagtgaagccctccgagtgtcacaagcttctaacactcgaagggctccacttagacca

SEQ ID NO: 32 - DNA, 21 bp, Artificial
  Primer
tggtgggtacctagtggaacc

SEQ ID NO: 33 - DNA, 47 bp, Artificial
  Primer
aagcttaagcggccgcagaattcgtcgagggacctaataacgtatag

SEQ ID NO: 34 - DNA, 44 bp, Artificial
  Primer
gaattctgcggccgcttaagcttggaacccttaatataacttcg

SEQ ID NO: 35 - DNA, 21 bp, Artificial
  Primer
cgcttcacgagattccagcag

SEQ ID NO: 36 - DNA, 55 bp, Artificial
  CD8 sense
tgctacaactactacatgacttcaagagagtcatgtagtagttgtagcttttttg

SEQ ID NO: 37 - DNA, 59 bp, Artificial
  CD8 antisense
gttacaaaaaagctacaactactacatgactctcttgaagtcatgtagtagttgtagca

SEQ ID NO: 38 - DNA, 55 bp, Artificial
  CD25 sense
tgcattcacctaatcggctgttcaagagacagccgattaggtgaatgcttttttg

SEQ ID NO: 39 - DNA, 60 bp, Artificial
  CD25 antisense
gtcaccaaaaaagcattcacctaatcggctgtctcttgaacagccgattaggtgaatgca